(19) United States
(12) Patent Application Publication
(10) Pub. No.: US 2020/0140821 A1
(43) Pub. Date: May 7, 2020

(54) EX VIVO MEAT PRODUCTION

(71) Applicant: WILD TYPE, INC., San Francisco, CA (US)

(72) Inventors: Arye ELFENBEIN, San Francisco, CA (US); Justin Lee KOLBECK, Seattle, WA (US)

(21) Appl. No.: 16/620,326

(22) PCT Filed: Jun. 7, 2018
(86) PCT No.: PCT/US2018/036552

§ 371 Date: Dec. 6, 2019

Related U.S. Application Data
(60) Provisional application No. 62/516,575, filed on Jun. 7, 2017.
(60) Provisional application No. 62/653,332, filed on Apr. 5, 2018.

(51) Int. Cl. C12N 5/077; A23L 13/00; A23L 17/00; C12N 5/00

(52) U.S. Cl. CPC C12N 5/0658; A23L 13/00; A23L 17/00; C12N 2500/76; C12N 2500/34; C12N 2500/90; C12N 2500/74; C12N 5/0037

(57) ABSTRACT

Systems and methods for producing cell cultured food products. The cultured food products include sushi-grade fish meat, fish surimi, foie gras, and other food types. Various cell types are utilized to produce the food products and can include muscle, fat, and/or liver cells. The cultured food products are grown in pathogen-free culture conditions without exposure to toxins and other undesirable chemicals.

### CROSS-REFERENCE

This application claims the benefit of U.S. Provisional Application No. 62/516,575, filed Jun. 7, 2017, and U.S. Provisional Application No. 62/653,332, filed Apr. 5, 2018, both of which are incorporated herein by reference in their entirety.

### BACKGROUND OF THE INVENTION

Traditional meat production is a resource-intensive process that generates a significant environmental footprint. Domesticated animals are raised in agricultural settings requiring substantial quantities of fresh water, feed, land, and other resources. Similarly, consumption of fish is vulnerable to a number of problems including overfishing and by-catch as well as pollution caused by fisheries.

### SUMMARY OF THE INVENTION

Disclosed herein are methods of producing cultured fish meat for human consumption. Some such methods comprise: a) obtaining a population of self-renewing cells derived from fish; b) culturing the population of self-renewing cells in culture media comprising micro-scaffolds; c) inducing differentiation in the population of cells to form at least one of myocytes and adipocytes; and d) processing the population of cells into fish meat for human consumption. Various aspects incorporate at least one of the following elements. The fish meat is often sushi. In some instances, the fish meat is surimi. Sometimes, the fish meat is suitable for raw consumption. In certain cases, the fish meat is cooked. The fish meat is usually salmon meat. In certain aspects, the fish meat is sushi-grade salmon meat. Alternatively, the fish meat is sometimes tuna meat. Sometimes, the fish meat is sushi-grade tuna meat. In some cases, inducing differentiation in (c) causes the population of cells to form myocytes and adipocytes. In some cases, differentiation comprises transdifferentiation of cells into a different cell type. Oftentimes, the fish meat is composed of at least 50% high glycolytic and anaerobic muscle fibers. The population of cells is frequently derived from sea bass, tuna, mackerel, blue marlin, swordfish, yellowtail, salmon, or trout. Processing in (d) usually comprises combining the population of cells with a second population of cells composed of myocytes or adipocytes. In various aspects, the population of cells is isolated as embryonic stem cells. Sometimes, the population of cells has been modified to induce pluripotency. The population of cells is isolated as multipotent adult stem cells, in certain embodiments. Culturing typically comprises growing and expanding the population of cells in cell culture. Inducing differentiation often comprises exposing the population of cells to culture conditions that stimulate differentiation. Sometimes, inducing differentiation comprises exposing the population of cells to at least one growth factor that stimulates differentiation. In certain instances, culturing comprises growing the population of cells on a two dimensional surface. Alternatively, culturing comprises growing the population of cells on a three-dimensional scaffold. Culturing often comprises growing the population of cells on micro-scaffolds within a bioreactor, wherein the micro-scaffolds enable cell adhesion. Sometimes, the population of cells forms non-textured tissue after differentiation. In various aspects, culturing comprises growing the population of cells in a media formulation comprising at least one nutritional supplement. The at least one nutritional supplement usually comprises an omega-3 fatty acid. The at least one nutritional supplement comprises a polyunsaturated fatty acid, sometimes. In certain instances, the at least one nutritional supplement comprises a monounsaturated fatty acid. Sometimes, the population of cells is cultured using a non-serum media formulation. In many cases, the population of cells is cultured using a mushroom-based media formulation.

In some aspects, disclosed herein are methods for producing cultured fish tissue, the methods comprising: a) culturing a population of fish pre-adipocytes and a population of fish satellite cells; b) inducing differentiation in the population of fish pre-adipocytes to form adipocytes; c) inducing differentiation in the population of fish satellite cells to produce myocytes; d) co-culturing the adipocytes and myocytes; and e) processing the adipocytes and myocytes into fish tissue for human consumption. Various aspects include at least one of the following elements. Sometimes, the fish tissue comprises fast twitch muscle fibers. Oftentimes, the fish tissue is salmon tissue. In certain cases, the fish tissue is tuna tissue. The fish tissue is occasionally trout tissue. In many instances, the fish tissue is surimi. The fish tissue is often sushi. The fish tissue is made for raw human consumption, in some cases. The fish tissue is sometimes cooked for human consumption. In various aspects, the adipocytes and myocytes are co-cultured in a media formulation comprising at least one nutritional supplement. The at least one nutritional supplement usually comprises an omega-3 fatty acid. Sometimes, the at least one nutritional supplement comprises a polyunsaturated fatty acid. The at least one nutritional supplement comprises a monounsaturated fatty acid, on occasion. Oftentimes, a non-serum media formulation is used for cell culturing. In certain cases, a mushroom-based media formulation is used for cell culturing.

In some aspects, disclosed herein are methods for producing cultured fish tissue, the methods comprising: a) culturing a population of fish pre-adipocytes and a population of fish satellite cells, said populations adapted for suspension culture; b) inducing differentiation in the population of fish pre-adipocytes to form adipocytes; c) inducing differentiation in the population of fish satellite cells to form myocytes; d) co-culturing the adipocytes and myocytes; and e) processing the adipocytes and myocytes into fish tissue for human consumption.

In some aspects, disclosed herein are edible compositions comprising fish tissue produced from co-cultured myocytes and adipocytes.

In some aspects, disclosed herein are edible compositions comprising fish tissue produced from pre-adipocytes and satellite cells.

In some aspects, disclosed herein are methods of producing cultured fish meat for human consumption, the methods comprising: a) obtaining a population of pre-adipocytes and a population of satellite cells; b) adapting the population of pre-adipocytes and the population of satellite cells to suspension culture; c) inducing differentiation in the population of pre-adipocytes and the population of satellite cells; d) co-culturing the populations in suspension culture; and e) processing the populations into fish meat for human consumption. In some cases, differentiation comprises transdifferentiation of cells into a different cell type. Various aspects include at least one of the following elements. Sometimes, the fish meat is sushi. The fish meat is often surimi. In certain instances, the fish meat is suitable for raw consumption. Oftentimes, the fish meat is cooked. In various aspects, the fish meat is salmon meat. The fish meat is sushi-grade salmon meat, in certain cases. The fish meat is often tuna meat. Sometimes, the fish meat is sushi-grade tuna meat. Occasionally, the fish meat is trout meat. In many instances, the fish meat is composed of at least 50% high glycolytic and anaerobic muscle fibers. The population of pre-adipocytes is usually derived from sea bass, tuna, mackerel, blue marlin, swordfish, yellowtail, salmon, or trout. The population of satellite cells is often derived from sea bass, tuna, mackerel, blue marlin, swordfish, yellowtail, salmon, or trout. Co-culturing typically comprises growing and expanding the populations in cell culture. In certain cases, inducing differentiation comprises exposing the population of pre-adipocytes to at least one growth factor that stimulates differentiation into adipocytes. Sometimes, inducing differentiation comprises exposing the population of satellite cells to at least one growth factor that stimulates differentiation into myocytes. Culturing often comprises growing the population of cells within a bioreactor. In many cases, the myocytes and adipocytes form non-textured tissue after differentiation. The myocytes and adipocytes are often cultured in a media formulation comprising at least one nutritional supplement. The at least one nutritional supplement usually comprises an omega-3 fatty acid. In many instances, the at least one nutritional supplement comprises a polyunsaturated fatty acid. Occasionally, the at least one nutritional supplement comprises a monounsaturated fatty acid. Oftentimes, a non-serum media formulation is used for cell culturing. In certain cases, a mushroom-based media formulation is used for cell culturing.

In some aspects, disclosed herein are fish products suitable for human consumption comprising fish surimi produced from cultured myocytes and adipocytes.

In some aspects, disclosed herein are synthetic food products suitable for human consumption comprising fish meat derived from cultured satellite cells and pre-adipocytes.

In some aspects, disclosed herein are fish products suitable for human consumption comprising fish meat produced from myocytes and adipocytes grown in suspension culture.

In some aspects, disclosed herein are methods of producing cultured fish meat for human consumption, the methods comprising: a) obtaining a population fish pre-adipocytes capable of growing in suspension culture; b) obtaining a population of fish satellite cells capable of growing in suspension culture; c) inducing differentiation in the population of fish pre-adipocytes and the population of fish satellite cells to form adipocytes and myocytes; d) co-culturing the adipocytes and myocytes in suspension culture comprising at least one nutritional supplement; and e) processing the population of cells into fish meat for human consumption. Various aspects include at least one of the following elements. Sometimes, the fish meat is sushi. Oftentimes, the fish meat is surimi. In many cases, the fish meat is suitable for raw consumption. The fish meat is occasionally cooked. The fish meat is sometimes salmon meat. In certain instances, the fish meat is sushi-grade salmon meat. Sometimes, the fish meat is tuna meat. The fish meat is often sushi-grade tuna meat. The fish meat is composed of at least 50% high glycolytic and anaerobic muscle fibers, in various aspects. Typically, the population of cells is derived from sea bass, tuna, mackerel, blue marlin, swordfish, yellowtail, salmon, or trout. Oftentimes, inducing differentiation in (c) comprises exposing the population of pre-adipocytes and the population of satellite cells to culture conditions that stimulate differentiation. inducing differentiation in (c) usually comprises exposing the population of pre-adipocytes to at least one growth factor that stimulates differentiation. In certain instances, inducing differentiation in (c) comprises exposing the population of satellite cells to at least one growth factor that stimulate differentiation. The adipocytes and myocytes usually form non-textured tissue. Sometimes, the at least one nutritional supplement comprises an omega-3 fatty acid. In many cases, the at least one nutritional supplement comprises a polyunsaturated fatty acid. Sometimes, the at least one nutritional supplement comprises a monounsaturated fatty acid. Oftentimes, a non-serum media formulation is used for cell culturing. In certain cases, a mushroom-based media formulation is used for cell culturing. In some cases, the population of cells are transdifferentiated into at least one cell type. In some cases, the population of cells are transdifferentiated into at least one of hepatocytes, myocytes, and adipocytes.

In some aspects, disclosed herein are methods of producing cultured tissue for human consumption, the method comprising: a) obtaining a population of self-renewing cells; b) culturing the population of self-renewing cells; c) inducing differentiation in the population of self-renewing cells to form cultured tissue; and d) processing the cultured tissue for human consumption. Various aspects include at least one of the following elements. In some cases, obtaining the population of self-renewing cells comprises transitioning a population of cells from 2-dimensional adherent culture into 3-dimensional culture in a bioreactor. Sometimes, the population of self-renewing cells comprises differentiated cells that have become immortalized. Inducing differentiation in the population of self-renewing cells often comprises inducing transdifferentiation of cells in the population into myocytes, adipocytes, or a combination thereof. In some cases, the population of cells are transdifferentiated into at least one cell type. In some cases, the population of cells are transdifferentiated into at least one of hepatocytes, myocytes, and adipocytes. In certain instances, culturing comprises seeding the population of self-renewing cells on 3-dimensional micro-scaffolds. In some cases, the 3-dimensional micro-scaffolds promote cell growth, adhesion, differentiation, or a combination thereof. The 3-dimensional micro-scaffolds are conjugated to at least one factor promoting cell growth, adhesion, differentiation, or a combination thereof, in various aspects. Sometimes, the micro-scaffolds comprise at least one of hydrogel, chitosan, polyethylene terephthalate, collagen, elastin, heparan sulfate, chondroitin sulfate, keratan sulfate, hyaluronic acid, laminin, fibronectin, cellulose, hemicellulose, pectin, lignin, alginate, glucomannan, polycaprolactone (PCL), textured vegetable protein (TVP), textured soy protein (TSP), and acrylates. In certain instances, the population of self-renewing cells comprises at least one cell that has been modified to undergo inducible differentiation. In some instances, the at least one cell has been modified to incorporate: a) a first genetic construct comprising an open reading frame (ORF) of at least one pluripotency gene; and b) a second genetic construct comprising an open reading frame (ORF) of a regulatory factor configured to inactivate the at least one pluripotency gene. Often, the population of self-renewing cells comprises at least one cell that undergoes at least 50 cell divisions during culturing. In some cases, the regulatory factor is a recombinase, and the open reading frame (ORF) of at least one pluripotency gene is flanked by recombination sequences recognized by the recombinase such that expression of the recombinase catalyzes excision of the open reading frame (ORF) of at least one pluripotency gene. The second genetic construct comprises an ORF of at least one hepatocyte differentiation factor selected from Hepatocyte Nuclear Factor 1 Alpha (HNF1A), Forkhead Box A2 (FOXA2), and Hepatocyte Nuclear Factor 4 Alpha (HNF4A), in some instances. In various aspects, the second genetic construct comprises at least one myogenic factor selected from Myogenin (MyoG), Myogenic Differentiation 1 (MyoD), Myogenic Factor 6 (MRF4), and Myogenic Factor 5 (MYF5). The second genetic construct often comprises at least one adipogenic factor selected from Fatty Acid Binding Protein 4 (FABP4), Insulin-Responsive Glucose Transporter Type 4 (GLUT4), Adiponectin, C1Q And Collagen Domain Containing (ADIPOQ), 1-Acylglycerol-3-Phosphate O-Acyltransferase 2 (AGPAT2), Perilipin 1 (PLIN1), Leptin (LEP), and Lipoprotein Lipase (LPL). Sometimes, the second genetic construct further comprises: a) an open reading frame (ORF) of at least one differentiation gene; and b) an inducible promoter controlling expression of: i) the open reading frame (ORF) of the at least one differentiation gene; and ii) the open reading frame (ORF) of the regulatory factor. In certain cases, inducing differentiation comprises exposing the at least one cell to an induction agent to induce expression of the ORF of at least one cell lineage gene and the ORF of the regulatory factor. The method typically comprises removing the induction agent after the population of self-renewing cells has been treated with the induction agent and before being processed for human consumption in step d). Inducing differentiation comprises generating myotubes within the population of self-renewing cells, in certain cases. In many instances, inducing differentiation further comprises generating adipocytes within the population of self-renewing cells. Sometimes, the population of self-renewing cells comprises multipotent cells that are induced to differentiate into myocytes and adipocytes during step c). The multipotent cells often comprise a first subpopulation of myosatellite cells and a second subpopulation of pre-adipocytes. In some instances, inducing differentiation comprises generating hepatocytes within the population of self-renewing cells. The population of self-renewing cells is derived from an avian species selected from duck, goose, chicken, and turkey, in some aspects. The method often further comprises inducing steatosis within at least one of the hepatocytes. In certain instances, the population of self-renewing cells comprises at least one cell modified to express at least one gene for enhancing steatosis upon treatment with an induction agent. Sometimes, the at least one cell is stably transformed using a construct comprising an open reading frame (ORF) encoding ATF4, ZFP423, LPIN1, PPAR, APOC3, APOE, ORL1, PEMT, MTTP, SREBP, STAT3, or KLF6. 433. In various aspects, inducing steatosis comprises incubating the hepatocytes in a culture medium comprising at least nutritional supplement. The at least one nutritional supplement often comprises a polyunsaturated fatty acid, a monounsaturated fatty acid, or a combination thereof. In some cases, the at least one nutritional supplement comprises palmitic acid, oleic acid, docosahexaenoic acid, stearic acid, linoleic acid, linolenic acid, arachidonic acid, eicosapentaenoic acid, or a combination thereof. The cultured tissue comprises octopus, squid, or cuttlefish muscle cells, in certain aspects. Sometimes, the cultured tissue comprises fish muscle tissue. The population of self-renewing cells can be derived from sea bass, tuna, mackerel, blue marlin, swordfish, yellowtail, salmon, or trout. In many cases, the fish muscle tissue is combined with separately cultured fish fat tissue during step d). In some instances, the population of cells is cultured using a non-serum media formulation. The non-serum media formulation comprises a mushroom extract or soybean hydrolysate, in some embodiments.

Disclosed herein are methods of producing cultured meat for human consumption. Some such methods comprise: a) obtaining a population of self-renewing cells, said cells capable of growing in suspension culture; b) culturing the population of self-renewing cells in suspension; c) inducing differentiation in the population of cells to form at least one of myocytes and adipocytes; and d) processing the population of cells into meat for human consumption. Various aspects incorporate at least one of the following elements. Sometimes, the meat is fish meat. The fish meat is usually sushi. In some embodiments, the fish meat is surimi. Oftentimes, the fish meat is suitable for raw consumption. In certain cases, the fish meat is cooked. In certain cases, the fish meat is salmon meat. In certain aspects, the fish meat is sushi-grade salmon meat. In some cases, the fish meat is tuna meat. Sometimes, the fish meat is sushi-grade tuna meat. Oftentimes, inducing differentiation in c) causes the population of cells to form myocytes and adipocytes. The fish meat is usually composed of at least 50% high glycolytic and anaerobic muscle fibers. The population of cells is usually derived from sea bass, tuna, mackerel, blue marlin, swordfish, yellowtail, salmon, or trout. Processing in d) often comprises combining the population of cells with a second population of cells composed of myocytes or adipocytes. In certain cases, the population of cells is isolated as embryonic stem cells. Sometimes, the population of cells has been modified to induce pluripotency. Certain populations of cells are isolated as multipotent adult stem cells. Sometimes, the population of self-renewing cells are immortalized cells. Culturing typically comprises growing and expanding the population of cells in cell culture. Oftentimes, inducing differentiation comprises exposing the population of cells to culture conditions that stimulate differentiation. In some cases, differentiation comprises transdifferentiation of cells into a different cell type. Inducing differentiation comprises exposing the population of cells to at least one growth factor that stimulates differentiation, in some instances. Culturing sometimes comprises growing the population of cells on a two dimensional surface. Certain populations of cells form non-textured tissue after differentiation. In certain aspects, culturing comprises growing the population of cells in a media formulation comprising at least one nutritional supplement. Sometimes, the at least one nutritional supplement comprises an omega-3 fatty acid. In other cases, the at least one nutritional supplement comprises a polyunsaturated fatty acid. Occasionally, the at least one nutritional supplement comprises a monounsaturated fatty acid. Sometimes, the population of cells is cultured using a non-serum media formulation. In many cases, the population of cells is cultured using a mushroom-based media formulation.

Disclosed herein are methods of producing cultured cells having high lipid accumulation for human consumption. Some methods comprise: a) culturing a population of cells; b) inducing differentiation within the population of cells; c) inducing high lipid accumulation within the population of cells; and d) processing the population of cells for human consumption. Various aspects incorporate at least one of the following elements. In certain cases, the population of cells following differentiation comprises hepatocytes. Oftentimes, processing comprises using the population of cells as an ingredient in foie gras. The population of cells is derived from duck or goose, in some cases. The population of cells is sometimes derived from at least one of poultry and livestock. In certain instances, inducing high lipid accumulation comprises inducing steatosis. In some embodiments, high lipid accumulation is characterized by excess accumulation of cytoplasmic lipid droplets. Inducing high lipid accumulation often comprises exposing the population of cells to an exogenous compound that modulates at least one lipid metabolic pathway. In certain cases, inducing high lipid accumulation comprises exposing the population of cells to at least one of a toxin and a high lipid concentration. Sometimes, inducing high lipid accumulation comprises modulating at least one lipid metabolic pathway to enhance lipid retention within the population of cells. In some instances, inducing high lipid accumulation comprises altering at least one gene in within the population of cells to modulate lipid metabolism. Oftentimes, the population of cells following differentiation comprises liver, heart, kidney, stomach, intestine, lung, diaphragm, esophagus, thymus, pancreas, or tongue cells. Processing the population of cells for human consumption comprises blending the population of cells with cells having low lipid accumulation, in various aspects. The population of cells is sometimes isolated as embryonic stem cells. In certain cases, the population of cells has been modified to induce pluripotency. In some instances, the population of cells is isolated as multipotent adult stem cells. Culturing typically comprises growing and expanding the population of cells in cell culture. In certain aspects, inducing differentiation comprises exposing the population of cells to culture conditions that stimulate differentiation. In some embodiments, inducing differentiation comprises exposing the population of cells to at least one growth factor that stimulates differentiation. In some cases, differentiation comprises transdifferentiation of cells into a different cell type. Culturing sometimes comprises growing the population of cells on a two dimensional surface. In many cases, culturing comprises growing the population of cells on a three-dimensional scaffold. In certain instances, culturing comprises growing the population of cells on micro-scaffolds within a bioreactor, wherein the micro-scaffolds enable cell adhesion. In some embodiments, the population of cells does not require an adherence substrate for survival and proliferation. Sometimes, the population of cells is adapted to suspension culture. The population of cells often forms non-textured tissue after differentiation. The population of cells forms non-muscle tissue after differentiation, in some aspects. In various cases, culturing comprises growing the population of cells in a media formulation comprising at least one nutritional supplement. In some aspects, the at least one nutritional supplement comprises an omega-3 fatty acid. The at least one nutritional supplement frequently comprises at least one of a polyunsaturated fatty acid. Sometimes, the population of cells is cultured using a non-serum media formulation. In many cases, the population of cells is cultured using a mushroom-based media formulation.

Disclosed herein are methods of producing cultured non-textured tissue having high lipid content. Some such methods comprise: a) obtaining a population of differentiated cells capable of self-renewal; b) culturing the population of differentiated cells; c) manipulating at least one lipid metabolic pathway to induce steatosis in the population of differentiated cells such that the cells accumulate high lipid content; and d) processing the population of differentiated cells into non-textured tissue. Various aspects incorporate at least one of the following elements. In some cases, obtaining the population of differentiated cells capable of self-renewal comprises transforming differentiated cells into immortalized cells. Oftentimes, obtaining the population of differentiated cells capable of self-renewal comprises culturing differentiated cells until spontaneous mutations give rise to immortalized cells. In some cases, differentiation comprises transdifferentiation of cells into a different cell type. Sometimes, the population of differentiated cells comprises fibroblasts. In some instances, the population of differentiated cells are transdifferentiated into myocytes, adipocytes, or a combination thereof. The population of differentiated cells are derived from fish such as salmon or trout, in some aspects. The population of differentiated cells comprises hepatocytes, in certain instances. In many cases, processing comprises using the population of differentiated cells as an ingredient in foie gras. In certain embodiments, the population of differentiated cells is derived from duck or goose. The population of differentiated cells is oftentimes derived from at least one of poultry and livestock. Typically, steatosis is characterized by excess accumulation of cytoplasmic lipid droplets. In certain embodiments, manipulating the at least one lipid metabolic pathway comprises exposing the population of cells to an exogenous compound. Manipulating the at least one lipid metabolic pathway comprises exposing the population of differentiated cells to at least one of a toxin and a high lipid concentration, in some aspects. Alternatively, or in combination, manipulating the at least one lipid metabolic pathway comprises altering at least one gene in within the population of cells to modulate lipid metabolism. In many cases, the population of differentiated cells comprises liver, heart, kidney, stomach, intestine, lung, diaphragm, esophagus, thymus, pancreas, or tongue cells. Sometimes, processing the population of differentiated cells comprises blending the population of cells with cells having low lipid accumulation. In many aspects, culturing comprises growing and expanding the population of cells in cell culture. Culturing sometimes comprises growing the population of cells on a two dimensional surface. In certain aspects, culturing comprises growing the population of cells on a three-dimensional scaffold. In some cases, culturing comprises growing the population of cells on micro-scaffolds within a bioreactor, wherein the micro-scaffolds enable cell adhesion. The population of cells does not require an adherence substrate for survival and proliferation, in certain instances. Sometimes, the population of cells is adapted to suspension culture. Oftentimes, the population of differentiated cells forms non-textured tissue. In some cases, the population of cells forms non-muscle tissue. Culturing comprises growing the population of cells in a media formulation comprising at least one nutritional supplement, in many aspects. In certain instances, the at least one nutritional supplement comprises an omega-3 fatty acid. Sometimes, the at least one nutritional supplement comprises a polyunsaturated fatty acid. Sometimes, the at least one nutritional supplement comprises a monounsaturated fatty acid. Sometimes, the population of cells is cultured using a non-serum media formulation. In many cases, the population of cells is cultured using a mushroom-based media formulation.

Disclosed herein are methods of producing cultured non-muscle tissue for human consumption. Some such methods comprise: a) obtaining a population of self-renewing cells; b) culturing the population of self-renewing cells; c) inducing differentiation in the population of cells to form non-muscle tissue; and d) processing the cultured non-muscle tissue for human consumption. Various aspects incorporate at least one of the following elements. In some cases, differentiation comprises transdifferentiation of cells into a different cell type.

In some aspects, disclosed herein are methods for producing cultured tissue for human consumption, the methods comprising: obtaining a population of self-renewing cells; adapting the population of self-renewing cells to suspension culture; culturing the population of self-renewing cells; inducing differentiation in the population of cells to form cultured tissue; and processing the cultured tissue for human consumption. In some cases, differentiation comprises transdifferentiation of cells into a different cell type.

Disclosed herein are methods of producing cultured non-textured muscle tissue for human consumption. Some such methods comprise: a) obtaining a population of self-renewing cells; b) culturing the population of self-renewing cells; c) inducing differentiation in the population of cells to form non-textured muscle tissue; and d) processing the cultured non-textured muscle tissue for human consumption. Various aspects incorporate at least one of the following elements. In some cases, the non-textured muscle tissue is octopus, squid, or cuttlefish muscle. Sometimes, the non-textured muscle tissue is fish muscle tissue. In certain instances, the fish muscle tissue comprises high glycolytic and anaerobic muscle fibers. The high glycolytic and anaerobic muscle fibers often make up at least 80% of the fish muscle tissue. In some cases, the population of cells is derived from sea bass, tuna, mackerel, blue marlin, swordfish, yellowtail, salmon, or trout. In various aspects, the non-textured muscle tissue is combined with fat tissue. Sometimes, the muscle tissue and fat tissue are combined to create a surimi product. In certain occasions, the fish muscle and fat tissue is sushi-grade. The population of cells is isolated as embryonic stem cells, in some embodiments. In certain aspects, the population of cells has been modified to induce pluripotency. In many cases, the population of cells is isolated as multipotent adult stem cells. Culturing comprises growing and expanding the population of cells in cell culture, in various instances. Oftentimes, inducing differentiation comprises exposing the population of cells to culture conditions that stimulate differentiation. In some cases, differentiation comprises transdifferentiation of cells into a different cell type. Sometimes, inducing differentiation comprises exposing the population of cells to at least one growth factor that stimulates differentiation. In various aspects, culturing comprises growing the population of cells on a two dimensional surface. Oftentimes, culturing comprises growing the population of cells on a three-dimensional scaffold. In certain instances, culturing comprises growing the population of cells on micro-scaffolds within a bioreactor, wherein the micro-scaffolds enable cell adhesion. In some scenarios, the population of cells does not require an adherence substrate for survival and proliferation. Sometimes, the population of cells is adapted to suspension culture. In certain embodiments, the population of cells forms non-textured tissue after differentiation. The population of cells sometimes forms non-muscle tissue after differentiation. In certain cases, culturing comprises growing the population of cells in a media formulation comprising at least one nutritional supplement. In some instances, the at least one nutritional supplement comprises an omega-3 fatty acid. Typically, the at least one nutritional supplement comprises a polyunsaturated fatty acid. Sometimes, the at least one nutritional supplement comprises a monounsaturated fatty acid. Sometimes, the population of cells is cultured using a non-serum media formulation. In many cases, the population of cells is cultured using a mushroom-based media formulation.

Disclosed herein are methods of preparing foie gras, comprising cultured avian liver tissue. Some such methods comprise: a) obtaining a population of avian derived cells capable of self-renewal; b) differentiating the population of avian derived cells into hepatocytes; and c) inducing steatosis in the hepatocytes to generate cultured avian liver tissue having high lipid content; and d) preparing the cultured avian liver tissue as foie gras. Various aspects incorporate at least one of the following elements. Sometimes, the cells are duck cells. In certain aspects, the cells are goose cells. In some cases, differentiation comprises transdifferentiation of cells into a different cell type.

Disclosed herein are culinary foie gras compositions comprising tissue cultured hepatocytes having high lipid content and processed for human consumption. Various aspects incorporate at least one of the following elements. In some cases, the composition has been processed into a plurality of slices. In certain instances, each slice weighs no more than about 5 ounces. Each slice is oftentimes individually packaged. Sometimes, the foie gras composition weighs at least about 1.5 pounds, is round and firm, and has no blemish. In various aspects, the foie gras composition has a package label indicating an A grade rating. In certain embodiments, the foie gras composition weighs between about 0.75 to about 1.5 pounds. In some instances, the foie gras composition has a package label indicating a B grade rating. The foie gras composition weighs less than about 1 pound and has no more than three blemishes, in some cases. In some cases, the foie gras composition has a package label indicating a C grade rating. In certain embodiments, the tissue cultured hepatocytes are steatotic. In many instances, the tissue cultured hepatocytes are characterized by excess accumulation of cytoplasmic lipid droplets. The high lipid content is obtained by exposure to an exogenous compound that modulates at least one lipid metabolic pathway, in certain aspects. The high lipid content is often obtained by exposure to at least one of a toxin and a high lipid concentration. Sometimes, the high lipid content is obtained by modulation of at least one lipid metabolic pathway to enhance lipid retention within the population of cells. In certain instances, the high lipid content is obtained by alteration of at least one gene in the tissue cultured hepatocytes. Oftentimes, the foie gras composition further comprises cells having low lipid accumulation. In various aspects, the tissue cultured hepatocytes are differentiated from isolated embryonic stem cells. The tissue cultured hepatocytes are differentiated from induced pluripotent stem cells, in certain cases. The tissue cultured hepatocytes are differentiated from isolated multipotent adult stem cells, in certain instances. In some instances, the tissue cultured hepatocytes are generated by differentiation in a population of cells capable of self-renewal. Sometimes, differentiation comprises exposing the population of cells to culture conditions that stimulate differentiation. In some cases, differentiation comprises transdifferentiation of cells into a different cell type. In various cases, differentiation comprises exposing the population of cells to at least one growth factor that stimulates differentiation. Oftentimes, the tissue cultured hepatocytes are grown on a two dimensional surface. The tissue cultured hepatocytes are grown on a three-dimensional scaffold, in some instances. In various aspects, the tissue cultured hepatocytes are grown on micro-scaffolds within a bioreactor, wherein the micro-scaffolds enable cell adhesion. In certain embodiments, the tissue cultured hepatocytes do not require an adherence substrate for survival and proliferation. Sometimes, the tissue cultured hepatocytes are adapted to suspension culture. Sometimes, the tissue cultured hepatocytes form non-textured tissue. In various instances, the tissue cultured hepatocytes form non-muscle tissue. The tissue cultured hepatocytes are cultured in a media formulation comprising at least one nutritional supplement, in many cases. Sometimes, the at least one nutritional supplement comprises an omega-3 fatty acid. In certain embodiments, the at least one nutritional supplement comprises a polyunsaturated fatty acid. Sometimes, the at least one nutritional supplement comprises a monounsaturated fatty acid. Sometimes, the tissue cultured hepatocytes are cultured using a non-serum media formulation. In many cases, the tissue cultured hepatocytes are cultured using a mushroom-based media formulation.

Disclosed herein are compositions comprising cultured organ cells processed into a non-textured non-muscle food product for human ingestion. Various aspects incorporate at least one of the following elements. In some cases, the cultured organ cells comprise hepatocytes. In certain aspects, the cultured organ cells comprise avian cells. Oftentimes, the food product is processed into a plurality of slices. Sometimes, each slice weighs no more than about 5 ounces. Each slice is usually individually packaged. In many aspects, the food product is foie gras. The foie gras usually weighs at least about 1.5 pounds, is round and firm, and has no blemish. In certain instances, the foie gras has a package label indicating an A grade rating. In various aspects, the foie gras weighs between about 0.75 to about 1.5 pounds. The foie gras has a package label indicating a B grade rating, in certain embodiments. Sometimes, the foie gras weighs less than about 1 pound and has no more than three blemishes. In various instances, the foie gras has a package label indicating a C grade rating. Oftentimes, the tissue cultured hepatocytes are steatotic. In certain cases, the foie gras is characterized by high lipid content. The high lipid content is obtained by exposure to an exogenous compound that modulates at least one lipid metabolic pathway, in some aspects. The high lipid content is often obtained by exposure to at least one of a toxin and a high lipid concentration. In certain cases, the high lipid content is obtained by modulation of at least one lipid metabolic pathway to enhance lipid retention within the population of cells. Sometimes, the high lipid content is obtained by alteration of at least one gene in the tissue cultured hepatocytes. In some aspects, the foie gras composition further comprises cells having low lipid accumulation. In certain instances, the cultured organ cells are grown on a two dimensional surface. Oftentimes, the cultured organ cells are grown on a three-dimensional scaffold. The cultured organ cells are grown on micro-scaffolds within a bioreactor, wherein the micro-scaffolds enable cell adhesion, in various embodiments. The cultured organ cells sometimes do not require an adherence substrate for survival and proliferation. Sometimes, the cultured organ cells are adapted to suspension culture. In various aspects, the cultured organ cells form non-textured tissue. Sometimes, the cultured organ cells form non-muscle tissue. In various cases, the cultured organ cells are cultured in a media formulation comprising at least one nutritional supplement. Sometimes, the at least one nutritional supplement comprises an omega-3 fatty acid. The at least one nutritional supplement comprises a polyunsaturated fatty acid, in many instances. Sometimes, the at least one nutritional supplement comprises a monounsaturated fatty acid. Sometimes, the cultured organ cells are cultured using a non-serum media formulation. In many cases, the cultured organ cells are cultured using a mushroom-based media formulation.

Disclosed herein are edible foie gras compositions comprising cultured steatotic avian liver cells and seasoning. In some cases, the seasoning includes at least one of salt, pepper, and sugar.

Disclosed herein are foie gras compositions comprising cultured liver cells having high lipid content and liver cells having low lipid content. Various aspects incorporate at least one of the following elements. In some cases, the cultured liver cells having high lipid content and the liver cells having low lipid content are blended together. In certain instances, the foie gras composition is suitable as an ingredient for preparing one of a mousse, a parfait, and a pâté. Typically, the liver cells having low lipid content are cultured cells. In some embodiments, the liver cells having low lipid content are un-cultured cells.

Disclosed herein are edible compositions comprising avian liver cells grown in cell culture and processed for human consumption.

Disclosed herein are packaged foie gras compositions comprising cultured liver cells and packaging having a label indicating the foie gras composition was not produced by forced feeding.

Disclosed herein are packaged foie gras compositions comprising cultured liver cells processed into foie gras and packaging having a label indicating the foie gras was produced in a pathogen-free environment. In certain cases, the label indicates the composition was produced without exposure to avian bird flu virus.

Disclosed herein are packaged edible compositions comprising cultured cells processed into a food product and packaging having a label indicating the composition was produced without exposure to a toxin. In certain cases, the toxin is one of an insecticide, herbicide, and fungicide.

Disclosed herein are methods of producing cultured cells for human consumption without using antibiotics. Some such methods comprise: a) culturing a population of cells without using antibiotics; b) inducing differentiation within the population of cells; c) inducing high lipid accumulation within the population of cells; and d) processing the population of cells for human consumption. In some cases, differentiation comprises transdifferentiation of cells into a different cell type.

Disclosed herein are methods of producing cultured cells for human consumption without exposure to pathogens. Some such methods comprise: a) culturing a population of cells in a pathogen-free culture environment; b) inducing differentiation within the population of cells; c) inducing high lipid accumulation within the population of cells; and d) processing the population of cells for human consumption. In some cases, differentiation comprises transdifferentiation of cells into a different cell type.

Disclosed herein are methods of producing cultured cells for human consumption without exposure to toxins. Some such methods comprise: a) culturing a population of cells in a toxin-free culture environment; b) inducing differentiation within the population of cells; c) inducing high lipid accumulation within the population of cells; and d) processing the population of cells for human consumption. In some cases, differentiation comprises transdifferentiation of cells into a different cell type.

Disclosed herein are methods of producing cultured non-textured tissue having high lipid content and no vasculature. Some such methods comprise: a) culturing a population of cells; b) inducing differentiation in the population of cells; c) manipulating lipid metabolic pathways to induce steatosis in the population of cells such that the cells accumulate high lipid content; and d) processing the population of cells into non-textured tissue having no vasculature. In some cases, differentiation comprises transdifferentiation of cells into a different cell type.

Disclosed herein are methods of producing cultured tissue having increased nutritional content for human consumption. Some such methods comprise: a) culturing a population of cells in a culture medium having at least one nutritional supplement; b) manipulating lipid metabolic pathways to induce steatosis in the population of differentiated cells such that the cells accumulate high lipid content; and c) processing the population of differentiated cells into non-textured tissue having no vasculature for human consumption. Various aspects incorporate at least one of the following elements. In some cases, the at least one nutritional supplement comprises an omega-3 fatty acid. Oftentimes, the at least one nutritional supplement comprises a polyunsaturated fatty acid. Sometimes, the at least one nutritional supplement comprises a monounsaturated fatty acid.

Disclosed herein are methods of producing cultured organ tissue for human consumption. Some such methods comprise: a) culturing a population of cells capable of self-renewal; b) inducing differentiation in the population of cells to generate organ tissue; and c) processing the organ tissue for human consumption. Various aspects incorporate at least one of the following elements. In some cases, the organ tissue is liver, heart, kidney, stomach, intestine, lung, diaphragm, esophagus, thymus, pancreas, or tongue tissue. In some cases, differentiation comprises transdifferentiation of cells into a different cell type. In various embodiments, the organ tissue is liver tissue. Sometimes, processing comprises blending the organ tissue with additional cellular tissues. The additional cellular tissues comprise non-steatotic liver cells, in many instances.

Disclosed herein are methods of producing cultured fish tissue having enhanced nutritional content for human consumption. Some such methods comprise: a) culturing a population of fish myocytes in a culture media having at least one nutritional supplement; b) expanding the population of myocytes; and c) processing the population of myocytes into fish tissue for human consumption. Various aspects incorporate at least one of the following elements. In some cases, the fish tissue comprises fast twitch muscle fibers. In certain embodiments, the method further comprises combining the population of myocytes with a population of adipocytes. The fish myocytes are often salmon myocytes. The fish myocytes are sometimes tuna myocytes. In some cases, the fish myocytes are trout myocytes.

Disclosed herein are edible compositions comprising fish tissue produced from cultured myocytes and adipocytes according to any of the methods described herein.

Disclosed herein are bioreactor systems for producing cultured tissues suitable for human consumption comprising: a) a reactor chamber comprising a plurality of micro-scaffolds that provide adhesion surfaces for cellular attachment; b) a population of self-renewing cells cultivated within bioreactor; c) a first source providing at least one maintenance media comprising components for maintaining the population of self-renewing cells without spontaneous differentiation; and d) a second source providing at least one differentiation media comprising components for differentiating the population of self-renewing cells into a specific lineage; wherein the reactor chamber receives maintenance media from the first source to cultivate the population of cells and receives differentiation media from the second source to differentiate the population of cells, wherein the population of cells generated in a single batch comprises cultured tissues suitable for human consumption and having a dry weight of at least 1 kg. Various aspects incorporate at least one of the following elements. In some cases, the system further comprises at least one sensor for monitoring the reactor chamber. In certain embodiments, the at least one sensor is a biosensor, a chemosensor, or an optical sensor. Oftentimes, the at least one sensor is configured to monitor at least one of pH, temperature, oxygen, carbon dioxide, glucose, lactate, ammonia, hypoxanthine, amino acid(s), dopamine, and lipid(s). Sometimes, the system further comprises at least one additional reactor chamber. The single batch often has a dry weight of at least 5 kg. Sometimes, the bioreactor system further comprises a plurality of micro-scaffolds. Alternatively, the bioreactor system further comprises at least one 3D scaffold. The bioreactor system frequently comprises a third source providing at least one steatotic media comprising components for inducing steatosis or lipid accumulation in the population of cells. In various cases, the population of cells is cultured in media comprising at least one nutritional supplement. Sometimes, the population of cells is cultured using a non-serum media formulation. In many cases, the population of cells is cultured using a mushroom-based media formulation.

Disclosed herein, in some aspects, are cultured food products for human consumption, comprising the cultured tissue produced according to the methods of any one of the foregoing methods. Sometimes, the cultured food product comprises packaging having a label indicating the cultured tissue was produced in a pathogen-free environment, a toxin-free environment, without force-feeding an animal, or any combination thereof. In certain instances, the cultured tissue is processed into a plurality of slices and packaged to form the cultured food product.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 shows a flow chart for a process of producing cultured cells having lipid accumulation for human consumption.

FIG. 2 shows a flow chart for a process of producing cultured meat comprising myocytes and adipocytes for human consumption.

FIG. 3 shows an overview of an exemplary process for culturing meat.

FIG. 4A shows a diagram illustrating methods of generating steatotic hepatocytes for producing cultured foie gras. FIG. 4B shows a diagram illustrating methods of producing cultured fish tissue for consumption.

FIG. 5A shows isolated trout myosatellite cells. FIG. 5B shows expression of genetic markers in the isolated trout myosatellite cells. FIG. 5C shows mature myotubes formed by differentiating the myosatellite cells. FIG. 5D shows a sheet of myotubes following differentiation from the myosatellite cells.

FIG. 6A shows a co-culture of salmon myosatellite cells (arrowheads) and salmon pre-adipocytes (arrows). The pre-adipocytes can be differentiated into adipocytes, and the myosatellite cells differentiated into myocytes (arrowhead) as shown in FIG. 6B.

FIG. 7A shows salmon fibroblasts induced to form spheroids for propagation in a bioreactor. FIG. 7B shows confirmation of the viability of these spheroids upon returning them to 2-D culture conditions and observing that the fibroblasts migrated circumferentially to form colonies.

FIG. 8 shows a successful cell culture of bass myosatellite cells.

FIG. 9 shows a spheroid formed from duck hepatocytes growing in a hanging drop and a spinner flask into which the spheroid can be transferred for 3-dimensional suspension culture.

FIG. 10A shows duck hepatocytes growing in culture following successful differentiation.

FIG. 10B confirmed successful hepatocyte differentiation by measuring markers of hepatocyte differentiation.

FIG. 11A shows self-renewing duck cells generated by culturing primary fibroblasts and harvesting colonies of dividing cells. FIG. 11B shows trout self-renewing cells generated by culturing primary fibroblasts and harvesting colonies of dividing cells.

FIG. 12 shows an exemplary embodiment of a genetic construct that can be introduced into a cell to provide inducible differentiation into a hepatocyte.

FIG. 13 shows an exemplary embodiment of a construct that can be introduced into a cell to allow inducible expression of one or more genes that predispose the cell to steatosis.

FIG. 14 shows an exemplary embodiment of a DNA construct system that can be introduced into a cell to allow a proliferation/differentiation switch from a pluripotent phenotype into a differentiated phenotype.

FIG. 15 shows an exemplary construct that can be introduced into a cell to provide an inducible “off-switch”.

FIG. 16A shows successful induction of steatosis in duck hepatocytes upon incubation with linoleic acid. FIG. 16B shows a dose response curve correlating the percentage of steatotic hepatocytes with the concentration of linoleic acid.

FIG. 17 shows the hepatocyte population size when cultured in the media having progressively decreasing concentrations of fetal bovine serum (FBS) in the presence of soybean hydrolysate.

FIG. 18 shows duck fibroblasts that have also been successfully grown in 10% shiitake mushroom extract after successive reduction of fetal bovine serum from the cell culture media.

FIG. 19A shows duck fibroblasts grown in serum-free media without additional supplementation; FIG. 19B shows a control culture grown in DMEM supplemented with 10% fetal bovine serum.

FIG. 20 shows a diagram of a bioreactor system for culturing cells for human consumption.

FIG. 21 shows another diagram of a bioreactor system being used as part of a meat production process.

FIG. 22A shows an embryoid body generated using the hanging drop method (left panel) and an exemplary bioreactor that the embryoid bodies are transitioned to for growing in 3-D culture. FIG. 22B shows another exemplary bioreactor (left panel) and cells from the spheroids that are propagated in the 3-D culture (right panel).

FIG. 23A shows trout myotubes that have successfully differentiated from myosatellite cells attached to glucomannan microscaffolds. FIG. 23B shows a negative control of undifferentiated myosatellite cells from the same preparation grown in identical cell culture conditions.

FIG. 24A shows duck fibroblasts (arrowheads) successfully grown on glucomannan microscaffolds (arrows). FIG. 24B shows a representative glucomannan microscaffold.

FIG. 25 shows a still image captured from a video of duck muscle tissue demonstrating spontaneous contraction.

FIG. 26 shows duck liver pâté and foie gras butter made using duck steatotic liver cells.

FIG. 27 shows salmon pâté and duck meat pâté prototypes made according to the methods described herein.

FIG. 28A shows an exemplary embodiment of a method of Cre delivery for the purpose of activating/silencing particular genes. FIG. 28B shows different methods of using Cre to induce a “switch” between activated gene sets relevant to meat creation (e.g., proliferation and differentiation).

### DETAILED DESCRIPTION OF THE INVENTION

Disclosed herein are systems and methods for producing food products using cellular agriculture. Cell cultured food products provide many advantages that obviate or greatly reduce the negative impacts caused by traditional food production. These advantages are particularly felt in the area of meat production, which are typically produced using intensive livestock production or through fishing and fisheries. Instead of raising or catching live animals and fish to be harvested for their meat, cells having self-renewal capacity are isolated or created and grown in cell culture. In some cases, the cells are naturally capable of self-renewal such as embryonic stem cells and pluripotent progenitor cells. Alternatively, or in combination, the cells are manipulated to acquire the ability to self-renew. These cells are cultured and expanded to a desired quantity. Oftentimes, the cells are cultured in a scalable manner, for example, using bioreactors that enable large-scale production. Various media formulations are optionally used to enable the maintenance of the capacity for self-renewal such as during expansion of the cell population, or to push the cells down certain differentiation pathways to generate the desired cell type. For example, in some instances, the cultured cells are induced to differentiate into muscle cells, adipose cells, or organ cells. In some cases, differentiation comprises transdifferentiation of cells into a different cell type. For instance, immortalized fibroblasts can be expanded and then transdifferentiated into myocytes, adipocytes, hepatocytes, and/or other desired cell types. Sometimes, the media formulations are modified from conventional media to not require fetal bovine serum or serum alternatives, which remain untested for human consumption. Media formulations can include low-serum or no-serum formulations that are derived from plants to reduce or obviate the use of animal components such as fetal bovine serum. Examples of plant-based formulations include soybean-based and plant hydrolysate-based media formulations. Media formulations often comprise at least one mushroom-based ingredient. In certain cases, the at least one mushroom-derived extract replaces fetal bovine serum in the media formulation. Some media formulations comprise at least one ingredient for enhancing the nutritional content of the cultured cells. Alternatively, co-culture systems are used to provide conditioned media systems that increase efficiency by obviating the need for recombinant protein production and allowing the culture media to be recycled. In addition to alternative media formulations, three-dimensional scaffolding and tissue engineering platforms are used to facilitate large-scale growth, in many cases. Oftentimes, scalable bioreactors provide the requisite growth needed for mass production. In some instances, three-dimensional scaffolding is used to provide structural support and guide the growth of the cultured cells into the desired structure and/or texture analogous with the equivalent food product produced using conventional methods. Alternatively or in combination, micro-scaffolds enable the growth of adherent cells in suspension culture such as in a bioreactor. These micro-scaffolds can be engineered to enhance stem cell proliferation, direct cell differentiation into the relevant lineage, and modulate flavor, texture, and tensile elasticity of the final meat product. Some adherent cells are modified to grow in suspension culture without requiring an adherent surface. Certain food products are produced using a homogeneous population of cells such as, for example, liver cells for making foie gras. Alternatively, some food products are produced using a heterogeneous population of cells such as a combination of muscle and fat cells. In some cases, a population of cells is differentiated into multiple cell types to create a heterogeneous population of differentiated cells. Alternatively, independent cell populations are differentiated into distinct cell types and subsequently combined. These methods using heterogeneous cell populations enable the production of certain tissues such as, for example, salmon meat that is composed of a combination of muscle and fat cells. Oftentimes, the cultured cells are modified to produce a desired cell or tissue phenotype. Cultured calls can be modified with one or more genetic constructs to confer a desired phenotype such as a state of self-renewal, differentiation into a cell or tissue type, or predisposition to steatosis. Cultured cells can also be modified through adjustments to the culture environment. For example, liver cells are optionally cultured in lipid rich media to induce steatosis via excess lipid uptake and storage inside the cytoplasm. In many cases, the steatotic liver cells are harvested and processed as foie gras or a foie gras food product. Harvested cells are typically processed to produce a desired consistency and/or texture. In some cases, the harvested cells are processed to achieve particular tastes, textures and other culinary properties that are indistinguishable from the high quality meats they are intended to reproduce.

The systems and methods for producing cell cultured food products disclosed herein provide numerous advantages. The cultured meat is not exposed to pathogens such as avian bird flu or various bacterial strains during production. Likewise, the systems and methods disclosed herein can provide for meat production without the use of antibiotics. This has the benefit of not inadvertently exposing humans to antibiotics while also avoiding the increased risk of bacteria developing antibiotic resistance. In addition, cultured food production does not require feed crops and avoids the production of animal waste, which often contains fecal coliform bacteria, ammonia, and phosphorus. For example, a substantial amount of land is devoted to growing feed crops for livestock, which entails the widespread use of fertilizer, pesticides, and herbicides. By contrast, cultured food products are capable of being produced with a relatively small environmental footprint.

Non-textured tissues such as foie gras and certain fish meats such as salmon are produced using various systems and methods described herein. Some methods enable production of cultured non-textured tissue having high lipid content such as steatotic hepatocytes useful for making foie gras. Such methods often comprise: a) obtaining a population of differentiated cells capable of self-renewal; b) culturing the population of differentiated cells; c) manipulating at least one lipid metabolic pathway to induce steatosis in the population of differentiated cells such that the cells accumulate high lipid content; and d) processing the population of differentiated cells into non-textured tissue. In some cases, the differentiated cells capable of self-renewal are obtained through transdifferentiation (e.g., direct cell reprogramming). Sometimes, methods described herein produce cultured organ tissue for human consumption. Such methods comprise: a) culturing a population of cells capable of self-renewal; b) inducing differentiation in the population of cells to generate organ tissue; and c) processing the organ tissue for human consumption.

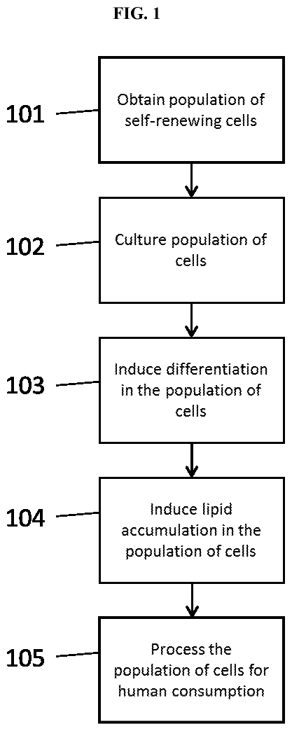

FIG. 1 illustrates one embodiment of a process for culturing cells for human consumption. In this example, a population of self-renewing cells is obtained 101. As described herein, self-renewing cells are sometimes embryonic stem cells, induced pluripotent stem cells, embryonic germ cells, immortalized differentiated cells, or nascent adult stem cells. The population of cells is cultured 102, and typically expanded to the desired population size. Next, differentiation is induced in the population 103. In some cases, differentiation comprises transdifferentiation of cells into a different cell type. In this example, cells in the population are differentiated into hepatocytes. Oftentimes, lipid accumulation is induced in the population of cells comprising differentiated hepatocytes 104. Finally, the population of cells is processed for human consumption 105. For example, hepatocytes are often processed into foie gras or a foie gras food product.

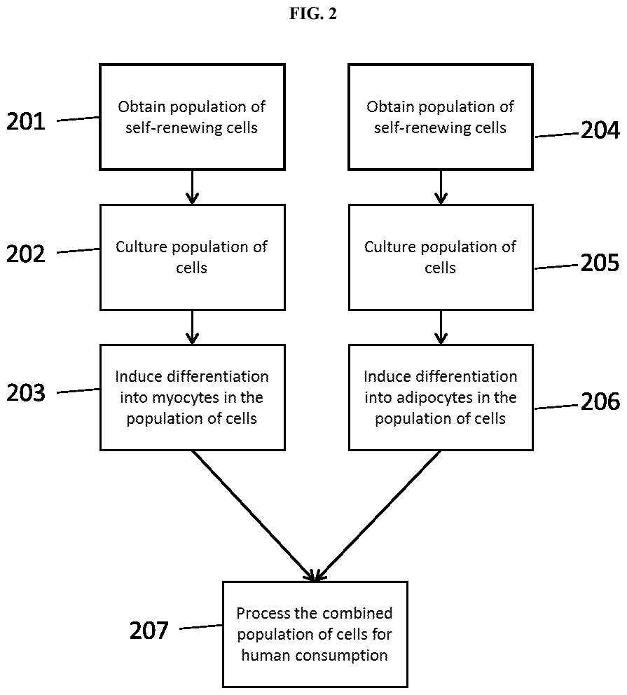

FIG. 2 illustrates one embodiment of a process for culturing muscle tissue for human consumption. In this example, a first and a second population of self-renewing cells are obtained 201, 204. The two populations of cells are cultured 202, 205, and typically expanded to the desired population size. Next, differentiation is induced in the two populations 203, 206. In this case, the differentiation into myocytes is induced in the first population of cells 203. In some cases, differentiation comprises transdifferentiation of cells into a different cell type. Differentiation into adipocytes is induced in the second population of cells 206. Finally, the two populations of cells are processed for human consumption 207. In this case, the first and second populations are combined and processed into meat comprising both muscle and fat cells for human consumption.

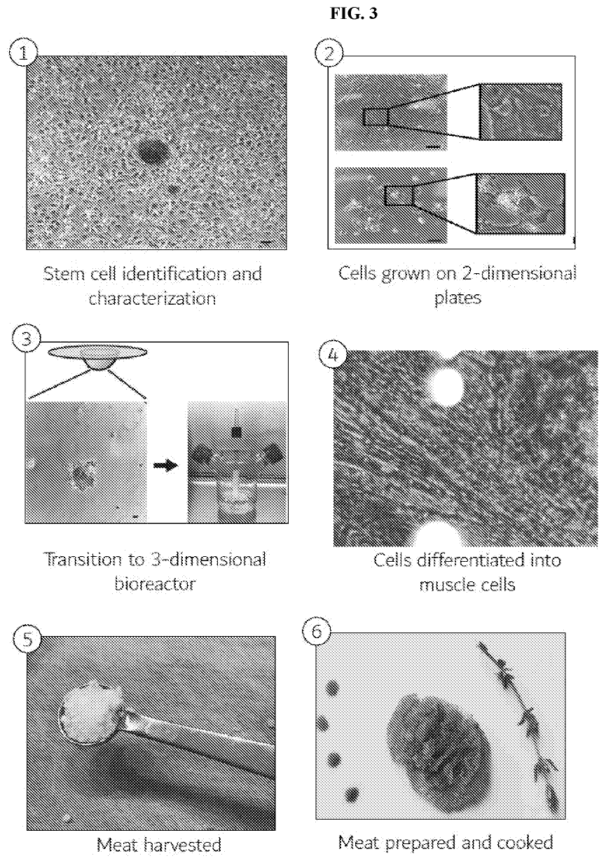

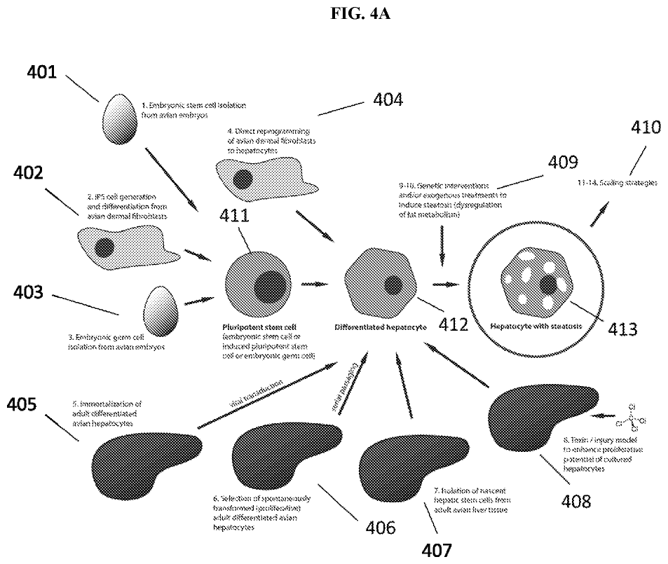

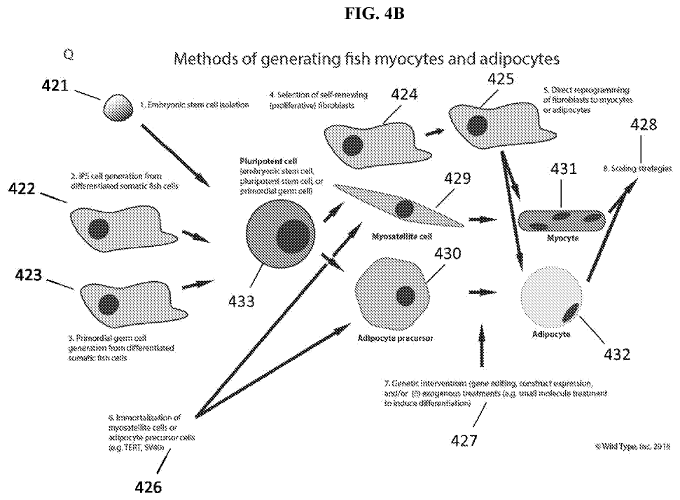

An overview of an exemplary process for preparing cultured meat for consumption is shown in FIG. 3. First, stem cell identification, isolation, and characterization are carried out. These cells are initially grown in two-dimensional culture such as on a feeder cell layer. The cells are eventually transitioned into suspension culture in a bioreactor allowing for larger-scale cell growth. Subsequent to transitioning to suspension culture, the cells are differentiated into muscle cells. In some cases, differentiation comprises transdifferentiation of cells into a different cell type (e.g., from immortalized fibroblasts into muscle and/or fat cells). The meat is then harvested, and finally prepared and cooked. Various approaches can be used to obtain cell lines suitable for preparing cultured food products (FIGS. 4A-4B).

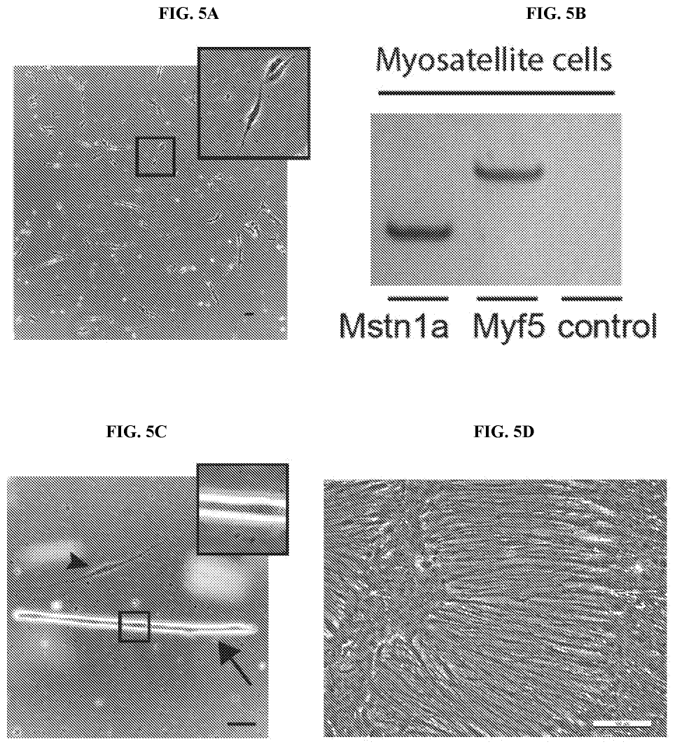

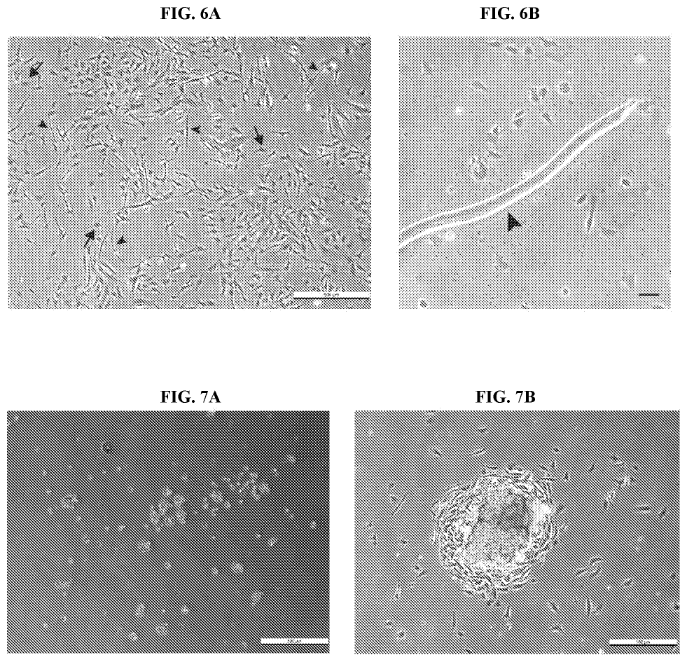

Disclosed herein, in certain aspects, are methods of producing synthetic food products comprising tissue derived from fish. In some embodiments, fish myocytes and adipocytes are utilized for development of fish-related foods based on their intrinsic regenerative capacity during early developmental stages. In an exemplary embodiment of this process, trout pre-adipocytes and myosatellite cells (capable of differentiating into myocytes) were isolated, cultured, and characterized. Trout myosatellite cells were isolated and then characterized as shown in FIGS. 5A-5D. Where present, insets magnify image details, and the scale bar is equal to 10 μm in all micrographs unless otherwise indicated. Substantially pure populations of piscine myosatellite cells were successfully isolated and are shown in FIG. 5A with the myosatellite cells making up about 80% of the isolated cells. RT-PCR analysis of these isolated cells revealed expression of the transcription markers Mstn1a and Myf5 which are markers of pluripotency (FIG. 5B). Next, culture conditions were optimized for these cells. Culture media protocols were used to successfully differentiate myosatellite cells (arrowhead) into mature, differentiated myocytes (arrow) (FIG. 5C). The resulting sheets of trout myotubes differentiated from the myosatellite cells are shown in FIG. 5D (scale bar is 100 μm). In some embodiments, myosatellite cells can be co-cultured with pre-adipocytes. In an exemplary embodiment, salmon myosatellite cells (arrowheads) were co-cultured with salmon pre-adipocytes (arrows) for producing a food product comprising both muscle and fat cells or tissue as shown in FIG. 6A (scale bar is 100 μm). The pre-adipocytes were differentiated into adipocytes, and the myosatellite cells differentiated into myocytes (arrowhead) as shown in FIG. 6B (scale bar is 10 μm).

In some cases, salmon fibroblasts are used for producing a food product. Salmon fibroblasts can be induced to form spheroids for propagation in a bioreactor (FIG. 7A) (scale bar is 100 μm). The viability of these spheroids is confirmed by returning them to 2-Dimensional culture conditions and observing that the fibroblasts migrated circumferentially to form colonies (FIG. 7B) (scale bar is 100 μm). In some cases, the fibroblasts are propagated, and then transdifferentiated into desired cell types such as myocytes, adipocytes, hepatocytes, or any combination thereof.

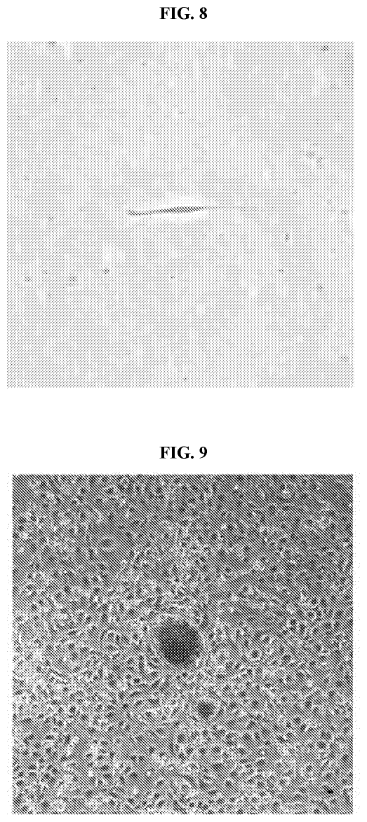

The methods disclosed herein are capable of being applied to various aquatic species. For example, cell cultures of bass myosatellite cells have also been successfully cultivated utilizing standard cell culture protocols (FIG. 8). In certain embodiments, disclosed herein are meat products comprising cells or tissue derived from one or more types of aquatic organisms. Sometimes, an aquatic organism is selected from the group consisting of sea bass, tuna, mackerel, blue marlin, swordfish, yellowtail, salmon, trout, eel, abalone, squid, clams, ark shell, sweetfish, scallop, sea bream, halfbeak, shrimp, flatfish, cockle, octopus, or crab. In certain cases, an aquatic organism is a type of fish selected from the group consisting of sea bass, tuna, mackerel, blue marlin, swordfish, yellowtail, salmon, trout, or flatfish. In some instances, the aquatic organism can be a round fish or flat fish. A round fish can include bass, catfish, Arctic char, cod, haddock, herring, sardines, tilapia, trout, red snapper, salmon, swordfish, and tuna. A flat fish can include flounder, sole, halibut, and turbot. Varieties of tuna include yellowfin, southern Bluefin, northern Bluefin, Thunnus alalunga, Thunnus atlanticus, and Thunnus obesus. Varieties of salmon include Atlantic salmon, sockeye salmon, Chinook salmon (also called king salmon), Coho salmon, chum salmon, and pink salmon. Varieties of trout include rainbow trout, cutthroat trout, brown trout, red mountain trout, brook trout, and lake trout.

Disclosed herein, in certain aspects, are methods of producing synthetic food products comprising tissue derived from avian species. In some embodiments, the synthetic food product comprises avian hepatocytes and/or liver tissue derived from a duck or goose. In some embodiments, the synthetic food product comprises steatotic liver tissue. In some embodiments, these methods utilize self-renewing cells (e.g. pluripotent or multipotent cells) for development of avian-related foods based on their intrinsic regenerative capacity during early developmental stages. Successfully isolated duck embryonic stem cells growing in culture are shown in FIG. 9.

### Cell Lines

Some systems and methods disclosed herein comprise generation of cell line(s) capable of self-renewal for cultured food production. In one approach, embryonic stem cells are isolated. Embryonic stem cells are pluripotent stem cells generated from early-stage embryos. Typically, the embryonic stem cells are harvested from a blastocyst 4-5 days after fertilization has occurred. The blastocyst has an inner cell mass that is removed and placed in culture. Those cells that remain viable in cell culture conditions are used to establish a cell line capable of self-renewal. For example, FIG. 4A illustrates certain approaches for generating steatotic hepatocytes. In some instances, the embryonic stem cells are obtained from avian embryos such as, for example, duck or goose embryos 401. These duck or goose embryonic stem cells are pluripotent stem cells 411 optionally used for producing cultured foie gras. Sometimes, avian embryonic stem cells are isolated from blastodermal cells in Eyal-Giladi and Kochav Stage 10 (EGK-X) avian embryos. For example, avian embryonic stem cells can be isolated by culturing them on inactivated STO feeders cells in an embryonic stem cell medium (ESA) with certain growth factors such as bFGF, IGF-1, mSCF, IL-6, OSM, LIF, IL-6, and IL-11 as described in Aubel P., Pain B. Chicken embryonic stem cells: establishment and characterization. Methods Mol. Biol. 2013; 1074:137-150. Successfully isolated duck embryonic stem cells growing in culture are shown in FIG. 9. These approaches can also be utilized for generating fish myocytes and/or adipocytes (FIG. 4B).

Once isolated, the embryonic stem cells are usually maintained in an undifferentiated state. Sometimes, published protocols are modified to maintain the embryonic stem cells in an undifferentiated state. Modifications to published protocols can include the use of optimized matrix substrates and the use of optimized media formulations to achieve persistent cellular proliferation and maintenance of a de-differentiated state. In some cases, avian embryonic stem cells are maintained in the undifferentiated state using leukemia inhibitory factor (LIF), a member of the interleukin-6 family of cytokines as described in Horiuchi et a., Chicken leukemia inhibitory factor maintains chicken embryonic stem cells in the undifferentiated state. J. Biol. Chem. 2004; 279: 24514-24520. Alternatively, avian embryonic stem cells are maintained in an undifferentiated state without requiring the use of LIF in the culture media. In certain instances, the avian embryonic stem cells are maintained in an undifferentiated state in a culture media formulation containing LIF without the use of other cytokines or feeder cells. A media formulation sometimes comprises recombinant LIF. In some cases, recombinant LIF is produced as a fusion protein with an affinity tag for purification. Typically, a fusion protein with an affinity tag for purification uses at least one affinity tag selected from glutathione S-transferase (GST), FLAG tag, S-tag, heavy chain of protein C (HPC), streptavidin binding peptide, streptavidin, streptavidin tag, histidine affinity tag, polyhistidine tag, polycysteine tag, polyaspartate tag, albumin-binding protein (ABP), calmodulin binding peptide, cellulose binding domain, chitin binding domain, and choline binding domain. In some instances, an affinity purified fusion protein is cleaved or digested to remove the affinity tag.

The isolated embryonic stem cells are typically differentiated into a desired cell type. The desired cell type is usually the fully differentiated cell that makes up the food product or a portion of the food product. Sometimes, a differentiated cell is a hepatocyte or liver cell 412. FIG. 10A shows duck hepatocytes growing in culture following successful differentiation. Differentiation was confirmed by measuring markers of hepatocyte differentiation (L-FABP, alpha-fetoprotein, and HNF3b, with beta-actin as a loading control) using RT-PCR (FIG. 10B). As shown in FIG. 10B, hepatocytes (right lane) generates observable expression of the hepatocyte differentiation markers compared to a lack of expression in the control undifferentiated cells (left lane). Cultured hepatocytes are often used to generate foie gras. In some instances, a differentiated cell is a myocyte or skeletal muscle cell. A differentiated cell is often an adipocyte that is optionally used in combination with other cell types such as myocytes for production of cultured meat products having both fat and muscle tissue. For example, salmon myocytes and adipocytes are sometimes used to produce sushi grade salmon meat for human consumption. Oftentimes, differentiated cells are organ cells such as, for example, striated or skeletal muscle cells, smooth muscle cells, cardiac muscle cells, spleen cells, thymus cells, endothelial cells, blood cells, bladder cells, liver cells, kidney cells, pancreas cells, lung cells, or any combination thereof. Alternatively, the desired cell type is sometimes an intermediate cell type such as an adult stem cell or progenitor cell useful for generating the fully differentiated cell type. Oftentimes, differentiation is achieved by optimization of standard protocols such as described in the website www.abcam.com/protocols/hepatocyte-differentiation-protocol. For example, embryonic stem cells and induced pluripotent stem cells are both capable of being differentiated into hepatocytes by splitting the cells into Matrigel coated plates in mTESR media with ROCK inhibitor Y27632, treating with definitive endoderm (DE) media, followed by hepatic endoderm (HE) media, immature hepatocyte (IMH) media, and finally mature hepatocyte (MH) media. Some media formulations are modified to enhance proliferation, differentiation, or other desired qualities in the cultured cells.

Some methods provide for generation of induced pluripotent stem cells 402 used to produce the food products disclosed herein. Sometimes, an episomal reprogramming strategy is employed to create induced pluripotent stem cells (iPSC) from fibroblasts, using an episomal reprogramming strategy optimized from the approach reviewed in Drozd et al., Generation of human iPSCs from cells of fibroblastic and epithelial origin by means of the oriP/EBNA-1 episomal reprogramming system. Stem Cell Research & Therapy. 2015; 6:122. For example, in some instances, at least one episomal vector expressing a combination of reprogramming factors such as Oct3/4, Sox2, Klf4, L-Myc, C-Myc, Lin28, Nanog, and Lin4 are introduced cells originating from adult avian fibroblasts. Additional factors that are sometimes added include p53 for overcoming reprogramming barriers such as cellular senescence. As an example, an (ori-P/EBNA-1)-based episomal vector enables reprogramming while persisting episomally inside of the reprogrammed cells. Episomal reprogramming provides an approach that precludes the generation of a “genetic footprint” because the episomal approach generates iPSC lines without integration of reprogramming vectors that result from classical viral reprogramming strategies. Finally, in some cases, differentiation of iPSCs is achieved using optimized standard protocols as described elsewhere herein for embryonic stem cells.

In some cases, embryonic germ cells are used as a source of pluripotent stem cells capable of self-renewal 403. Embryonic germ cells are capable of differentiation into the desired cell type such as, for example, mature hepatocytes for the purpose of liver tissue production. Sometimes, embryonic germ cells are isolated using protocols such as described in Guan et al., Derivation and characteristics of pluripotent embryonic germ cells in duck. Poultry Science. 2010; 89(2): 312-317. For example, duck embryonic tissue at stage 28 is obtained and subsequently dissociated using trypsin. The dissociated cells are harvested by centrifugation and then cultured in suspension culture in the presence of stem cell factor (SCF), leukemia inhibitory factor (LIF), and basic fibroblast growth factor (FGF). The embryonic germ cells typically form colonies, which are then reseeded into plates with feeder cells. In some instances, the isolated embryonic germ cells are expanded and optionally differentiated into the desired cell type for food production as described herein.

In some cases, differentiated cells are reprogrammed into the desired cell type without creating an intermediate pluripotent cell type 404. This process is sometimes referred to as transdifferentiation in which the desired cell type is generated from a non-stem cell. Sometimes, this method is carried out as described in Simeonov K P and Uppal H, Direct reprogramming of human fibroblasts to hepatocyte-like cells by synthetic modified mRNAs. PLOS ONE. 2014; 9(6): e100134. Oftentimes, isolated fibroblasts are reprogrammed into hepatocytes or hepatocyte-like cells by culturing the fibroblasts in an optimized hepatic growth media while expressing at least one factor of FOXA1, FOXA3, HNF1A, and HNF4A. In some cases, HNF1A and at least two of FOXA1, FOXA3, and HNF4A are expressed in the fibroblasts to convert them into hepatocytes. Oftentimes, expression or overexpression of any of the foregoing factors into differentiated cells for reprogramming is obtained by introducing exogenous DNA or RNA into the cells using genetic techniques known in the art. Alternatively, the isolated fibroblasts are reprogrammed into myocytes or adipocytes. In some cases, the reprogramming entails transdifferentiation of the isolated cells such as fibroblasts into a different cell type. For example, reprogrammed salmon myocytes and adipocytes are useful for producing edible salmon meat such as salmon grade sushi.

Sometimes, fully differentiated cells of the desired cell type are immortalized to generate a cell line capable of self-renewal. For example, myocytes, adipocytes, and/or hepatocytes can be immortalized for purposes of food production. Oftentimes, a classically-defined immortalization strategy using transformation is applied to differentiated adult cells to generate cell lines with indefinite proliferative capacity 405. In various cases, cell immortalization is achieved by artificial expression of key proteins required for immortality. In some examples, differentiated adult cells are immortalized via expression or overexpression of at least one of SV40 Large T Antigen, hTERT, HPV E6/E7, EBV, MycT58A, RasV12, and p53. In some cases, avian hepatocytes are immortalized to generate an adult avian hepatocyte cell line capable of self-renewal. Such a cell line aids in the large-scale production of hepatocyte-based food products such as foie gras. Sometimes, salmon myocytes and/or adipocytes are immortalized to generate adult salmon myocyte and adipocyte cell lines capable of self-renewal. Such immortalized cell lines allow for large-scale production of salmon meat such as sushi grade salmon. In some cases, differentiated cell lines (e.g., fibroblasts) are immortalized and subsequently transdifferentiated into a desired cell type such as myocytes, adipocytes, hepatocytes, or any combination thereof, and can be used to generate various types of food products such as fish meat or avian liver.

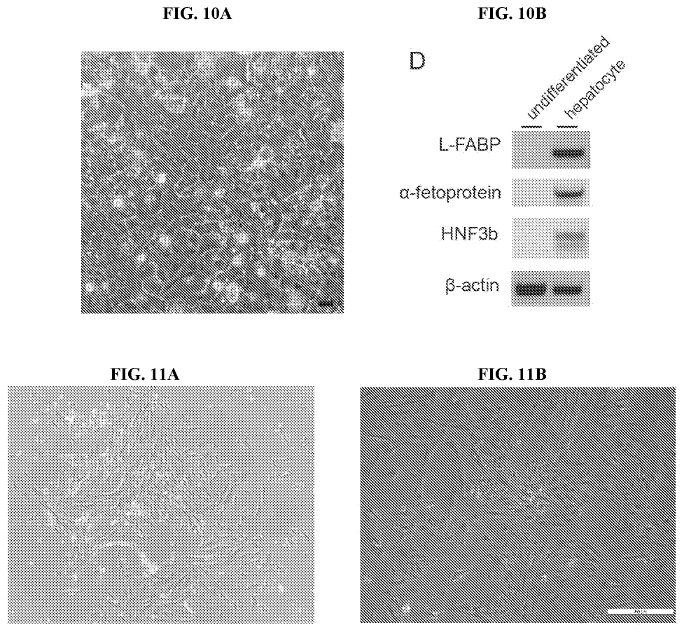

In some cases, an immortalized cell line capable of self-renewal is generated without transformation or direct genetic modification 406, in certain instances. Under this approach, a cell population is typically harvested and sequentially passaged for weeks until most cells undergo senescence, while a few spontaneous mutations arise that lead to the generation of a cell line with indefinite replicative potential. In some cases, the cell population is obtained from embryonic differentiated cells such as, for example, embryonic (differentiated) liver cells. This process is capable of being applied to avian cells to generate immortalized avian hepatocytes such as described in Lee et al., Establishment of an immortal chicken embryo liver-derived cell line. Poultry Science. 2013; 92(6):1604-12. This method obviates the need for integrating viruses in producing an immortalized cell line without the use of exogenous genetic material or genetic manipulation. For example, FIG. 11A shows duck self-renewing cells generated by culturing primary fibroblasts and harvesting colonies of dividing cells after 6-8 weeks. FIG. 11B shows trout self-renewing cells generated by culturing primary fibroblasts and harvesting colonies of dividing cells after 6-8 weeks. The self-renewing cells were then characterized for morphology, proliferation rate, and proliferative capacity (e.g. number of passages achieved without changes in morphology, proliferative rate, and without genomic instability). In some cases, differentiated cell lines (e.g., fibroblasts) are immortalized and subsequently transdifferentiated into a desired cell type such as myocytes, adipocytes, hepatocytes, or any combination thereof, and can be used to generate various types of food products such as fish meat or avian liver.

In some cases, nascent adult stem cells capable of self-renewal are isolated. For example, the liver is one of the few organs with regenerative capacity in adult mammalian and avian organisms. The existence of stem cells within adult hepatic tissue is reviewed in Navarro-Alvarez et al., Hepatic stem cells and liver development. Methods Mol Biol. 2010; 640:181-236. Accordingly, in some instances, nascent hepatic stem cells are isolated, cultivated, and expanded for use in cultured food production 407. Sometimes, fish pre-adipocytes and satellite cells are isolated and cultured to form cell lines suitable for expansion and differentiation into adipocytes and myocytes, respectively. The differentiated adipocytes and myocytes are usually then co-cultured together at a certain ratio to produce a desired final composition of adipocytes and myocytes in the resulting food product.

In some cases, liver cells are treated with toxic chemical compounds to generate cells with enhanced proliferative capacity 408. For example, such exposure to toxic compounds has been shown to elicit a proliferative response within the liver parenchyma. Accordingly, these liver cells with enhanced proliferative capacity are cultivated and expanded for use in cultured food production, in various instances.

In some cases, cells obtained using any of the foregoing methods is further modified to generate a cell line that does not require an adherent substrate for growth or survival. This method sometimes involves the creation of hepatocytes that do not require an extracellular matrix for attachment in order to survive and proliferate. Advantages of being able to grow cells in suspension culture include the ability to easily and rapidly scale up growth. Oftentimes, suspension cultures are less labor and/or resource intensive because they are able to culture cells based on volume rather than surface area and allow for passaging of cells without detachment steps such as trypsinization. A cell line that does not require an adherent substrate is often combined with a bioreactor cell culture system to enhance large-scale food production. In certain cases, stem cells are adapted to suspension culture, allowing for expansion before differentiation into a differentiated cell type such as, for example, hepatocytes, myocytes, or adipocytes. Alternatively, in some instances, stem cells are differentiated into hepatocytes and subsequently transitioned to 3-dimensional suspension cultures. In some cases, differentiated cells are transdifferentiated into a desired cell type.

### Genetic Modification of Cell Lines

Disclosed herein are methods for performing one or more modifications on a cell or cell line. In some cases, the modification is a genetic modification carried out by introducing nucleic acids or genetic constructs into a cell or cell line. Cells can be modified to provide the capacity for self-renewal, differentiation into a desired cell type, obtaining a particular cell phenotype (e.g. steatosis), or other desirable changes. In some cases, cells are modified through introduction of exogenous nucleic acids such as one or more DNA construct(s). The introduction of foreign nucleic acids into the cell can be accomplished using various methods including, but not limited to, transfection, transduction, viral transduction, microinjection, lipofection, nucleofection, or transformation. For example, cells of a particular cell type can be transdifferentiated into a desired cell type.

In addition, gene editing systems such as TALENS (transcription activator-like effector nucleases) or CRISPR can be utilized to perform genetic modification in cells. For example, CRISPR can be customized because the active form consists of an invariant Cas9 protein and a programmable guide RNA (gRNA). The Cas9-gRNA complex probes DNA for the protospacer-adjacent motif (PAM) sequence followed by formation of an R-loop. Upon formation of a macromolecular complex comprising Cas9, gRNA, and the target DNA, the Cas9 protein generates two nicks in the target DNA, creating a blunt double-strand break that is predominantly repaired by the non-homologous end joining pathway or template-directed homologous recombination.

Genetic constructs can comprise a promoter and ORF(s) for one or more genes. A genetic construct may be introduced into a cell population followed by selection for a stable cell line that has incorporated the construct. For example, a plasmid comprising a gene of interest and a neo gene providing resistance to G418 can be linearized (e.g. cut once with a restriction endonuclease) and transfected into a duck hepatocyte fibroblast cell line and then selected with G418 to obtain fibroblasts successfully incorporating the linear plasmid vector into the genome. Examples of promoter include cytomegalovirus (CMV), CMV enhancer fused to chicken beta-actin promoter (CAG), human elongation factor 1-α (HEF-1α), telomerase reverse transcriptase (hTERT) promoter, and simian virus (SV40) promoter. In some cases, a promoter having a low or nonexistent basal transcription rate is used to minimize or prevent leaky expression. As an example, expression of a recombinase used in various constructs described herein can cause irreversible changes to a cell (e.g. by exciting genes involved in maintaining pluripotency). Thus, in some embodiments, the construct(s) comprises a promoter allowing no more than 1 transcription event per mitotic cell cycle. In some cases, the promoter allows no more than 1, 2, 3, 4, 5, 6, 7, 8, 9, 10, 20, 30, 40, or 50 transcription events per mitotic cell cycle (on average). In some cases, the promoter allows less than 1 transcription event per mitotic cycle (on average). Sometimes, the promoter allows no transcription events per mitotic cycle (e.g. when the average is less than half a transcription event per mitotic cycle).

The genetic modifications allow generation of cell lines that possess desired properties. For example, modified cell lines may express gene(s) that maintain the cell line in a state of self-renewal and/or proliferation. A state of self-renewal can be a state of proliferation or division while maintaining an undifferentiated state. As an example, induced pluripotent stem cells having the self-renewal property can be generated from differentiated adult cells by expressing one or more of Oct3/4, Sox2, Klf4 and c-Myc. In some cases, a cell can be both differentiated and have capacity for indefinite proliferation (e.g. immortalized fibroblasts). Sometimes, the modified cell lines are responsive to an inducing agent that triggers a switch from one phenotype to another. The switch can be from a state of self-renewal to a differentiated state (e.g. myosatellite cells into myocytes or pre-adipocytes into adipocytes). The methods disclosed herein can comprise one or more constructs for inducible adipogenesis. For example, the method may utilize a first construct comprising one or more pluripotency genes for promoting cell division and/or maintaining pre-adipocytes in an undifferentiated state, and a second construct comprising a TRE, one or more adipogenic genes, and a regulatory factor (e.g. Cre recombinase) for inactivating the pluripotency genes. In some cases, the switch comprises changing from one differentiated cell type into another differentiated cell type (e.g. from adult fibroblasts into hepatocytes). Sometimes, the switch does not entail a change in cell type but instead comprises a change in cell phenotype or a cellular characteristic. As an example, the switch can induce a hepatocyte to undergo steatosis, become predisposed to steatosis (e.g. more likely to undergo steatosis under the appropriate conditions such as incubation with a fatty acid), or results in enhanced steatosis (e.g. increased lipid accumulation compared to a control). Examples of genes implicated in adipogenesis include FABP4, GLUT4, ADIPOQ, AGPAT2, PLIN1, LEP, and LPL. In some instances, the construct comprises FABP4, GLUT4, ADIPOQ, AGPAT2, PLIN1, LEP, LPL, or any combination thereof. The construct can comprise at least one, at least two, at least three, at least four, at least five, at least six, or all seven of the ORFs for genes selected from the group consisting of FABP4, GLUT4, ADIPOQ, AGPAT2, PLIN1, LEP, and LPL.

In some instances, cells are modified to express (inducible expression or constitutively active expression) one or more pluripotency genes that promote cell division. In certain cases, the pluripotency gene(s) promote at least about 50, at least about 100, at least about 150, at least about 200, at least about 250, at least about 300, at least about 350, at least about 400, at least about 500, at least about 600, at least about 700, at least about 800, at least about 900, or at least about 1000 cell divisions. In some cases, the number of cell divisions for a given cell line or population of cells is monitored for quality control. For example, in some instances, cell lines or populations that exceed a threshold cell division count are not used for producing cultured food products. In some cases, the threshold cell division count is at least about 100, about 200, about 300, about 400, about 500, about 600, about 700, about 800, about 900, about 1000, about 2000, about 3000, about 4000, about 5000, about 6000, about 7000, about 8000, about 9000, about 10000, about 20000, about 30000, about 40000, or about 50000 or more cell divisions. In some cases, the threshold cell division count is no more than about 100, about 200, about 300, about 400, about 500, about 600, about 700, about 800, about 900, about 1000, about 2000, about 3000, about 4000, about 5000, about 6000, about 7000, about 8000, about 9000, about 10000, about 20000, about 30000, about 40000, or about 50000 or more cell divisions.

Disclosed herein are inducible cells that are modified using constructs that are responsive to an inducing agent. These modified cells can be used for generating cultured food products such as fish meat, avian liver tissue, and other foods. The modified cells can be used to control proliferation, differentiation, cell phenotype (e.g., steatosis/lipid accumulation), or other cellular properties. Exemplary non-limiting examples of such inducible systems are the Tet-on/off systems which utilize tetracycline/doxycycline as the inducing agent. Other inducible systems are also contemplated for carrying out the methods described herein. Examples of non-Tet inducible systems include the coumermycin inducible expression system, the RheoSwitch® Mammalian Inducible Expression system, estrogen receptor inducible systems, cumate-inducible systems, and Cre-Lox recombinase systems. In some cases, cell lines are generated that have stably incorporated the inducible systems or constructs described herein. Alternatively, cells can be modulated to transiently express the inducible systems or constructs described herein (e.g., via transient transfection of at least one construct).

A Tet-on or Tet-off system typically utilizes a tetracycline transactivator protein. TetO sequences are typically positioned upstream of any ORF(s) whose expression is sought to be controlled using the Tet system. A promoter and the TetO sequence(s) can make up a tetracycline response element (TRE). In some cases, the TRE consists of TetO sequence(s) and is placed upstream of a promoter and the ORF(s) for one or more genes of interest. In the Tet-on system, the transactivator protein has a strong binding affinity for TetO operator sequence(s) when it is not bound by tetracycline (or a derivative such as doxycycline). In the absence of tetracycline, the transactivator protein does not bind to the tetracycline response element (TRE). When tetracycline is added, it binds to the transactivator protein and causes the transactivator protein to bind to the TRE to induce expression of downstream ORF(s). In a Tet-off system, the transactivator protein has a strong binding affinity for TetO operator sequence(s) only when it is not bound by tetracycline. In the absence of tetracycline, the transactivator protein binds the TetO sequences and promotes expression of the downstream ORF(s). Added tetracycline binds to the transactivation protein causing a conformational change that results in decreased or loss of binding to the TRE, resulting in reduced expression of the downstream ORF(s).

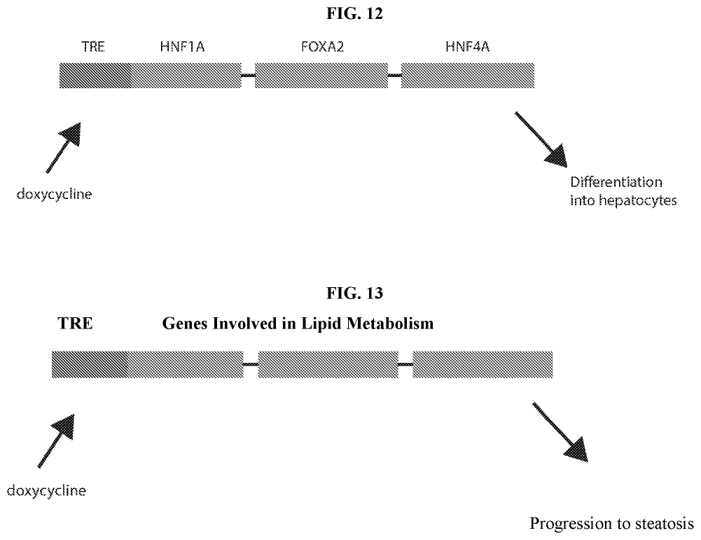

FIG. 12 shows an exemplary embodiment of a genetic construct that can be introduced into a cell to provide inducible differentiation into a hepatocyte. The construct comprises a tetracycline responsive element (TRE) and ORFs for the hepatocyte reprogramming factors HNF1A, FOXA1, and HNF4A. The construct can be stably transformed into a target cell such as a pluripotent or multipotent cell. In some cases, the construct can be stably transformed into a terminally differentiated cell such as an immortalized fibroblast (obtained according to the techniques described herein). Expression of the ORFs is normally suppressed in the absence of tetracycline. Treatment with tetracycline induces expression of the ORFs, which pushes the cells towards differentiation into a hepatocyte. Thus, a cell line stably incorporating this construct can be induced to differentiate into a hepatocyte via treatment with tetracycline/doxycycline. In certain cases, the construct comprises at least one of HNF1A, FOXA1, and HNF4A. Sometimes, the construct comprises at least two of HNF1A, FOXA1, and HNF4A. In some instances, the construct comprises HNF1A, FOXA1, and HNF4A. The construct can comprise HNF1A, FOXA1, HNF4A, or any combination thereof. In certain instances, the construct comprises HNF1A and FOXA1; HNF1A and HNF4A; or FOXA1 and HNF4A.

FIG. 13 shows an exemplary embodiment of a construct that can be introduced into a cell to allow inducible expression of one or more proteins that predispose the cell to steatosis. The construct comprises a tetracycline responsive element (TRE) and the ORF for one or more genes involved in lipid metabolism. The construct can be stably transformed into a target cell such as a pluripotent or multipotent cell. In some cases, the construct can be stably transformed into a terminally differentiated cell such as a fibroblast. The TRE suppresses expression of the ORFs but allows the ORFs to be transcribed in the presence of tetracycline or doxycycline. Thus, a cell line stably incorporating this construct can be induced to undergo steatosis or become predisposed to steatosis via treatment with tetracycline/doxycycline. In some cases, the construct comprises the ORF for ZFP423. In some cases, the construct comprises the ORF for ATF4 (activating transcription factor 3) (Kim J Y et al., Activating transcription factor 3 is a target molecule linking hepatic steatosis to impaired glucose homeostasis. J Hepatol. 2017 August; 67(2):349-359). In some cases, the construct comprises the ORF for SREBP-1c (Ferre P et al., Hepatic steatosis: a role for de novo lipogenesis and the transcription factor SREBP-1c. Diabetes Obes Metab. 2010 October; 12 Suppl 2:83-92). Other genes are contemplated for use in the methods and construct systems described herein and include: LPIN1, PPAR, APOC3, APOE, ORL1, PEMT, MTTP, SREBP, STAT3, KLF6, or any combination thereof. In some cases, the construct comprises at least one, at least two, at least three, at least four, at least five, at least six, at least seven, at least eight, at least nine, at least ten, or at least eleven genes selected from the group consisting of: ATF4, ZFP423, LPIN1, PPAR, APOC3, APOE, ORL1, PEMT, MTTP, SREBP, STAT3, and KLF6.

Exemplary genes utilized in the methods described herein are listed in Table 1 below. Expression of genes involved in lipid metabolism can be induced or enhanced to facilitate or enhance lipid accumulation and/or steatosis in target cells such as hepatocytes or adipocytes. Hepatocyte reprogramming factor(s) can be used to reprogram a cell type into hepatocytes such as transdifferentiation of fibroblasts into hepatocytes. Similarly, myocyte reprogramming factor(s) can be used to reprogram a cell type such as a fibroblast into a myocyte. Adipocyte reprogramming factor(s) can be used to reprogram a cell type such as a fibroblast into an adipocyte. Likewise, genes involved in myocyte differentiation and adipogenesis can be used to induce differentiation from myosatellite cells into myocytes and pre-adipocytes into adipocytes, respectively. Finally, various genes are listed that can be used to generate induced pluripotent stem cells (iPSCs). Any one gene or combination of genes listed in Table 1 is contemplated for the stated purposes described herein.

TABLE 1
Gene List
No./Gene Name/Gene Symbol and Synonyms/HGNC ID

Genes Involved in Lipid Metabolism/Steatosis
No. 1/Activating Transcription Factor 4/ATF4/786
No. 2/Lipin 1/LPIN1/13345
No. 3/Peroxisome Proliferator Activated Receptor Gamma/PPARG/9236
No. 4/Peroxisome Proliferator Activated Receptor Delta/PPARD/9235
No. 5/Peroxisome Proliferator Activated Receptor Alpha/PPARA/9232
No. 6/Apolipoprotein C3/APOC3/610
No. 7/Apolipoprotein E/APOE/613
No. 8/Opioid Related Nociceptin Receptor 1/ORL1/8155
No. 9/Phosphatidylethanolamine N-Methyltransferase/PEMT/8830
No. 10/Microsomal Triglyceride Transfer Protein/MTTP/7467
No. 11/Sterol Regulatory Element Binding Transcription Factor 1/SREBPF1/SREBP1/11289
No. 12/Sterol Regulatory Element Binding Transcription Factor 2/SREBPF2/SREBP2/11290
No. 13/Signal Transducer And Activator Of Transcription 3/STAT3/APRF/11364
No. 14/Kruppel Like Factor 6/KLF6/CPBP/ZF9/2235
No. 15/Zinc Finger Protein 423/ZFP423 /16762
Hepatocyte Reprogramming Factors
No. 16/Hepatocyte Nuclear Factor 1 Alpha/HNF1A/11621
No. 17/Forkhead Box A1/FOXA1/5021
No. 18/Hepatocyte Nuclear Factor 4 Alpha/HNF4A/5024
Myocyte Differentiation Factors
No. 19/Myogenin/MYOG/7612
No. 20/Myogenic Factor 6/MRF4/MYF6/7566
No. 21/Myogenic Factor 5/MYF5/7565
No. 22/Myogenic Differentiation 1/MYOD1/MYOD/7611
Genes Involved in Adipogenesis
No. 23/Fatty Acid Binding Protein 4/FABP4/3559
No. 24/Insulin-Responsive Glucose Transporter Type 4/GLUT4/SLC2A4/11009
No. 25/Adiponectin, C1Q And Collagen Domain Containing/ADIPOQ/13633
No. 26/1-Acylglycerol-3-Phosphate O-Acyltransferase 2/AGPAT2/325
No. 27/Perilipin 1/PLIN/PLIN1/9076
No. 28/Leptin/LEP/LEPD/6553
No. 29/Lipoprotein Lipase/LPL/LIPD/6677
Genes used to generate iPSCs
No. 30/Octamer-Binding Protein 3/4/OCT4/OCT3/9221
No. 31/SRY-Box 2/SOX2/11195
No. 32/Kruppel Like Factor 4/KLF4/6348
No. 33/MYC Proto-Oncogene, BHLH Transcription Factor/C-MYC/CMYC/7553
No. 34/MYCN Proto-Oncogene, BHLH Transcription Factor/N-MYC/NMYC/7559
No. 35/MYCL Proto-Oncogene, BHLH Transcription Factor/L-MYC/LMYC/7555
No. 36/Nanog Homeobox/NANOG/20857
No. 37/Lin-28 Homolog A/LIN28A/15986
No. 38/GLIS Family Zinc Finger 1/GLIS1/29525

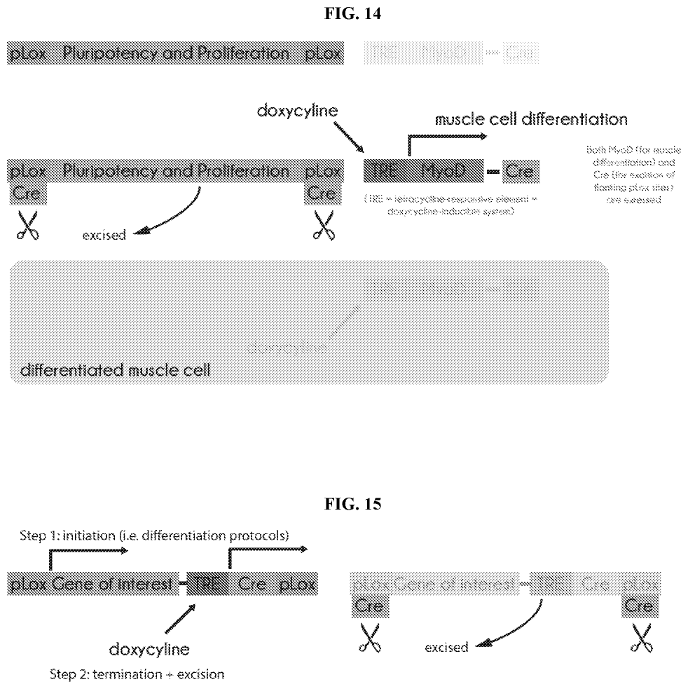

FIG. 14 shows an exemplary embodiment of a DNA construct system that can be introduced into a cell to allow a proliferation/differentiation switch from a pluripotent phenotype into a differentiated phenotype. This system has a first construct comprising a pluripotency cassette providing constitutive expression of the ORFs for pluripotency factors (e.g. Oct4, Sox2, Klf4, I-Myc). The pluripotency factors of the first construct are flanked by pLox sites. The system has a second construct comprising a differentiation cassette providing tetracycline inducible expression of MyoD and Cre recombinase. The addition of an inducing agent such as tetracycline or doxycycline can induce expression of MyoD and Cre recombinase. MyoD expression can help cause the cell to undergo differentiation into a muscle cell. The Cre recombinase enzyme can catalyze the excision of the pluripotency factors flanked by the pLox sites. Next, the inducing agent can be removed to cease induction of MyoD and Cre recombinase expression. An advantage of this system is the low footprint left by the system following excision of the pluripotency factors and removal of the inducing agent. Other genes can be used for inducing myogenesis and can include Myogenin (MyoG), MRF4, and Myf5. In some cases, MyoD, MyoG, MRF4, Myf5, or any combination thereof are used for inducing myogenesis. Sometimes, the methods and/or construct systems described herein utilize at least one, at least two, at least three, or all four myogenic factors selected from the group consisting of MyoD, MyoG, MRF4, and Myf5.

FIG. 15 shows an exemplary construct that can be introduced into a cell to provide an inducible “off-switch”. This construct comprises ORF(s) for one or more genes of interest and an expression cassette comprising TRE and Cre recombinase, which are flanked by pLox sites. The construct as shown in FIG. 15 comprises the TRE-Cre expression cassette located downstream of the ORF(s). Alternatively, in construct can have the TRE-Cre expression cassette located upstream of the ORF(s). A promoter is generally positioned upstream of the ORF(s) and is a separate promoter from the TRE such that the ORF(s) are expressed independently of the Cre recombinase. Addition of an inducing agent can cause a cell line stably incorporating this construct to express Cre recombinase for catalyzing the excision of the intervening sequence flanked by the pLox sites. Thus, the one or more genes (e.g., one(s) that promote differentiation) and the TRE and Cre recombinase expression cassette are removed, resulting in footprint-free excision of the genes of interest. Such constructs can be used for inducing transdifferentiation of cells into a different cell type.

FIG. 28A shows one embodiment of a synthetic receptor for modulating gene expression (e.g., activating and/or inactivating target ORFs). This system is modeled after the “synthetic notch receptor” system described in Morsut et al., Engineering Customized Cell Sensing and Response Behaviors Using Synthetic Notch Receptors. Cell. 2016 Feb. 11; 164(4):780-91. In this system, a receptor is engineered to comprise an extracellular component that is the same as endogenous Notch receptors (which signal by cleaving intracellular domains upon binding a ligand, such as the protein Delta). Accordingly, the intracellular domain can be replaced by an enzyme, such as Cre recombinase. When a ligand is added to the cell, the engineered Notch receptor becomes activated, and the intracellular domain is cleaved, which in this case releases Cre into the cytoplasm. Cre enters the nucleus by passive diffusion (or has a nuclear localization sequence engineered into the protein, facilitating entry into the nucleus). There, it induces recombination of loxP sites, as described herein (e.g., FIGS. 12-15). Such constructs can be used for inducing transdifferentiation of cells into a different cell type.

The model shown in FIG. 28A represents an irreversible switch, by which Cre is released from the cell membrane and into the nucleus to induce recombination events. Such events (including the switch from maintenance of pluripotency to differentiation) are described herein. Cre delivery has the potential to affect various cellular functions or properties, such as proliferation, adhesion, differentiation, migration, and other cell properties. In various embodiments, this switch allows the transition from a proliferative state to the activation of genes that induce differentiation (into myocytes, adipocytes, etc). An important benefit of this system is that it does not require activation of genes to enable Cre to function; instead, the enzyme is constitutively expressed and localized as reservoirs at the cell surface. Such constructs can be used for inducing transdifferentiation of cells into a different cell type.

FIG. 28B shows additional strategies for switching between gene sets (e.g., inactivating a pluripotency gene set and activating a differentiation gene set). These strategies can implement site-specific recombinase (SSR) systems in combination with inducible gene expression systems (e.g., tetracycline/doxycycline inducible systems). The SSR/inducible combination is described in Zhang et al. Conditional gene manipulation: Cre-ating a new biological era. J Zhejiang Univ-Sci B (Biomed & Biotechnol) 2012 13(7):511-524. In some cases, this SSR/inducible system is used as a switch between pluripotency and differentiation for creating ex vivo meat. Such constructs can be used for inducing transdifferentiation of cells into a different cell type.

As shown in FIG. 28B, the triangles represent lox sequences (black is loxP and white is lox5171, although other sequences may be used for this purpose). When the triangles are aligned, a recombination event (catalyzed by the Cre enzyme) leads to excision/deletion of the sequence between them. When the triangles (representing defined DNA sequences) are facing each other, a recombination event leads to stochastic inversion (flipping back and forth) of the intervening sequence. For the purpose of this illustration, proliferation is described generally (and can encompass genes such as members of the cyclin family, cyclin-dependent kinases, cell cycle inhibitors such as p27kip, TERT, and others). Proliferation can also be substituted with “Pluripotency,” genes (such as the Yamanaka factors for generating iPSCs), as detailed in other gene switch mechanisms described herein (e.g., FIGS. 12-15). Likewise, differentiation can encompass various genes (such as MyoD in the case of myocytes). Other genes may be used in the same way, stimulating differentiation to any lineage. Such constructs can be used for inducing transdifferentiation of cells into a different cell type.

Two representative scenarios are shown in FIG. 28B. In the upper panel (1), when Cre is added, it first induces recombination in either the black pair or white pair of triangles. When the initial event involves the black triangles (loxP) as shown in the left scenario, this induces inversion of the intervening DNA sequence, which puts two white triangles (lox5171) in parallel; this permits the Cre enzyme to then excise the intervening sequence. The result is a switch from proliferation genes to differentiation genes. In the right scenario, Cre first acts upon the white triangles (lox5171). This induces an inversion that then places the black triangles (loxP) in parallel, permitting Cre to then excise the intervening sequence. Again, the result is a switch from transcription of proliferation genes to transcription of differentiation genes. Such constructs can be used for inducing transdifferentiation of cells into a different cell type.

In the lower panel (2), a different schematic yields a switch from proliferation to differentiation. The lox sequences are placed such that when Cre first induces recombination between the white triangles (lox5171), an inversion event occurs. The inversion event puts the two black triangles in parallel (loxP), enabling Cre to excise the proliferation genes and activate differentiation. The inverse occurs in the right panel in which Cre first induces recombination between the black triangles. Such constructs can be used for inducing transdifferentiation of cells into a different cell type.

Unlike other systems (which require the activation of one gene program, followed by a second step of gene activation), the processes described herein in FIG. 28B are unique in that they induce a complete switch from one gene set to another, using a single input (Cre), with very high efficiency. This approach provides an improvement in the method of generating cultured food products that can simplify the process and/or reduce required inputs, especially at large-scale production. At scale, the processes disclosed herein represent a significant improvement in process simplification, and a reduction in requisite inputs.

The inducible systems described herein are not limited to Tet and/or Cre recombinase based systems. Other embodiments of a system utilizing inducible recombinase expression to excise one or more genes of interest are contemplated. For example, the Flp-FRT system utilizes Flp (flippase) recombinase to excise DNA flanked by FRT (flippase recognition target) sequences. Such systems can be used for inducing transdifferentiation of cells into a different cell type.

The combination of induced expression and gene excision to maintain cells in an undifferentiated state of self-renewal may face a technical problem of promoter leakiness or a basal expression level. For example, leakiness in the promoter could result in some expression of the recombinase, which then excises the pluripotent stem cell factors before the inducing agent is added. However, a key advantage of utilizing this system for purposes of cultured food production as described herein is that those cells that experience leakiness will likely lose their self-renewal phenotype and become outcompeted by the cells that maintain tighter control of recombinase expression. Thus, when self-renewing (e.g. pluripotent or multipotent) cell cultures are scaled up to produce commercial quantities of cultured food products, most or all of the cultured cells in the population should retain their undifferentiated and self-renewing properties when the basal expression level is sufficiently low. Modified cells may be clonally selected to identify cell lines that have strong repression of recombinase expression in the absence of an inducing agent. In some cases, the inducing agent is added to induce differentiation (and/or other desired properties) shortly before the cells are harvested to produce the meat product for human consumption.

### Inducing Lipid Accumulation or Steatosis

In some cases, the systems, methods, and compositions disclosed herein provide for inducing lipid accumulation or steatosis. Oftentimes, lipid accumulation or steatosis is induced in a population of cells for purposes of producing a cell cultured food product with increased lipid content. As used herein, steatosis is a pathologic state characterized by the abnormal retention of lipids within a cell. The excess lipids accumulate in vesicles that displace the cytoplasm. Macrovesicular steatosis describes when the vesicles are large enough to displace or distort the nucleus, while microvesicular steatosis lacks this phenotype. For example, FIG. 4A illustrates a process including the generation of steatotic hepatocytes 413 by genetic intervention and/or addition of exogenous compounds 409.

In some aspects, lipid accumulation and/or steatosis is induced in a population of cells by genetic manipulation. For example, some methods disclosed herein provide for the preparation of foie gras comprising cultured avian liver tissue. Some such methods comprise: a) obtaining a population of avian derived cells capable of self-renewal; b) differentiating the population of avian derived cells into hepatocytes; and c) inducing steatosis in the hepatocytes to generate cultured avian liver tissue having high lipid content; and d) preparing the cultured avian liver tissue as foie gras. In certain cases, non-hepatocytes are induced to undergo lipid accumulation. Some methods produce cultured cells having high lipid accumulation for human consumption. One example of such a method comprises: a) culturing a population of cells; b) inducing differentiation within the population of cells; c) inducing high lipid accumulation within the population of cells; and d) processing the population of cells for human consumption.

Steatosis and/or lipid accumulation is accomplished by manipulating lipid metabolism. For example, the genetic profiles of hepatocytes that have undergone steatosis has been previously characterized by Chiappini et al., Exploration of global gene expression in human liver steatosis by high-density oligonucleotide microarray. Lab Invest. 2006 February; 86(2):154-65. Affected intracellular signaling pathways in steatotic hepatocytes involve lipid metabolism, and lead to the accumulation of lipid droplets within the cytoplasm of hepatocytes. In some cases, the systems and methods described herein provide for reliable, high-efficiency induction of steatosis in a population of cells. Steatosis is often induced in a population of liver cells or hepatocytes. Sometimes, steatosis is induced by up or downregulation of genes involved in hepatic lipid metabolism. For example, in some cases, p53 depletion and/or upregulation of p63 (e.g. overexpression of N-terminal transactivation domain TAp63) induces lipid accumulation as described in Porteiro et al., Hepatic p63 regulates steatosis via IKK beta/ER stress. Nature Communications. 2017 May; 8:15111. Genetic modification of cells such as hepatocytes may be carried out using the various protocols discussed (e.g. inducible expression of genes involved in steatosis). In some instances, steatosis and/or lipid accumulation is induced by manipulating at least one of lipid metabolism pathway(s) and ER pathway(s) involved in ER stress. Sometimes, steatosis or lipid accumulation is induced in hepatocytes or liver cells. Alternatively, in other cases, steatosis or lipid accumulation is induced in non-hepatocyte cells such as, for example, myocytes or skeletal muscle cells. Sometimes, steatosis is induced in fish myocytes such as salmon myocytes. In certain instances, steatosis or lipid accumulation is induced in non-hepatocyte organ cells such as, for example, kidney cells. On occasion, steatosis or lipid accumulation is induced in a pluripotent cell population or an adult progenitor cell population that is used as an intermediate cell line for expansion into a differentiated cell population. In these scenarios, steatosis or lipid accumulation is induced earlier during the production process before the population of cells is differentiated. In some cases, high lipid accumulation is induced in cells.

Conversely, in certain instances, steatosis is induced by disrupting lipid metabolism pathways without requiring genetic manipulation. For example, certain exogenous compounds are capable of inducing steatosis in hepatocytes grown in vitro or ex vivo. These exogenous compounds include toxins such as alcohols, and lipids such as fatty acids. In some cases, culturing cells in a media formulation having a high concentration of at least one lipid induces steatosis. Cells are cultured in a lipid rich media formulation to induce steatosis, in various embodiments. The cells cultured in the lipid rich media sometimes comprise a population of differentiated cells such as, for example, avian hepatocytes or fish myocytes. In certain cases, the cells cultured in the lipid rich media are pluripotent stem cells such as embryonic stem cells or induced pluripotent stem cells. Alternatively, the cells cultured in the lipid rich media are multipotent stem cells such as adult progenitor cells, in certain instances. Sometimes, cells are cultured in a lipid rich media with at least one lipid type selected from saturated fatty acids, mono-unsaturated fatty acids, polyunsaturated fatty acids, and trans-fatty acids. Examples of lipids include palmitic acid, oleic acid, docosahexaenoic acid, stearic acid, linoleic acid, linolenic acid, arachidonic acid, and eicosapentaenoic acid. In some cases, the culture media is supplemented with linoleic acid, oleic acid, or a combination thereof to induce lipid accumulation or steatosis within a population of cells cultured in the media. In some cases, the culture media is supplemented with a lipid at a concentration of at least about 1 mM, about 2 mM, about 3 mM, about 4 mM, about 5 mM, about 6 mM, about 7 mM, about 8 mM, about 9 mM, about 10 mM, about 15 mM, about or 20 mM. Sometimes, the culture media is supplemented with a lipid at a concentration of no more than about 1 mM, about 2 mM, about 3 mM, about 4 mM, about 5 mM, about 6 mM, about 7 mM, about 8 mM, about 9 mM, about 10 mM, about 15 mM, or about 20 mM. In various embodiments, the culture media is supplemented with a lipid at a concentration of at least about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 200 μM, about 300 μM, about 400 μM, about 500 μM, about 600 μM, about 700 μM, about 800 μM, about 900 μM, or about 1000 μM. In certain instances, the culture media is supplemented with a lipid at a concentration of no more than about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 200 μM, about 300 μM, about 400 μM, about 500 μM, about 600 μM, about 700 μM, about 800 μM, about 900 μM, or about 1000 μM. Oftentimes, the cell culture media comprises a lipid concentration of about 1 mM to about 20 mM or of about 1 μM to about 1000 μM. The cell culture media often comprises a lipid concentration of at least about 1 μM. Typically, the cell culture media comprises a lipid concentration of at most about 20 mM.

In some cases, the culture media is supplemented with at least one culture media supplement such as IBMX (a methyl xanthine), rosiglitazone (a thiazolidinedione), increased glucose concentration, and/or a corticosteroid such as dexamethasone. Other examples of thiazolidinediones that can be used to supplement culture media include pioglitazone, lobeglitazone, ciglitazone, darglitazone, englitazone, netoglitazone, rivoglitazone, troglitazone, and balaglitazone. In certain instances, the culture media is supplemented with at least one thiazolidinedione selected from the group consisting of pioglitazone, lobeglitazone, ciglitazone, darglitazone, englitazone, netoglitazone, rivoglitazone, troglitazone, and balaglitazone. In one example, the thiazolidinedione is rosiglitazone. The culture media supplements described herein can be added to the culture media at various concentrations including the full range of concentrations described for lipid concentrations. For example, a culture media supplement can be added to the media at a concentration of at least about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 200 μM, about 300 μM, about 400 μM, about 500 μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 15 mM, or about 20 mM. In some cases, a culture media supplement can be added to the media at a concentration of no more than about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 200 μM, about 300 μM, about 400 μM, about 500 μM, about 600 μM, about 700 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 15 mM, or about 20 mM.

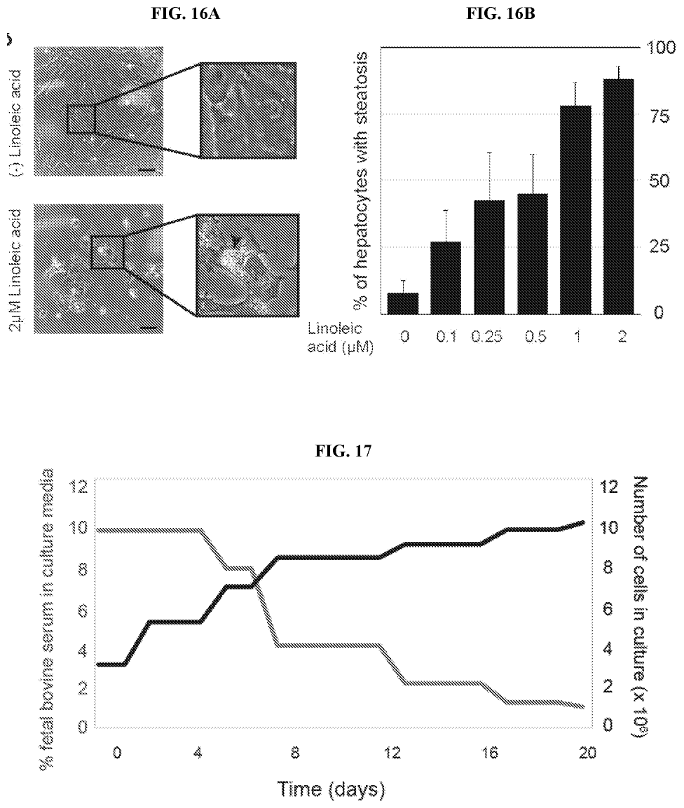

FIG. 16A shows successful induction of steatosis (accumulation of intracellular lipid-containing vesicles; arrowhead) in duck hepatocytes upon incubation with 2 μM linoleic acid (bottom panel) compared to the untreated control (top panel). FIG. 16B shows a dose response curve correlating the percentage of steatotic hepatocytes with the concentration of linoleic acid. Similar results have been achieved with oleic acid. In some cases, the protocols were augmented by incubating the hepatocytes with IBMX (a methyl xanthine), rosiglitazone (a thiazolidinedione), increased glucose concentration, or other fatty acid species, and corticosteroids such as dexamethasone.

In some cases, the cell culture media comprises a lipid concentration (or other media supplement described herein) of at least about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 3 μM, about 4 μM, about 5 μM, about 6 μM, about 7 μM, about 8 μM, about 9 μM, about 10 μM, about 15 μM, or about 20 μM. In some cases, the cell culture media comprises a lipid concentration of at most about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 3 μM, about 4 μM, about 5 μM, about 6 μM, about 7 μM, about 8 μM, about 9 μM, about 10 μM, about 15 μM, or about 20 μM. In some cases, the cell culture media comprises a lipid concentration of about 1 μM to about 2 μM, about 1 μM to about 3 μM, about 1 μM to about 4 μM, about 1 μM to about 5 μM, about 1 μM to about 6 μM, about 1 μM to about 7 μM, about 1 μM to about 8 μM, about 1 μM to about 9 μM, about 1 μM to about 10 μM, about 1 μM to about 15 μM, about 1 μM to about 20 μM, about 2 μM to about 3 μM, about 2 μM to about 4 μM, about 2 μM to about 5 μM, about 2 μM to about 6 μM, about 2 μM to about 7 μM, about 2 μM to about 8 μM, about 2 μM to about 9 μM, about 2 μM to about 10 μM, about 2 μM to about 15 μM, about 2 μM to about 20 μM, about 3 μM to about 4 μM, about 3 μM to about 5 μM, about 3 μM to about 6 μM, about 3 μM to about 7 μM, about 3 μM to about 8 μM, about 3 μM to about 9 μM, about 3 μM to about 10 μM, about 3 μM to about 15 μM, about 3 μM to about 20 μM, about 4 μM to about 5 μM, about 4 μM to about 6 μM, about 4 μM to about 7 μM, about 4 μM to about 8 μM, about 4 μM to about 9 μM, about 4 μM to about 10 μM, about 4 μM to about 15 μM, about 4 μM to about 20 μM, about 5 μM to about 6 μM, about 5 μM to about 7 μM, about 5 μM to about 8 μM, about 5 μM to about 9 μM, about 5 μM to about 10 μM, about 5 μM to about 15 μM, about 5 μM to about 20 μM, about 6 μM to about 7 μM, about 6 to about 8 μM, about 6 μM to about 9 μM, about 6 μM to about 10 μM, about 6 μM to about 15 μM, about 6 μM to about 20 μM, about 7 μM to about 8 μM, about 7 μM to about 9 μM, about 7 μM to about 10 μM, about 7 μM to about 15 μM, about 7 μM to about 20 μM, about 8 μM to about about 8 μM to about 10 μM, about 8 μM to about 15 μM, about 8 μM to about 20 μM, about 9 μM to about 10 μM, about 9 μM to about 15 μM, about 9 μM to about 20 μM, about 10 μM to about 15 μM, about 10 μM to about 20 μM, or about 15 μM to about 20 μM.

In some cases, the cell culture media comprises a lipid concentration (or other media supplement described herein) of at least about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, about 2 mM, about 3 mM, about 4 mM, about 5 mM, about 6 mM, about 7 mM, about 8 mM, about 9 mM, about 10 mM, about 15 mM, or about 20 mM. In some cases, the cell culture media comprises a lipid concentration of at most about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, about 2 mM, about 3 mM, about 4 mM, about 5 mM, about 6 mM, about 7 mM, about 8 mM, about 9 mM, about 10 mM, about 15 mM, or about 20 mM. In some cases, the cell culture media comprises a lipid concentration of about 1 mM to about 2 mM, about 1 mM to about 3 mM, about 1 mM to about 4 mM, about 1 mM to about 5 mM, about 1 mM to about 6 mM, about 1 mM to about 7 mM, about 1 mM to about 8 mM, about 1 mM to about 9 mM, about 1 mM to about 10 mM, about 1 mM to about 15 mM, about 1 mM to about 20 mM, about 2 mM to about 3 mM, about 2 mM to about 4 mM, about 2 mM to about 5 mM, about 2 mM to about 6 mM, about 2 mM to about 7 mM, about 2 mM to about 8 mM, about 2 mM to about 9 mM, about 2 mM to about 10 mM, about 2 mM to about 15 mM, about 2 mM to about 20 mM, about 3 mM to about 4 mM, about 3 mM to about 5 mM, about 3 mM to about 6 mM, about 3 mM to about 7 mM, about 3 mM to about 8 mM, about 3 mM to about 9 mM, about 3 mM to about 10 mM, about 3 mM to about 15 mM, about 3 mM to about 20 mM, about 4 mM to about 5 mM, about 4 mM to about 6 mM, about 4 mM to about 7 mM, about 4 mM to about 8 mM, about 4 mM to about 9 mM, about 4 mM to about 10 mM, about 4 mM to about 15 mM, about 4 mM to about 20 mM, about 5 mM to about 6 mM, about 5 mM to about 7 mM, about 5 mM to about 8 mM, about 5 mM to about 9 mM, about 5 mM to about 10 mM, about 5 mM to about 15 mM, about 5 mM to about 20 mM, about 6 mM to about 7 mM, about 6 mM to about 8 mM, about 6 mM to about 9 mM, about 6 mM to about 10 mM, about 6 mM to about 15 mM, about 6 mM to about 20 mM, about 7 mM to about 8 mM, about 7 mM to about 9 mM, about 7 mM to about 10 mM, about 7 mM to about 15 mM, about 7 mM to about 20 mM, about 8 mM to about 9 mM, about 8 mM to about 10 mM, about 8 mM to about 15 mM, about 8 mM to about 20 mM, about 9 mM to about 10 mM, about 9 mM to about 15 mM, about 9 mM to about 20 mM, about 10 mM to about 15 mM, about 10 mM to about 20 mM, or about 15 mM to about 20 mM.

In some cases, cells are cultured in a high lipid concentration for a period of time to induce steatosis. The length of time during which the cells are exposed to high lipid concentrations will vary depending on the cell type, size of the cell population, age of the cell population, number of passages, any genetic modification or manipulation of the cells, the type and components of the culture media, desired amount of lipid accumulation or steatosis, or any combination thereof. For example, certain cell types will intake exogenous lipids in the culture media at a slower rate than other cell types, and thus require a longer incubation period in a lipid rich media to induce the desired amount of steatosis. In various cases, cells are cultured in a cell culture media comprising at least one lipid for a period of time. Sometimes, cells are cultured in a culture media having a high lipid concentration for at least a certain period of time. In many cases, cells are cultured in a culture media having a high lipid concentration for about 1 day to about 20 days. Cells are often cultured in a culture media having a high lipid concentration for at least about 1 day. Typically, cells are cultured in a culture media having a high lipid concentration for at most about 20 days.

In certain instances, cells are cultured in a culture media having a high lipid (or other media supplement described herein) concentration for about 1 day to about 2 days, about 1 day to about 3 days, about 1 day to about 4 days, about 1 day to about 5 days, about 1 day to about 6 days, about 1 day to about 7 days, about 1 day to about 8 days, about 1 day to about 9 days, about 1 day to about 10 days, about 1 day to about 15 days, about 1 day to about 20 days, about 2 days to about 3 days, about 2 days to about 4 days, about 2 days to about 5 days, about 2 days to about 6 days, about 2 days to about 7 days, about 2 days to about 8 days, about 2 days to about 9 days, about 2 days to about 10 days, about 2 days to about 15 days, about 2 days to about 20 days, about 3 days to about 4 days, about 3 days to about 5 days, about 3 days to about 6 days, about 3 days to about 7 days, about 3 days to about 8 days, about 3 days to about 9 days, about 3 days to about 10 days, about 3 days to about 15 days, about 3 days to about 20 days, about 4 days to about 5 days, about 4 days to about 6 days, about 4 days to about 7 days, about 4 days to about 8 days, about 4 days to about 9 days, about 4 days to about 10 days, about 4 days to about 15 days, about 4 days to about 20 days, about 5 days to about 6 days, about 5 days to about 7 days, about 5 days to about 8 days, about 5 days to about 9 days, about 5 days to about 10 days, about 5 days to about 15 days, about 5 days to about 20 days, about 6 days to about 7 days, about 6 days to about 8 days, about 6 days to about 9 days, about 6 days to about 10 days, about 6 days to about 15 days, about 6 days to about 20 days, about 7 days to about 8 days, about 7 days to about 9 days, about 7 days to about 10 days, about 7 days to about 15 days, about 7 days to about 20 days, about 8 days to about 9 days, about 8 days to about 10 days, about 8 days to about 15 days, about 8 days to about 20 days, about 9 days to about 10 days, about 9 days to about 15 days, about 9 days to about 20 days, about 10 days to about 15 days, about 10 days to about 20 days, or about 15 days to about 20 days.

### Media Formulations

Provided herein are systems and methods utilizing at least one media formulation that enables cultured food production. In some cases, the media formulation does not require the use of serum such as fetal bovine serum. Sometimes, the media formulation does not require one or more other supplements that are used in certain cell culture media. Cell culture media is generally divided into two categories: serum media and serum-free media. Conventional media formulations often utilize fetal bovine serum and other supplements that are cost prohibitive for large scale cultured food production. Serum (e.g. fetal bovine serum) tends to vary between batches since it is produced from animals. For example, fetal bovine serum (FBS) is extracted from the blood of calf fetuses and tends to have batch-to-batch variation in composition. In addition, the use of serum can create the possibility of contamination by viruses, mycoplasma, prions, toxins, and other undesirables present in the animal from which the serum is extracted. Finally, serum is cost prohibitive and requires the raising of livestock, which is contrary to some of the goals of providing cultured food products. The use of serum-free media, however, bypasses these challenges. Substitutes or supplements to serum are used in various media formulations for producing the cultured food products described herein. Serum substitutes or supplements are derived from non-livestock sources (e.g. not derived from cow fetuses). Examples include mammalian cell overexpression systems and transgene expression in yeast, larger fungi (e.g. mushrooms), bacteria, algae, or insect cell (e.g. baculovirus) systems. An exemplary embodiment is a mushroom-based system for producing a serum substitute for serum-free media formulation(s) such as described in Benjaminson et al. In vitro edible muscle protein production system (mpps): Stage 1, fish. Acta Astronautica (2002): 51(12), 879-889. Sometimes, the systems, methods, and compositions described herein comprise generating or obtaining at least one cell line suitable for being cultured using a mushroom-based culture media formulation. In some cases, a hepatocyte cell line, a pre-adipocyte cell line, or a satellite cell line is conditioned or modified to enable culturing in a mushroom-based serum-free media formulation.

In some cases, a media formulation comprises a natural media. Oftentimes, a media formulation comprises a synthetic media or a modification thereof. Examples of synthetic media include Minimum Essential Media (MEM), Essential 8 Media, Basal Medium Eagle (BME), Ham's F12, Ham's F-10, Fischer's Medium, CMRL-1066 Medium, Click's Medium, Medium 199, Dulbecco's Modified Eagle's Media (DMEM), RPMI-1640, L-15 medium, McCoy's 5A Modified Medium, William's Medium E, and Iscove's Modified Dulbecco's Medium (IMDM).

In some cases, the media formulation is modified for culturing embryonic stem cells, induced pluripotent stem cells, embryonic germ cells, differentiated cells (e.g. hepatocytes or myocytes), immortalized differentiated cells, or nascent hepatic stem cells. In certain instances, a media formulation for culturing isolated duck stem cell lines as described in WO2008129058A1 is used with one or more modifications. For example, interleukin-6 and Stem Cell Factor are optionally eliminated from the media formulation, in some cases. Sometimes, the media formulation is modified from WO2008129058A1. The media formulation usually enables the proliferation and/or maintenance of self-renewal ability of stem cells without requiring feeder cells. For example, Essential 8 Medium provides the most important components for maintaining pluripotent stem cells in a feeder cell-free environment. A feeder-free culture environment enhances the large-scale production of cultured food products because it avoids having to constantly re-seed feeder cell layers in order to grow pluripotent stem cells. Oftentimes, multiple media formulations are used during the culturing of a population of cells. In some cases, an initial population of cells with self-renewal ability is cultured with a media formulation that maintains the self-renewal ability such as, for example, maintaining a population of embryonic stem cells in an un-differentiated state. Next, sometimes, differentiation is induced in the population of cells having self-renewal ability. As an example, the embryonic stem cells are induced to differentiate into hepatocytes. This differentiation step sometimes requires a differentiation media formulation. For example, in some instances, certain differentiation factor(s) are added and/or factor(s) required for maintaining the self-renewal ability are removed in the differentiation media. Moreover, when differentiation in the population of cells leads to the generation of hepatocytes, there is often an additional step of inducing steatosis or lipid accumulation in the hepatocytes. In some cases, steatosis is induced at least in part by the use of a steatotic media formulation. For example, a steatotic media formulation sometimes comprises a lipid concentration of at least a certain concentration as described elsewhere herein.

In some cases, a media formulation comprises at least one nutrient or nutritional supplement for enhancing the nutritional content of the finished food product. A nutrient is a macronutrient or a micronutrient. Macronutrients are nutrients that are needed in large quantities and include proteins, fats, and carbohydrates. Micro-nutrients are required in small quantities and include vitamins, minerals, some amino acids, and certain compounds such as, for example, flavonoids. In certain instances, at least one nutrient is added to a media formulation for intake by a population of cultured cells. As an example, steatotic hepatocytes used to produce foie gras typically have high lipid accumulation inside of the cytoplasm. Culturing the hepatocytes in a media formulation having a specific lipid composition (e.g. omega-3 fatty acids) may induce the resulting steatotic hepatocytes to have a modified lipid profile partially reflecting the lipid composition of the culture media. Certain methods provide for production of cultured tissue having increased nutritional content for human consumption. For example, some such methods comprise: a) culturing a population of cells in a culture medium having at least one nutritional supplement; b) manipulating lipid metabolic pathways to induce steatosis in the population of differentiated cells such that the cells accumulate high lipid content; and c) processing the population of differentiated cells into non-textured tissue for human consumption. Other methods comprise: a) culturing a population of cells in a culture medium having at least one nutritional supplement; b) manipulating lipid metabolic pathways to induce steatosis in the population of differentiated cells such that the cells accumulate high lipid content; and c) processing the population of differentiated cells into homogeneously textured tissue for human consumption.

In some cases, media formulations are produced that have certain growth factors, proteins, lipids, hormones, or any combination thereof required for cell culturing. Oftentimes, mammalian cell overexpression systems are used to generate any of the foregoing media components. In some instances, transgene expression in yeast, certain fungi, bacteria, algae, or insect cell (baculovirus) systems are utilized. The expressed media components are then isolated and/or purified in certain cases. Sometimes, the media formulations are generated using media conditioning technique(s). Alternatively, cells are sometimes cultured using co-culture models. However, in various cases, cells are cultured without co-culturing, or without co-culturing with xenobiotic cells such as yeast (e.g. organisms or cells that do not belong in the same kingdom, phylum, and/or species as the cells being cultured to produce food). For example, some avian cells are co-cultured with non-yeast cells such as mouse feeder cells.

Successful reduction or elimination of fetal bovine serum from cell culture media has been demonstrated. FIG. 17 shows a graph plotting the number of cells from an immortalized cell line derived from adult duck hepatocytes. These immortalized cells were cultured in progressively decreasing concentrations of fetal bovine serum (FBS) in the presence of soybean hydrolysate (10 g/L). The media supplementation of soybean hydrolysate allowed the serum requirements of the cultured hepatocytes to be reduced by 92%.

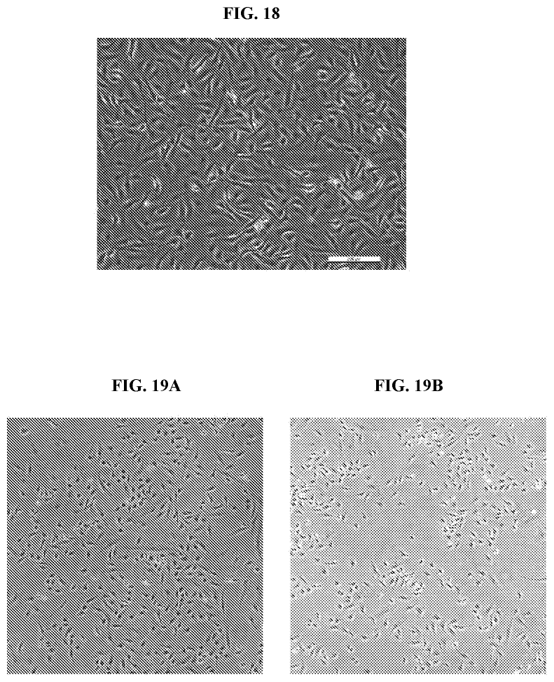

FIG. 18 shows duck fibroblasts that have also been successfully grown in 10% shiitake mushroom extract after successive reduction of fetal bovine serum from the cell culture media. In some cases, the culture media is supplemented with at least 1%, 2%, 3%, 4%, 5%, 6%, 7%, 8%, 9%, 10%, 11%, 12%, 13%, 14%, or 15% mushroom extract. In certain instances, the culture media is supplemented with no more than 1%, 2%, 3%, 4%, 5%, 6%, 7%, 8%, 9%, 10%, 11%, 12%, 13%, 14%, or 15% mushroom extract. The culture media supplemented with mushroom extract can utilize a reduced serum concentration or no serum. In some cases, the supplemented culture media has no more than 1%, 2%, 3%, 4%, 5%, 6%, 7%, 8%, 9%, or 10% serum (e.g., animal serum such as FBS). Such reduced serum or no-serum culture media formulations can be utilized for growing or culturing any of the cells described herein including cells derived from various species such as avian cells, fish cells, porcine cells, bovine cells, and the cells of other edible species. In some cases, the cells are derived from domesticated species (e.g., cows, pigs, chickens, ducks, etc). In other cases, the cells are derived from non-domesticated species (trout, salmon, lobsters, crabs, etc). Various cell types can be cultured in the reduced serum or no-serum culture media formulations described herein, including multipotent cells, pluripotent cells, embryonic stem cells, induced pluripotent stem cells, myocytes, adipocytes, myosatellite cells, pre-adipocytes, mesenchymal stem cells, fibroblasts, hepatocytes, and other cell types.

In some cases, a population of cells or a cell line is adapted to grow in reduced serum or no-serum culture media formulations without requiring supplementation. FIG. 19A shows duck fibroblasts grown in serum-free media without additional supplementation; FIG. 19B shows a control culture grown in DMEM supplemented with 10% fetal bovine serum.

### Scalable Production of Cultured Cells

Various methods are optionally used to scale up production of cultured cells for making food products for human consumption. The systems and methods disclosed herein enable the large-scale production of cultured foods (FIGS. 4A-4B). One method is to use 2-Dimensional surfaces such as tissue culture dishes or their functional equivalents (e.g. a cell culture chamber). A typical example would be a cell culture chamber having a polystyrene surface treated to increase hydrophilicity for enhancing attachment of adherent cells. Sometimes, the cell culture chamber is coated with a protein composition that acts as a substrate for cultured cells. The cell culture chamber often uses a media formulation as described herein. In many cases, the 2D surface approach is scaled up by combining a plurality of cell culture chambers. Sometimes, the plurality of cell culture chambers are stacked and arranged side by side. In some instances, cell culture chambers are stacked at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 chambers high. The stacks of cell culture chambers are usually arranged alongside each other. For example, in certain instances, the stacks of cell culture chambers are arranged side by side and/or front to back to maximize the usage of space. In many cases, the chambers are physically coupled (e.g. welded together as a single unit) and have common fill and vent ports connected by channels that allow liquid and air flow.

In certain embodiments, provided herein are bioreactor systems for culturing cells. Certain bioreactor systems facilitate production of cultured tissues suitable for human consumption. For example, some bioreactor systems comprise: a) a reactor chamber comprising a plurality of micro-scaffolds that provide adhesion surfaces for cellular attachment; b) a population of self-renewing cells cultivated within bioreactor; c) a first source providing at least one maintenance media comprising components for maintaining the population of self-renewing cells without spontaneous differentiation; and d) a second source providing at least one differentiation media comprising components for differentiating the population of self-renewing cells into a specific lineage; wherein the reactor chamber receives maintenance media from the first source to cultivate the population of cells and receives differentiation media from the second source to differentiate the population of cells, wherein the population of cells generated in a single batch comprises cultured tissues suitable for human consumption and having a dry weight of at least 1 kg.

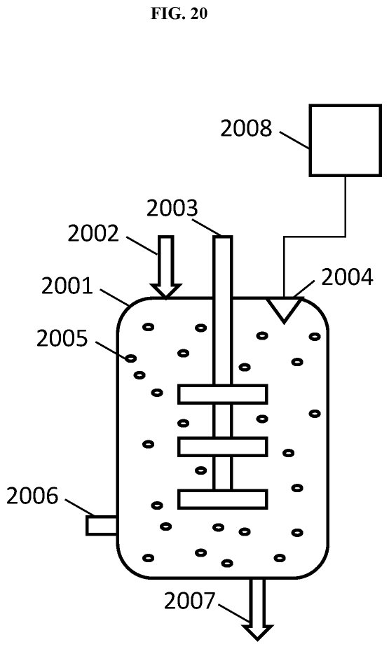

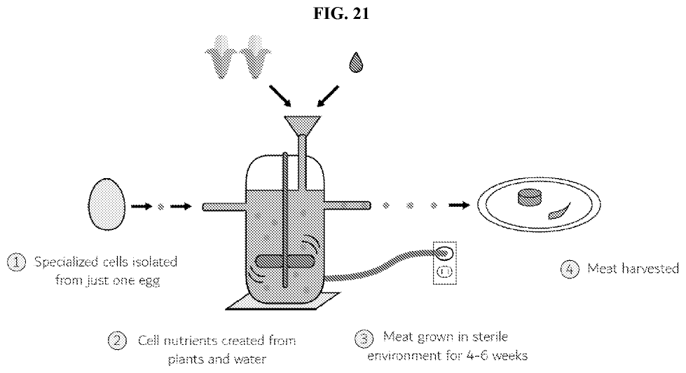

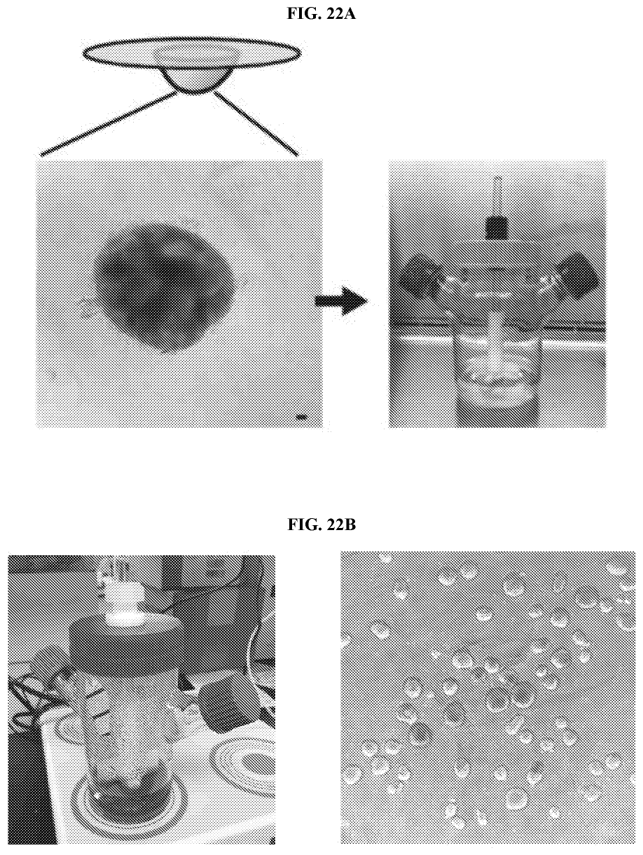

A bioreactor system is typically scalable for large-scale cell culture. FIG. 20 illustrates a diagram of one embodiment of a bioreactor system comprising a reactor chamber 2001 for culturing cells. Oftentimes, the bioreactor system comprises stirring element 2003 for agitation of the contents of the reactor chamber 2001. Continuous or periodic agitation helps keep cells, cell clumps, and/or micro-scaffolds in suspension. Fresh media is added into the reactor chamber via at least one input port 2002. Fresh media is sometimes maintenance media, differentiation media, steatotic media, proliferation media, or any other media formulation disclosed herein. Depleted media or effluent is removed from the reactor chamber via at least one output port 2007. In some cases, oxygen, carbon dioxide, and/or other gases are introduced through at least one input gas port 2006. The input gas port 2006 is optionally coupled to an aerator positioned inside the reactor chamber. Oftentimes, the bioreactor system comprises at least one sensor 2004 for monitoring the reactor chamber. The at least one sensor 2004 is usually in communication with a control unit 2008 (e.g. a computer). In many cases, the reactor chamber is seeded with a plurality of micro-scaffolds 2005. The micro-scaffolds 2005 enable adherence of certain adherent cells such as, for example, hepatocytes. For example, some methods of producing cultured fish meat for human consumption comprise: a) obtaining a population of self-renewing cells derived from fish; b) culturing the population of self-renewing cells in culture media comprising micro-scaffolds; c) inducing differentiation in the population of cells to form at least one of myocytes and adipocytes; and d) processing the population of cells into fish meat for human consumption.

FIG. 21 shows an illustrative process by which a bioreactor system is used for meat production. In this example, specialized cells such as embryonic, pluripotent, or multipotent cells are isolated from an egg and adapted for growth in the bioreactor (e.g. using a hanging drop method to form spheroid bodies as shown in FIG. 22). The cells are grown using media comprising water and nutrients created from plants (e.g. using a plant-based substitute for animal-derived serum such as soybean hydrolysate or mushroom extract). The cells are grown in the sterile environment of the bioreactor for 4-6 weeks. In some cases, the cells are differentiated, and then harvested and/or processed into a meat product.

Transitioning cells from 2-D cell culture plates to 3-D bioreactors can be carried out using various methods such as the hanging drop method shown in FIG. 22A. The hanging drop method entails placing cells in hanging drop culture and incubating them under physiological conditions until the cells form 3-D spheroids in which cells are in direct cell-cell contact and with extracellular matrix components. The illustrative example shown in FIG. 22A comprises suspending duck hepatocytes in hanging drops to initiate formation of spheroids (left panel). The spheroids are then transitioned into 3-D culture in a bioreactor (FIG. 22A right panel). The 3-D culture allows for more rapid proliferation and/or growth of the cultured cells. Another exemplary bioreactor is shown in FIG. 22B (left panel). The cells from the spheroids are capable of propagating in the 3-D culture (FIG. 22B right panel). In an exemplary embodiment of the hanging drop method, an adherent cell culture is washed with PBS and incubated with a 0.05% trypsin 1 mM EDTA solution to dissociate the cells. The trypsinization is then neutralized by addition of culture media, and the cells are digested with DNAse for 5 minutes at room temperature. The cells are centrifuged at 250 Gs for 5 minutes. Next, the supernatant is discarded, and the cells are resuspended in culture media. A hanging drop is formed by pipetting a volume (e.g. 10 μl) of culture media with cells onto the bottom of a lid from a tissue culture dish. Multiple hanging drops can be formed on a single lid. Several milliliters of PBS can be added to the bottom of the tissue culture dish to prevent dehydration of the hanging drops. The lid is then placed on the tissue culture dish followed by incubation at standard cell culture conditions (e.g. 5% CO₂, 37° C., 95% humidity) until spheroids are observed.

Sometimes, a bioreactor system comprises at least one bioreactor, bioreactor tank, or reactor chamber 2001. For example, in certain instances, a bioreactor system comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 reactor chambers. In some cases, a bioreactor system comprises about 1 reactor chamber to about 1,000 reactor chambers. Sometimes, a bioreactor system comprises about 1 reactor chamber. A bioreactor system usually comprises at most about 1,000 reactor chambers.

In some embodiments, a bioreactor system comprises about 1 reactor chamber to about 5 reactor chambers, about 1 reactor chamber to about 10 reactor chambers, about 1 reactor chamber to about 20 reactor chambers, about 1 reactor chamber to about 50 reactor chambers, about 1 reactor chamber to about 100 reactor chambers, about 1 reactor chamber to about 200 reactor chambers, about 1 reactor chamber to about 300 reactor chambers, about 1 reactor chamber to about 400 reactor chambers, about 1 reactor chamber to about 500 reactor chambers, about 1 reactor chamber to about 1,000 reactor chambers, about 5 reactor chambers to about 10 reactor chambers, about 5 reactor chambers to about 20 reactor chambers, about 5 reactor chambers to about 50 reactor chambers, about 5 reactor chambers to about 100 reactor chambers, about 5 reactor chambers to about 200 reactor chambers, about 5 reactor chambers to about 300 reactor chambers, about 5 reactor chambers to about 400 reactor chambers, about 5 reactor chambers to about 500 reactor chambers, about 5 reactor chambers to about 1,000 reactor chambers, about 10 reactor chambers to about 20 reactor chambers, about 10 reactor chambers to about 50 reactor chambers, about 10 reactor chambers to about 100 reactor chambers, about 10 reactor chambers to about 200 reactor chambers, about 10 reactor chambers to about 300 reactor chambers, about 10 reactor chambers to about 400 reactor chambers, about 10 reactor chambers to about 500 reactor chambers, about 10 reactor chambers to about 1,000 reactor chambers, about 20 reactor chambers to about 50 reactor chambers, about 20 reactor chambers to about 100 reactor chambers, about 20 reactor chambers to about 200 reactor chambers, about 20 reactor chambers to about 300 reactor chambers, about 20 reactor chambers to about 400 reactor chambers, about 20 reactor chambers to about 500 reactor chambers, about 20 reactor chambers to about 1,000 reactor chambers, about 50 reactor chambers to about 100 reactor chambers, about 50 reactor chambers to about 200 reactor chambers, about 50 reactor chambers to about 300 reactor chambers, about 50 reactor chambers to about 400 reactor chambers, about 50 reactor chambers to about 500 reactor chambers, about 50 reactor chambers to about 1,000 reactor chambers, about 100 reactor chambers to about 200 reactor chambers, about 100 reactor chambers to about 300 reactor chambers, about 100 reactor chambers to about 400 reactor chambers, about 100 reactor chambers to about 500 reactor chambers, about 100 reactor chambers to about 1,000 reactor chambers, about 200 reactor chambers to about 300 reactor chambers, about 200 reactor chambers to about 400 reactor chambers, about 200 reactor chambers to about 500 reactor chambers, about 200 reactor chambers to about 1,000 reactor chambers, about 300 reactor chambers to about 400 reactor chambers, about 300 reactor chambers to about 500 reactor chambers, about 300 reactor chambers to about 1,000 reactor chambers, about 400 reactor chambers to about 500 reactor chambers, about 400 reactor chambers to about 1,000 reactor chambers, or about 500 reactor chambers to about 1,000 reactor chambers.

In some cases, the at least one reactor chamber has an internal volume suitable for large-scale cell culture. In some cases, a reactor chamber has an internal volume of about 1 L to about 100,000 L. In most instances, a reactor chamber has an internal volume of at least about 1 L. Sometimes, a reactor chamber has an internal volume of at most about 100,000 L.

Oftentimes, a reactor chamber has an internal volume of about 1 L to about 10 L, about 1 L to about 50 L, about 1 L to about 100 L, about 1 L to about 500 L, about 1 L to about 1,000 L, about 1 L to about 5,000 L, about 1 L to about 10,000 L, about 1 L to about 50,000 L, about 1 L to about 100,000 L, about 10 L to about 50 L, about 10 L to about 100 L, about 10 L to about 500 L, about 10 L to about 1,000 L, about 10 L to about 5,000 L, about 10 L to about 10,000 L, about 10 L to about 50,000 L, about 10 L to about 100,000 L, about 50 L to about 100 L, about 50 L to about 500 L, about 50 L to about 1,000 L, about 50 L to about 5,000 L, about 50 L to about 10,000 L, about 50 L to about 50,000 L, about 50 L to about 100,000 L, about 100 L to about 500 L, about 100 L to about 1,000 L, about 100 L to about 5,000 L, about 100 L to about 10,000 L, about 100 L to about 50,000 L, about 100 L to about 100,000 L, about 500 L to about 1,000 L, about 500 L to about 5,000 L, about 500 L to about 10,000 L, about 500 L to about 50,000 L, about 500 L to about 100,000 L, about 1,000 L to about 5,000 L, about 1,000 L to about 10,000 L, about 1,000 L to about 50,000 L, about 1,000 L to about 100,000 L, about 5,000 L to about 10,000 L, about 5,000 L to about 50,000 L, about 5,000 L to about 100,000 L, about 10,000 L to about 50,000 L, about 10,000 L to about 100,000 L, or about 50,000 L to about 100,000 L.

In some cases, disclosed herein are bioreactor systems suitable for large-scale production of cultured cells for generation of food products. Oftentimes, cells are cultured on a batch basis. Alternatively, or in combination, cells are cultured continuously. In both batch and continuous cultures, fresh nutrients are usually supplied to ensure the appropriate nutrient concentrations for producing the desired food product. As an example, in a fed-batch culture, nutrients (e.g. fresh culture media) is supplied to the bioreactor, and the cultured cells remain in the bioreactor until they are ready for processing into the finished food product. In a semi-batch culture, a base media is supplied to the bioreactor and supports an initial cell culture, while an additional feed media is then supplied to replenish depleted nutrients. Sometimes, a bioreactor system produces at least a certain quantity of cells per batch. In some cases, a bioreactor system produces a batch of about 1 billion cells to about 100,000,000 billion cells. Oftentimes, a bioreactor system produces a batch of at least about 1 billion cells. A bioreactor system usually produces a batch of at most about 100,000,000 billion cells.

Sometimes, a bioreactor system produces a batch of about 1 billion cells to about 10 billion cells, about 1 billion cells to about 50 billion cells, about 1 billion cells to about 100 billion cells, about 1 billion cells to about 500 billion cells, about 1 billion cells to about 1,000 billion cells, about 1 billion cells to about 5,000 billion cells, about 1 billion cells to about 10,000 billion cells, about 1 billion cells to about 100,000 billion cells, about 1 billion cells to about 1,000,000 billion cells, about 1 billion cells to about 10,000,000 billion cells, about 1 billion cells to about 100,000,000 billion cells, about 10 billion cells to about 50 billion cells, about 10 billion cells to about 100 billion cells, about 10 billion cells to about 500 billion cells, about 10 billion cells to about 1,000 billion cells, about 10 billion cells to about 5,000 billion cells, about 10 billion cells to about 10,000 billion cells, about 10 billion cells to about 100,000 billion cells, about 10 billion cells to about 1,000,000 billion cells, about 10 billion cells to about 10,000,000 billion cells, about 10 billion cells to about 100,000,000 billion cells, about 50 billion cells to about 100 billion cells, about 50 billion cells to about 500 billion cells, about 50 billion cells to about 1,000 billion cells, about 50 billion cells to about 5,000 billion cells, about 50 billion cells to about 10,000 billion cells, about 50 billion cells to about 100,000 billion cells, about 50 billion cells to about 1,000,000 billion cells, about 50 billion cells to about 10,000,000 billion cells, about 50 billion cells to about 100,000,000 billion cells, about 100 billion cells to about 500 billion cells, about 100 billion cells to about 1,000 billion cells, about 100 billion cells to about 5,000 billion cells, about 100 billion cells to about 10,000 billion cells, about 100 billion cells to about 100,000 billion cells, about 100 billion cells to about 1,000,000 billion cells, about 100 billion cells to about 10,000,000 billion cells, about 100 billion cells to about 100,000,000 billion cells, about 500 billion cells to about 1,000 billion cells, about 500 billion cells to about 5,000 billion cells, about 500 billion cells to about 10,000 billion cells, about 500 billion cells to about 100,000 billion cells, about 500 billion cells to about 1,000,000 billion cells, about 500 billion cells to about 10,000,000 billion cells, about 500 billion cells to about 100,000,000 billion cells, about 1,000 billion cells to about 5,000 billion cells, about 1,000 billion cells to about 10,000 billion cells, about 1,000 billion cells to about 100,000 billion cells, about 1,000 billion cells to about 1,000,000 billion cells, about 1,000 billion cells to about 10,000,000 billion cells, about 1,000 billion cells to about 100,000,000 billion cells, about 5,000 billion cells to about 10,000 billion cells, about 5,000 billion cells to about 100,000 billion cells, about 5,000 billion cells to about 1,000,000 billion cells, about 5,000 billion cells to about 10,000,000 billion cells, about 5,000 billion cells to about 100,000,000 billion cells, about 10,000 billion cells to about 100,000 billion cells, about 10,000 billion cells to about 1,000,000 billion cells, about 10,000 billion cells to about 10,000,000 billion cells, about 10,000 billion cells to about 100,000,000 billion cells, about 100,000 billion cells to about 1,000,000 billion cells, about 100,000 billion cells to about 10,000,000 billion cells, about 100,000 billion cells to about 100,000,000 billion cells, about 1,000,000 billion cells to about 10,000,000 billion cells, about 1,000,000 billion cells to about 100,000,000 billion cells, or about 10,000,000 billion cells to about 100,000,000 billion cells.

In some cases, a bioreactor system produces a batch of cultured cells during a certain time period. For example, in some cases, a bioreactor system produces a batch of cultured cells at least once every 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In some cases, a bioreactor system produces a batch of cultured cells having at least a certain mass. Sometimes, the mass is measured as dry weight with excess media or supernatant removed. Usually, a bioreactor system produces a batch of cultured cells of about 1 kg to about 10,000 kg. In certain instances, a bioreactor system produces a batch of at least about 1 kg. Oftentimes, a bioreactor system produces a batch of at most about 10,000 kg.

In certain instances, a bioreactor system produces a batch of about 1 kg to about 5 kg, about 1 kg to about 10 kg, about 1 kg to about 20 kg, about 1 kg to about 30 kg, about 1 kg to about 40 kg, about 1 kg to about 50 kg, about 1 kg to about 100 kg, about 1 kg to about 500 kg, about 1 kg to about 1,000 kg, about 1 kg to about 5,000 kg, about 1 kg to about 10,000 kg, about 5 kg to about 10 kg, about 5 kg to about 20 kg, about 5 kg to about 30 kg, about 5 kg to about 40 kg, about 5 kg to about 50 kg, about 5 kg to about 100 kg, about 5 kg to about 500 kg, about 5 kg to about 1,000 kg, about 5 kg to about 5,000 kg, about 5 kg to about 10,000 kg, about 10 kg to about 20 kg, about 10 kg to about 30 kg, about 10 kg to about 40 kg, about 10 kg to about 50 kg, about 10 kg to about 100 kg, about 10 kg to about 500 kg, about 10 kg to about 1,000 kg, about 10 kg to about 5,000 kg, about 10 kg to about 10,000 kg, about 20 kg to about 30 kg, about 20 kg to about 40 kg, about 20 kg to about 50 kg, about 20 kg to about 100 kg, about 20 kg to about 500 kg, about 20 kg to about 1,000 kg, about 20 kg to about 5,000 kg, about 20 kg to about 10,000 kg, about 30 kg to about 40 kg, about 30 kg to about 50 kg, about 30 kg to about 100 kg, about 30 kg to about 500 kg, about 30 kg to about 1,000 kg, about 30 kg to about 5,000 kg, about 30 kg to about 10,000 kg, about 40 kg to about 50 kg, about 40 kg to about 100 kg, about 40 kg to about 500 kg, about 40 kg to about 1,000 kg, about 40 kg to about 5,000 kg, about 40 kg to about 10,000 kg, about 50 kg to about 100 kg, about 50 kg to about 500 kg, about 50 kg to about 1,000 kg, about 50 kg to about 5,000 kg, about 50 kg to about 10,000 kg, about 100 kg to about 500 kg, about 100 kg to about 1,000 kg, about 100 kg to about 5,000 kg, about 100 kg to about 10,000 kg, about 500 kg to about 1,000 kg, about 500 kg to about 5,000 kg, about 500 kg to about 10,000 kg, about 1,000 kg to about 5,000 kg, about 1,000 kg to about 10,000 kg, or about 5,000 kg to about 10,000 kg.

Cells grown in bioreactor systems are typically grown in suspension. Oftentimes, the bioreactor system has components that enable the automated growth and maintenance of cell cultures. A bioreactor comprises at least one reactor chamber or reactor tank wherein the cultured cells grow. In some cases, the bioreactor system has at least one pump for circulating the media, introducing fresh media, and/or removing waste media. Oftentimes, the bioreactor system comprises a plurality of media tanks for introducing various types of media such as, for example, proliferation media, maintenance media (e.g. for maintenance of self-renewal ability), differentiation media, and steatotic media. Sometimes, the bioreactor system comprises at least one of oxygenator(s), carbon dioxide regulator(s), and a central control unit regulating components of the bioreactor system. In many instances, the bioreactor has a stirring element for maintaining cultured cells in suspension and/or keeping the media mixed. The bioreactor usually comprises at least one sensor for monitoring the environment inside the reactor chamber. A sensor is typically a biosensor, a chemosensor, or an optical sensor for monitoring parameters important to cell culture. In some cases, a sensor is configured to monitor at least one of pH, temperature, oxygen, carbon dioxide, glucose, lactate, ammonia, hypoxanthine, amino acid(s), dopamine, and lipid(s). Oftentimes, the at least one sensor is in communication with a control unit (e.g. a computer system) that monitors the sensor parameters. In certain instances, the control unit provides the sensor parameters to a user such as, for example, on a display screen. The control unit often comprises at least one input source for receiving commands from a user.

In some cases, cells are cultured in suspension in cell culture flasks. The cell culture flasks are optionally stacked and/or arranged side-by-side as described for the 2D surface cell culture. Cells cultured in suspension are usually non-adherent cells. In some cases, however, adherent cells are cultured on scaffolds in a suspension. Scaffolds provide structural support and a physical environment for cells to attach, grow, and migrate. In addition, scaffolds usually confer mechanical properties such as elasticity and tensile strength. Oftentimes, 3D scaffolds are used to culture adherent cells so as to enable 3D growth of the cells. Scaffolds sometimes have specific shapes or sizes for guiding the growth of the cultured cells. In some cases, scaffolds are composed of one or more different materials. Some scaffolds are solid scaffolds, while others are porous. Porous scaffolds allow cell migration or infiltration into the pores. Scaffolds are typically composed of a biocompatible material to induce the proper recognition from cells. In addition, the scaffold is made of a material with suitable mechanical properties and degradation kinetics for the desired tissue type that is generated from the cells. In certain instances, a scaffold comprises a degradable material to enable remodeling and/or elimination of the scaffold in the cultured food product. For example, in some cases, a 3D scaffold that shapes cultured hepatocytes into the shape of a liver biodegrades after the hepatocytes expand to fill up the interior space of the scaffold. In other instances, the scaffold comprises a material that remains in the cultured food product. For example, sometimes, at least a portion of a collagen scaffold providing support to cultured myocytes remains to provide texture and continuing structural support in the cultured food product. In some cases, a scaffold comprises a hydrogel, a biomaterial such as extracellular matrix molecule (ECM) or chitosan, or biocompatible synthetic material (e.g. polyethylene terephthalate). ECM molecules are typically proteoglycans, non-proteoglycan polysaccharides, or proteins. Potential ECM molecules for use in scaffolding include collagen, elastin, heparan sulfate, chondroitin sulfate, keratan sulfate, hyaluronic acid, laminin, and fibronectin. Sometimes, plant-based scaffolds are used for 3D culturing. Non-limiting examples of plant-based scaffolds include scaffolds obtained from plants such as apples, seaweed, or jackfruit. The plant-based scaffolds often comprise at least one plant-based material such as cellulose, hemicellulose, pectin, lignin, alginate, or any combination thereof. Sometimes, plant-based scaffolds are decellularized. In some cases, scaffolds are not required for 3D culturing. In various instances, scaffolds used in the methods and compositions described herein comprise at least one of hydrogel, chitosan, polyethylene terephthalate, collagen, elastin, heparan sulfate, chondroitin sulfate, keratan sulfate, hyaluronic acid, laminin, fibronectin, cellulose, hemicellulose, pectin, lignin, alginate, glucomannan, polycaprolactone (PCL), textured vegetable protein (TVP), and acrylates. An example of textured vegetable protein is textured soy protein (TSP), which typically comprises a high percentage of soy protein, soy flour, or soy concentrate. TVP and TSP can be used to provide a meat-like texture and consistency to the meat products described herein. In some cases, the meat product comprising TVP or TSP is seasoned to taste like meat (e.g., using various salts, herbs, and/or spices).

In some cases, cells are cultured with micro-scaffolds that enable cell adhesion. Micro-scaffolds are usually Micro-scaffolds allow adherent cells to be grown in a suspension bioreactor system. For example, a micro-scaffold provides a surface for adherent cells such as hepatocytes to attach even while the micro-scaffold itself is in suspension. In certain instances, scaffolds and/or micro-scaffolds are produced by 3D printing of an appropriate material (e.g. collagen). Micro-scaffolds often have a porous structure. Sometimes, a micro-scaffold has a solid non-porous structure. A micro-scaffold is smaller than a conventional scaffold. Scaffolds are typically used for providing macroscopic structure and/or shape for the cell population, whereas a micro-scaffold usually provides a seed or core structure for adherent cells to attach while remaining small enough to remain in suspension with stirring. The use of micro-scaffolds enables the culturing of adherent cells in a suspension culture. The culturing of cells using micro-scaffolds in a bioreactor system suspension culture enables the large-scale production of adherent cells. For example, adherent cells such as hepatocytes, myocytes, and adipocytes are capable of being grown on a large scale in bioreactor suspension cultures using micro-scaffolds. This allows for the production of luxury food items like foie gras or sushi grade salmon meat. Alternatively, cultured fish cells are sometimes processed into surimi such as salmon, tuna, or trout surimi.

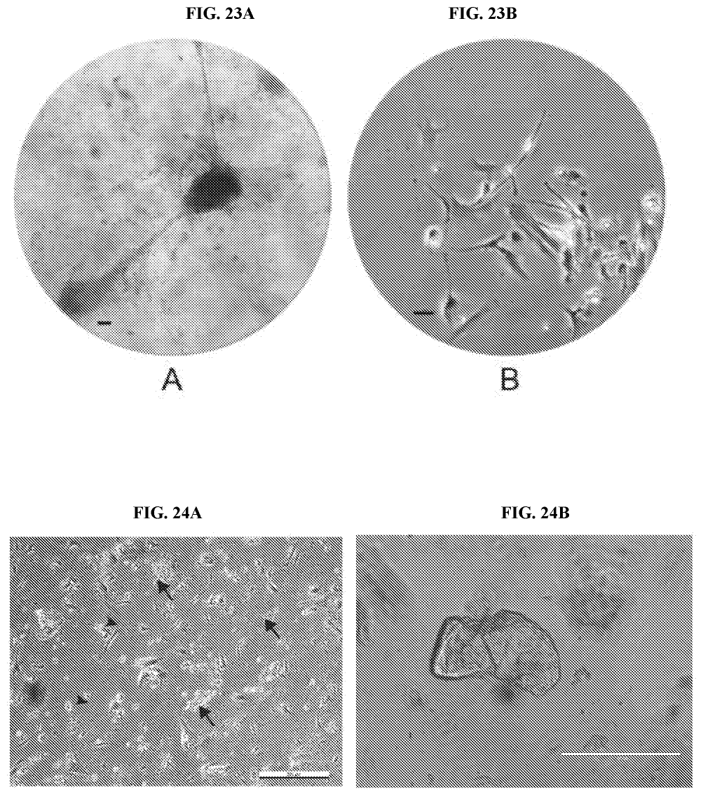

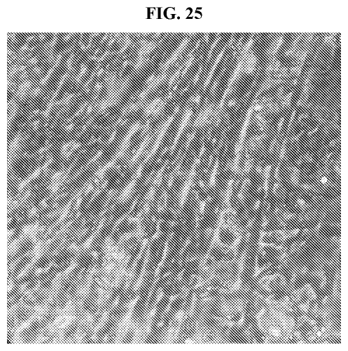

FIG. 23A shows trout myosatellite cells grown on glucomannan microscaffolds that have successfully differentiated into myotubes in the 3-dimensional culture. FIG. 23B shows a negative control of undifferentiated myosatellite cells. FIG. 24A shows duck fibroblasts (arrowheads) successfully grown on glucomannan microscaffolds (arrows). FIG. 24B shows a representative glucomannan microscaffold. Alternative materials can be used in producing microscaffolds. Microscaffolds can comprise at least one of glucomannan, alginate, chitosan, polycaprolactone (PCL), matrix proteins (e.g., collagen, fibronectin, laminin), textured vegetable protein (TVP), textured soy protein (TSP), and acrylates. In some cases, polycaprolactone (PCL) is used to generate PCL-woven scaffolds or PCL-fibrin composites. In some cases, the microscaffolds are modified. In certain instances, microscaffolds are conjugated to one or more factors relevant to cell attachment, growth, differentiation, or a combination thereof. As an example, glucomannan microscaffolds are conjugated to FGF2 to promote growth and differentiation of myosatellite cells into myotubes. In some cases, microscaffolds are conjugated to one or more growth or differentiation factors via covalent bonding such as chemical cross-linking or other reactions for immobilizing the factors to the microscaffold structure.

Alternatively, in some cases, scaffolds or micro-scaffolds are not needed for culturing of cells in suspension. Sometimes, the cells are non-adherent cells and do not require a substrate or surface for attachment. In certain instances, the cells have been modified or engineered to no longer require an adherence substrate. For example, hepatocytes are normally adherent cells, but in some instances, hepatocytes are modified to no longer require an extracellular matrix for attachment for survival and proliferation. Other adherent cells include myocytes and adipocytes, which are sometimes cultured to produce cultured meat products. For example, some methods of producing cultured meat for human consumption comprise: a) obtaining a population of self-renewing cells, said cells capable of growing in suspension culture; b) culturing the population of self-renewing cells in suspension; c) inducing differentiation in the population of cells to form at least one of myocytes and adipocytes; and d) processing the population of cells into meat for human consumption.

The cells culturing systems described herein enable the culturing of cells for food production in a pathogen-free environment. Generally, cells are grown in a culture environment free of dangerous contaminants that affect human health. Cell culture plates, flasks, and bioreactors typically provide cell culture conditions free of dangerous pathogens (e.g. H1N1), parasites, heavy metals, and toxins (e.g. bacterial endotoxins, pesticides, etc.). In some cases, the methods described herein do not utilize antibiotics. Sometimes, the methods use an inducing agent such as tetracycline for a one-time induction of cell differentiation and/or a cell phenotype, followed by removal of the inducing agent before the cells are processed into a food product.

### Tissue Processing

Provided herein are systems and methods for processing cultured cells to create an appropriate taste, texture, consistency, or other desired quality in a food product. The cells are typically a differentiated cell population such as, for example, myocytes, adipocytes, or hepatocytes.

In various cases, the cells are animal cells. Sometimes, the cells are fish, animal, or avian cells. Examples of avian cells include cells derived from geese, ducks, chickens, Cornish game hens, pheasants, turkeys, Guinea hens, quails, pigeons, partridges, emus, ostriches, capons, grouses, swans, doves, woodcocks, chukars, and snipes. As an example, FIG. 25 shows an image of duck muscle tissue created according to the methods disclosed herein. The successful generation of the duck muscle tissue in FIG. 25 was confirmed by the muscle tissue's ability to spontaneously contract.

Some methods disclosed herein allow production of cultured non-textured muscle tissue for human consumption. Non-textured muscle tissue includes certain fish muscle tissues. Certain methods of generating non-textured muscle tissue comprise: a) obtaining a population of self-renewing cells; b) culturing the population of self-renewing cells; c) inducing differentiation in the population of cells to form non-textured muscle tissue; and d) processing the cultured non-textured muscle tissue for human consumption. In addition, some methods produce cultured fish tissue having enhanced nutritional content for human consumption. For example, certain methods comprise: a) culturing a population of fish myocytes in a culture media having at least one nutritional supplement; b) expanding the population of myocytes; and c) processing the population of myocytes into fish tissue for human consumption. The systems and methods described herein often allow production of edible compositions comprising fish tissue produced from cultured myocytes and adipocytes.

In some cases, the fish adipocytes and/or myocytes are processed into fish meat such as, for example, salmon meat. In various instances, fish adipocytes and/or myocytes are processed into a finished fish meat product. Other examples of processed fish products include minced fish meat, fish fillet, fish cutlet, and fish steak. These various shapes and sizes of the fish product are obtained by processing the myocytes and/or adipocytes together with various additional ingredients such as a binder, filler, or extender to provide structural cohesion and/or texture. In some instances, the meat product is cooked or cured. Processing the cultured cells into a meat product can include at least one of smoking, fermenting, salting, marinating, poaching, baking, barbecuing, casseroling, shallow frying, deep frying, oven frying, grilling, and microwaving. In some cases, the cells are processed into sushi grade fish meat suitable for raw consumption without being frozen. In certain cases, the fish meat is not cooked during processing. Examples of sushi grade fish meat produced according to the systems and methods disclosed herein include salmon and tuna. As used herein, sushi grade meat refers to meat that is produced free of parasites and bacteria. The cells are usually free of pathogens, parasites, toxins, heavy metals (e.g. mercury), antibiotics, or any combination thereof. Certain systems and methods described herein provide for the production of cultured food products without exposure to contaminant(s). Some methods enable production of cultured cells for human consumption without using antibiotics. For example, certain methods comprise: a) culturing a population of cells without using antibiotics; b) inducing differentiation within the population of cells; c) inducing high lipid accumulation within the population of cells; and d) processing the population of cells for human consumption. Also disclosed herein are methods of producing cultured cells for human consumption without exposure to pathogens. Some such methods comprise: a) culturing a population of cells in a pathogen-free culture environment; b) inducing differentiation within the population of cells; c) inducing high lipid accumulation within the population of cells; and d) processing the population of cells for human consumption. Certain methods allow cultured food production without exposure to toxins. For example, some such methods comprise: a) culturing a population of cells in a toxin-free culture environment; b) inducing differentiation within the population of cells; c) inducing high lipid accumulation within the population of cells; and d) processing the population of cells for human consumption.

In certain cases, cultured meat comprises a mixed population of myocytes and adipocytes. Oftentimes, pre-adipocytes and satellite cells are isolated from a source such as, for example, fish fingerlings. The pre-adipocytes and satellite cells are useful because they have some self-renewal capacity. The pre-adipocytes and satellite cells are typically cultured and expanded, and subsequently differentiated. In some cases, the pre-adipocytes and satellite cells are cultured together. Usually, they are cultured separately until after differentiation when they are co-cultured together at a certain ratio to produce a desired ratio in a final fish product. Alternatively, a population of cells is sometimes induced to differentiate into different cell types in the same culture. For example, in this scenario, some cells form into adipocytes and some form into myocytes. Usually, myocytes and adipocytes are cultured separately, and subsequently mixed. Sometimes, the myocytes and adipocytes are homogeneously mixed in equal proportions. In other cases, the myocytes and adipocytes are heterogeneously mixed in unequal proportions. For co-culturing or processing, the myocytes and adipocytes are typically combined at a certain ratio or proportion. For example, in some cases, myocytes and adipocytes are combined at a ratio of at least 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 35:1, 40:1, 45:1, 50:1, 60:1, 70:1, 80:1, 90:1, or at least 100:1, respectively. Oftentimes, the myocytes and/or adipocytes are fish cells. In some instances, the myocytes and/or adipocytes are derived from salmon. Sometimes, the myocytes and/or adipocytes are derived from sea bass, tuna, mackerel, blue marlin, swordfish, yellowtail, salmon, trout, eel, abalone, squid, clams, ark shell, sweetfish, scallop, sea bream, halfbeak, shrimp, flatfish, cockle, octopus, or crab. Examples of tuna include yellowfin, southern Bluefin, northern Bluefin, Thunnus alalunga, Thunnus atlanticus, and Thunnus obesus. In certain cases, instead of combining myocytes and adipocytes, myocytes are induced to undergo steatosis to provide the desired lipid or fat content found in conventional salmon meat without requiring adipocytes.

Provided herein are systems and methods for producing meat having a certain ratio of fast twitch and slow twitch muscle cells and/or fibers. The meat produced according to the systems and methods disclosed herein usually comprises myocytes or skeletal muscle cells having a certain ratio or proportion of fast twitch (type II) and slow twitch (type I) muscle fibers. Slow twitch muscle fibers exhibit low-intensity contractions fueled by the oxidative pathway and demonstrate relatively higher endurance, while fast twitch muscle fibers have higher intensity contractions fueled by the glycolytic pathway. Fast twitch muscles are characterized by high glycolytic and anaerobic muscle fibers. The ratio of fast twitch and slow twitch muscle fibers in muscle tissue plays a role in the taste, color, texture, and other culinary properties of the meat. For example, fish meat is characterized by a high proportion of fast twitch muscle fibers compared to animal meat, which has some role in the culinary differences between the two categories of meat. Sometimes, myocytes such as salmon myocytes are cultured to comprise at least 10%, 20%, 30%, 40%, 50%, 60%, 70%, 80%, 90%, 95% or more fast twitch fibers (e.g. high glycolytic and anaerobic muscle fibers). The percentage of fast twitch muscle fibers can be characterized by evaluating tissue samples using various laboratory techniques such as microscopy-based imaging (e.g. staining tissue sections for fast twitch muscle fiber markers). In some cases, a myocyte generated according to the methods described herein has a length of at least about 10 μm, about 20 μm, about 30 μm, about 40 μm, about 50 μm, about 60 μm, about 70 μm, about 80 μm, about 90 μm, about 100 μm, about 110 μm, about 120 μm, about 130 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1000 μm, about 2000 μm, about 3000 μm, about 4000 μm, about 5000 μm, about 6000 μm, about 7000 μm, about 8000 μm, about 9000 μm, or about 10000 μm or more.

Certain muscle tissues lack the texture of animal skeletal muscle such as beef, pork, or chicken. For example, fish muscle tissue tends to have a non-textured or un-textured consistency. Fish muscle tissue such as salmon and tuna is often described as having a tasted texture that is delicate, soft, and/or having a homogeneous consistency. Other examples of non-textured meat include squid and octopus muscle tissue, which have a distinct non-textured consistency in comparison to animal skeletal muscle. Liver food products such as foie gras also have a non-textured characteristic. Certain methods described herein allow production of cultured non-textured tissue. Some such methods comprise: a) culturing a population of cells; b) inducing differentiation in the population of cells; c) manipulating lipid metabolic pathways to induce steatosis in the population of cells such that the cells accumulate high lipid content; and d) processing the population of cells into non-textured tissue.

Certain methods disclosed herein produce cultured non-muscle tissue for human consumption. Some such methods comprise: a) obtaining a population of self-renewing cells; b) culturing the population of self-renewing cells; c) inducing differentiation in the population of cells to form non-muscle tissue; and d) processing the cultured non-muscle tissue for human consumption. In some cases, the cells are hepatocytes. The hepatocytes are harvested after culturing for processing into a food product. Sometimes, the hepatocytes are processed into foie gras.

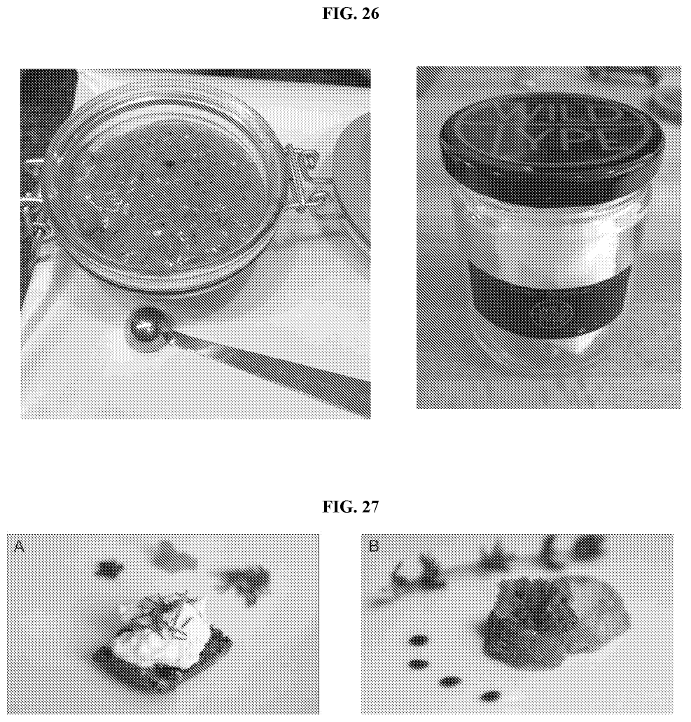

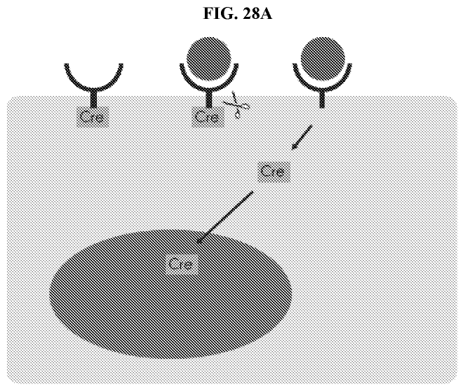

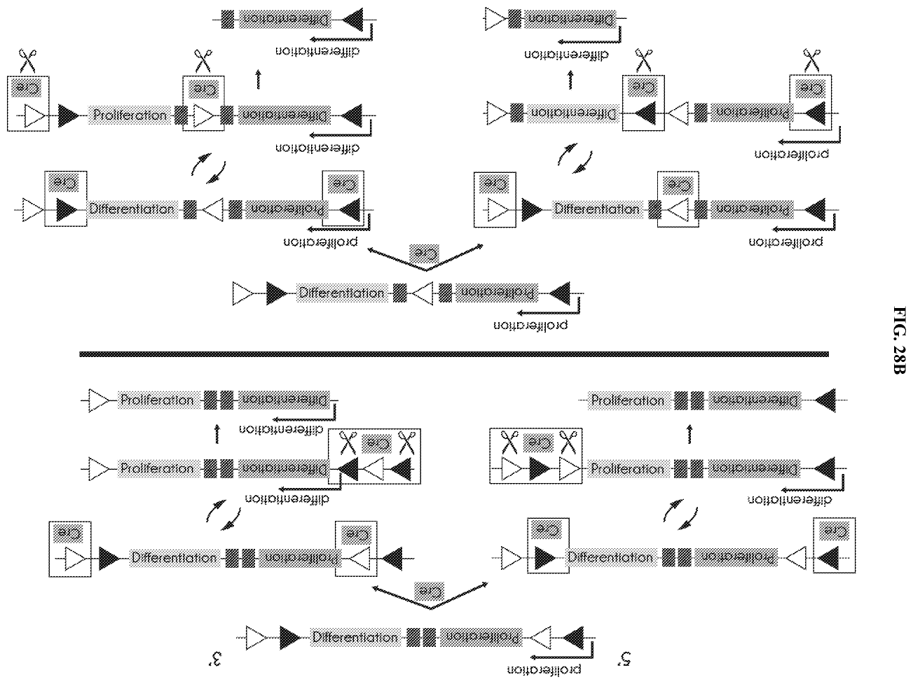

The systems and methods disclosed herein enable the production of culinary foie gras compositions comprising tissue cultured hepatocytes having high lipid content and processed for human consumption. Some food product compositions comprise cultured organ cells processed into a non-textured non-muscle food product for human ingestion. The food product is sometimes an edible foie gras composition comprising cultured steatotic avian liver cells and seasoning. Oftentimes, foie gras compositions comprise cultured liver cells having high lipid content and liver cells having low lipid content. Edible compositions are sometimes produced that comprise avian liver cells grown in cell culture and processed for human consumption. In some cases, the food product is packaged with an optional label. As an example, some packaged foie gras compositions comprise cultured liver cells and packaging having a label indicating the foie gras composition was produced without forced feeding. Other packaged foie gras compositions comprise cultured liver cells processed into foie gras and packaging having a label indicating the foie gras was produced in a pathogen-free environment. In certain aspects, packaged edible compositions comprise cultured cells processed into a food product and packaging having a label indicating the composition was produced without exposure to a toxin. FIG. 26 shows exemplary food products produced from duck hepatocytes. The left panel shows a duck liver pâté made using duck steatotic liver cells. The right panel shows fois gras butter made using duck steatotic liver cells. FIG. 27 shows exemplary food products for human consumption produced according to the methods disclosed herein. The left panel shows a salmon pâté produced using salmon myocytes. The right panel shows a duck meat pâté produced using duck myocytes. In addition, chicken meat pâtés have also been developed using chicken myocytes.

In some cases, the foie gras has a substantially identical texture and/or consistency with conventional foie gras. In many cases, the foie gras is rated as grade A, grade B, or grade C foie gras. The foie gras has no blemishes, in some cases. The foie gras usually has no more than 1, 2, 3, 4, 5, 6, 7, 8, 9, or 10 blemishes. Sometimes, the hepatocytes are processed as a single foie gras composition. For example, a single foie gras composition is typically a liver or liver-shaped. Methods of processing harvested cells for human consumption include centrifugation and compaction. In some cases, the harvested cells receive some degree of structural integrity from the scaffold and/or micro-scaffolds on which the cells are attached during culturing. Sometimes, the harvested cells are combined with at least one other ingredient. The harvested cells are often combined with at least one other ingredient to obtain a food product having a desired texture, moisture retention, product adhesion, or any combination thereof. An ingredient is typically a binder, filler, or extender. A filler or binder is oftentimes a non-meat substance comprising carbohydrates such as a starch. Examples of fillers and binders include potato starch, flour, eggs, gelatin, carrageenan, and tapioca flour. Alternatively, extenders tend to have high protein content. Examples of extenders include soy protein, milk protein, and meat-derived protein. Certain ingredients that provide flavor, texture, or other culinary properties are added in some instances. For example, sometimes, extracellular matrix proteins are used to modulate structural consistency and texture. Certain proteins such as heme and collagen are occasionally incorporated into the extracellular matrix to contribute to the taste and texture of the final food product.

In many cases, cells are grown in suspension culture on micro-scaffolds that comprise at least one natural protein with texture-modifying properties. Micro-scaffolds of varying compositions can be used to produce a desired texture and/or consistency in the final food product. Sometimes, textured vegetable protein such as soy protein is used. Micro-scaffolds optionally comprise at least one filler or binder material for providing texture to the food product. Sometimes, micro-scaffolds are made of materials that biodegrade such that the finished food product no longer has any micro-scaffold structures remaining. For example, a population of cells is seeded onto micro-scaffolds in a bioreactor. As the cells adhere to the micro-scaffolds and proliferate, the micro-scaffolds gradually biodegrade until all that remains are the clumps of cells that are now adhered to each other and the extracellular matrix materials that they have secreted. Accordingly, micro-scaffolds (and also larger 3-D scaffolds) can be used to guide the structure of the resulting cultured food product but do not remain in the food product for consumption by a human. Alternatively, micro-scaffolds and 3-D scaffolds may comprise materials that do not biodegrade and/or remain in the cultured food product for consumption. For example, certain materials described herein can be used to generate the scaffolds in order to confer a particular structure, texture, taste, or other desired property.

In certain instances, a foie gras composition weighs at least 1, 2, 3, 4, 5, 6, 7, 8, 9, 10, 12, 16, 20, 24, 28, 32, 36, 42, 48, 52, 56, 60, or 64 ounces. Oftentimes, a foie gras composition weighs about 1 ounce to about 64 ounces. In many cases, a foie gras composition weighs at least about 1 ounce. A foie gras composition usually weighs at most about 64 ounces.

In some embodiments, a foie gras composition weighs about 1 ounce to about 2 ounces, about 1 ounce to about 4 ounces, about 1 ounce to about 8 ounces, about 1 ounce to about 12 ounces, about 1 ounce to about 16 ounces, about 1 ounce to about 20 ounces, about 1 ounce to about 24 ounces, about 1 ounce to about 30 ounces, about 1 ounce to about 36 ounces, about 1 ounce to about 48 ounces, about 1 ounce to about 64 ounces, about 2 ounces to about 4 ounces, about 2 ounces to about 8 ounces, about 2 ounces to about 12 ounces, about 2 ounces to about 16 ounces, about 2 ounces to about 20 ounces, about 2 ounces to about 24 ounces, about 2 ounces to about 30 ounces, about 2 ounces to about 36 ounces, about 2 ounces to about 48 ounces, about 2 ounces to about 64 ounces, about 4 ounces to about 8 ounces, about 4 ounces to about 12 ounces, about 4 ounces to about 16 ounces, about 4 ounces to about 20 ounces, about 4 ounces to about 24 ounces, about 4 ounces to about 30 ounces, about 4 ounces to about 36 ounces, about 4 ounces to about 48 ounces, about 4 ounces to about 64 ounces, about 8 ounces to about 12 ounces, about 8 ounces to about 16 ounces, about 8 ounces to about 20 ounces, about 8 ounces to about 24 ounces, about 8 ounces to about 30 ounces, about 8 ounces to about 36 ounces, about 8 ounces to about 48 ounces, about 8 ounces to about 64 ounces, about 12 ounces to about 16 ounces, about 12 ounces to about 20 ounces, about 12 ounces to about 24 ounces, about 12 ounces to about 30 ounces, about 12 ounces to about 36 ounces, about 12 ounces to about 48 ounces, about 12 ounces to about 64 ounces, about 16 ounces to about 20 ounces, about 16 ounces to about 24 ounces, about 16 ounces to about 30 ounces, about 16 ounces to about 36 ounces, about 16 ounces to about 48 ounces, about 16 ounces to about 64 ounces, about 20 ounces to about 24 ounces, about 20 ounces to about 30 ounces, about 20 ounces to about 36 ounces, about 20 ounces to about 48 ounces, about 20 ounces to about 64 ounces, about 24 ounces to about 30 ounces, about 24 ounces to about 36 ounces, about 24 ounces to about 48 ounces, about 24 ounces to about 64 ounces, about 30 ounces to about 36 ounces, about 30 ounces to about 48 ounces, about 30 ounces to about 64 ounces, about 36 ounces to about 48 ounces, about 36 ounces to about 64 ounces, or about 48 ounces to about 64 ounces.

In some instances, cultured cells are processed into additional food products aside from foie gras and salmon or fish meat. For example, the cells are sometimes processed into chopped or whole liver for culinary purposes. In some cases, other tissues are generated for human consumption such as, for example, yakitori or other chicken organ products. Oftentimes, other organs are generated for avian and other species (e.g., thymus or pancreas for sweet breads). In some cases, fatty or steatotic hepatocytes are generated and blended with healthy liver cells to make foie gras pâté or other terrines. The stem cell isolation techniques described herein are optionally used for the purpose of growing chicken, duck meat, or those of other animals. In certain instances, the techniques described herein are also applicable to the production of other animal-based meats.

Some of the systems and methods disclosed herein allow production of cultured liver cells for human consumption. Certain methods enable production of cultured non-textured tissue having high lipid content, the methods comprising: a) culturing a population of cells; b) inducing differentiation in the population of cells; c) manipulating lipid metabolic pathways to induce steatosis in the population of cells such that the cells accumulate high lipid content; and d) processing the population of cells into non-textured tissue having high lipid content.

Certain cultured cells and/or tissues produced using the methods described herein are processed into food products. In some cases, the cultured food product is packaged and/or labeled. The cultured cells and/or tissues can be processed into a plurality of slices (e.g. slices of foie gras or salmon meat) to form the cultured food product. The plurality of slices can be at least 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 slices. Sometimes, the plurality of slices is no more than 2, 3, 4, 5, 6, 7, 8, 9, 10, 11, 12, 13, 14, 15, 16, 17, 18, 19, 20, 25, 30, 35, 40, 45, 50, 60, 70, 80, 90, or 100 slices. In certain instances, the cultured food product is processed into shaped portions. The portions may be individually packaged or packaged together. The portions may be any number of shapes such as rectangle, square, circular, triangle, doughnut, tube, pyramid, or other shapes. The portions can have a flat shape (e.g. thin slices). For example, a portion of the cultured food product can have a thickness of no more than about 5 mm, about 10 mm, about 20 mm, about 30 mm, about 40 mm, about 50 mm, about 60 mm, about 70 mm, about 80 mm, about 90 mm, or about 100 mm. Sometimes, a portion of the cultured food product has a thickness of at least about 5 mm, about 10 mm, about 20 mm, about 30 mm, about 40 mm, about 50 mm, about 60 mm, about 70 mm, about 80 mm, about 90 mm, or about 100 mm.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### DETAILED DESCRIPTION OF FIGURES

FIG. 1 illustrates one embodiment of a process for producing cells for human consumption using self-renewing cells. In this process, the population of self-renewing cells is obtained 101, and then cultured 102 (e.g., on microscaffolds). Differentiation is induced in the cell population 103 followed by induction of lipid accumulation 104. Finally, the population of cells is processed for human consumption 105.

FIG. 2 illustrates one embodiment of a process for culturing muscle tissue for human consumption. In this example, a first and a second population of self-renewing cells are obtained 201, 204. The two populations of cells are cultured 202, 205 to the desired population size. Next, differentiation is induced in the two populations to generate myocytes 203 and adipocytes 206. Finally, the two populations of cells are processed for human consumption 207.

FIG. 3 shows an overview of an exemplary process for preparing cultured meat for consumption. First, stem cell identification, isolation, and characterization are carried out. These cells are then grown in two-dimensional culture such as on a feeder cell layer. These cells are transitioned into suspension culture in a bioreactor. Subsequent to transitioning to suspension culture, the cells are differentiated into muscle cells. The meat is then harvested, and finally prepared and cooked.

FIG. 4A shows illustrative approaches for growing a meat product for human consumption with avian hepatocytes as an exemplary example for making foie gras. First, the initial cell line is obtained. In one approach, embryonic stem cells are isolated from avian embryos 401. Induced pluripotent stem cells can also be generated and differentiated from avian dermal fibroblasts 402. Embryonic germ cells can also be isolated from avian embryos 403. Direct reprogramming of avian dermal fibroblasts to hepatocytes is an option 404. Another method entails immortalization of adult differentiated avian hepatocytes 405 such as by viral transduction. In some cases, adult differentiated avian hepatocytes can be serially passaged and grown, and selected for spontaneously transformed cells (e.g. spontaneously immortalized cells due to random mutation) 406. Nascent hepatic stem cells in adult avian liver tissue can also be isolated 407. In addition, cultured hepatocytes can be cultured and subjected to toxin or injury to enhance proliferative potential 408. The isolated stem cells 411 are then differentiated into differentiated hepatocytes 412 and subjected to induction of steatosis 413. Steatosis can be induced using a variety of methods including genetic intervention and exogenous treatments 409. Finally, these processes are assessed for scaling strategies 410 to enable large-scale production.

FIG. 4B shows illustrative approaches for growing a fish meat product for human consumption. First, the initial cell line is obtained. In one approach, embryonic stem cells are isolated from fish embryos 421. Induced pluripotent stem cells can also be generated and differentiated from somatic fish cells 422. Primordial germ cells can also be generated from somatic fish cells 423. Self-renewing fibroblasts can be selected such as through repeated passaging and selection of cell colonies that continue to proliferate 424. For example, adult differentiated fish fibroblasts can be serially passaged and grown, and selected for spontaneously transformed cells (e.g. spontaneously immortalized cells due to random mutation). Another method entails direct reprogramming of fibroblasts into myocytes 425. Another method entails immortalization of fish cells such as myosatellite cells or adipocyte precursor cells using various methods such as by viral transduction (e.g., TERT, SV40 Large T Antigen) 426. Pluripotent cells (e.g., embryonic stem cells 421, pluripotent stem cells 422, and primordial germ calls 423) can be grown to desired quantities 433 during the food production process. The pluripotent cells can then be differentiated into precursor cells such as pre-adipocytes 430 and/or myosatellite cells 429. In some cases, cells can be induced to differentiate into the desired cell type (e.g., muscle and/or fat cells) using genetic techniques and/or exogenous treatments 427. For example, pre-adipocytes (adipocyte precursors) 430 and myosatellite cells 429 can be induced to differentiate into adipocytes 432 and myocytes 431, respectively, using genetic manipulations such as gene editing and/or construct expression. Exogenous treatments can include small molecule treatment to induce differentiation. This can also include exposing cells to extracellular structures and/or signals such as microscaffolds optionally conjugated with differentiation/growth factors (e.g., FGF2). Finally, these processes can be assessed for scaling strategies 428 to enable large-scale production. Various combinations of the techniques described in FIGS. 4A-4B can be used for production of cultured food products. In an exemplary embodiment, adult differentiated cells such as fish (e.g., bass or salmon) fibroblasts are serially passaged to identify cells that have undergone spontaneous immortalization 424. These immortalized cells can be cultured to the desired quantity and then transdifferentiated 425 directly from the differentiated lineage into a desired lineage such as adipocytes and/or myocytes (or hepatocytes). Transdifferentiation can be accomplished using the various genetic modification techniques 427 such as using the expression constructs described herein.

Another approach not expressly shown in FIGS. 4A-4B utilizes mesenchymal stem cells (MSCs) for cultured food production. Mesenchymal stem cells are multipotent stromal cells capable of differentiating into various cell types including osteoblasts, chondrocytes, myocytes, and adipocytes. Mesenchymal stem cells can be derived from a variety of sources such as bone marrow and adipose tissue. For example, MSCs from the bone marrow can be isolated using flow cytometry or by plating directly on cell culture plates to form colony-forming unit fibroblasts. The MSCs can be grown in culture to desired quantities before they are induced to differentiate into a target differentiated cell type such as myocytes, adipocytes, hepatocytes, or any combination thereof. In some cases, the MSCs are split into separate cultures and differentiated separately before being combined to form a meat product (e.g., a mixture of myocytes and adipocytes, or hepatocytes and adipocytes). MSCs can be cultured using a variety of cell culture methods and can be grown using basal media supplemented with serum. In some cases, MSCs are cultured using a non-serum media. Sometimes, MSCs are cultured using a non-serum or low-serum media supplemented with a plant-based supplement such as mushroom-derived extract or soybean hydrolysate.

FIGS. 5A-5D show isolation and characterization of myosatellite cells isolated from trout. Where present, insets magnify image details, and the scale bar is equal to 10 μm in all micrographs. Substantially pure populations of piscine myosatellite cells are shown in FIG. 5A with the myosatellite cells making up about 80% of the isolated cells. FIG. 5B shows RT-PCR results confirming the presence of hallmark genes (Mstn1a, Myf5) expressed in these isolated myosatellite cells. FIG. 5C shows mature myocytes that were generated by differentiating myosatellite cells. The sheets of trout myotubes differentiated from the myosatellite cells are shown in FIG. 5D.

FIG. 6A shows co-cultures of salmon myosatellite cells (arrowheads) and salmon pre-adipocytes (arrows) (scale bar is 500 μm). FIG. 6B shows successful myocyte differentiation into a mature myocyte within the co-culture (scale bar is 10 μm).

FIG. 7A shows salmon fibroblasts that have been induced to form spheroids for propagation in a bioreactor (scale bar is 500 μm). FIG. 7B shows a spheroid that has been returned to 2-dimensional culture conditions to assess viability, which is confirmed by the cells from the spheroid migrating circumferentially to form colonies (scale bar is 500 μm).

FIG. 8 shows a culture of bass myosatellite cells that has been successfully cultivated according to the cell culture techniques disclosed herein.

FIG. 9 shows an embryonic stem cell colony derived from a duck egg. The ESCs formed colonies growing on a monolayer of mouse embryonic fibroblast (MEF) feeder cells as shown in FIG. 9.

FIG. 10A shows a population of duck hepatocytes in culture. These duck hepatocytes were assayed for markers of hepatocyte differentiation using reverse transcriptase polymerase chain reaction (RT-PCR). FIG. 10B shows the results of the RT-PCR assay comparing a control of undifferentiated cells (left lane) against the duck hepatocyte sample (right lane) for expression of the hepatocyte differentiation markers L-FABP, alpha-fetoprotein, and HNF3b. Beta actin was used as a control.

FIG. 11A shows self-renewing cells that were generated by culturing primary fibroblasts from duck and harvesting colonies of dividing cells after 6-8 weeks. FIG. 11B shows self-renewing cells that were generated by culturing primary fibroblasts from trout and harvesting colonies of dividing cells after 6-8 weeks. Both the duck and trout self-renewing cell colonies were characterized for morphology, proliferation rate, and proliferative capacity (number of passages achieved without changes in morphology, proliferative rate, and without genomic instability).

FIG. 12 shows an exemplary embodiment of a construct that can be introduced into a cell to provide inducible differentiation into a hepatocyte. The construct comprises a tetracycline responsive element (TRE) and ORFs for the hepatocyte reprogramming factors HNF1A, FOXA1, and HNF4A. The construct can be stably transformed into a target cell such as a pluripotent or multipotent cell. In some cases, the construct can be stably transformed into a terminally differentiated cell such as a fibroblast. The TRE suppresses expression of the ORFs but allows the ORFs to be transcribed in the presence of tetracycline or doxycycline. Thus, a cell line stably incorporating this construct can be induced to differentiate into a hepatocyte via treatment with tetracycline/doxycycline.

FIG. 13 shows an exemplary embodiment of a construct that can be introduced into a cell to allow inducible expression of one or more genes that predispose the cell to steatosis. The construct comprises a tetracycline responsive element (TRE) and the ORF for one or more genes involved in lipid metabolism such as ZFP423 (a zinc finger protein transcription factor) and/or ATF4 (activating transcription factor 4). The construct can be stably transformed into a target cell such as a pluripotent or multipotent cell. In some cases, the construct can be stably transformed into a terminally differentiated cell such as a fibroblast. The TRE suppresses expression of the ORFs but allows the ORFs to be transcribed in the presence of tetracycline or doxycycline. Thus, a cell line stably incorporating this construct can be induced to undergo steatosis or become predisposed to steatosis via treatment with tetracycline/doxycycline. In some cases, the construct comprises at least one, at least two, at least three, at least four, at least five, at least six, at least seven, at least eight, at least nine, at least ten, or at least eleven genes selected from the group consisting of: ATF4, ZFP423, LPIN1, PPAR, APOC3, APOE, ORL1, PEMT, MTTP, SREBP, STAT3, and KLF6.

FIG. 14 shows an exemplary embodiment of a DNA construct system that can be introduced into a cell to allow a proliferation/differentiation switch from a pluripotent phenotype into a differentiated phenotype. This system has a first construct comprising a pluripotency cassette providing constitutive expression of the ORFs for pluripotency factors (e.g. Oct4, Sox2, Klf4, I-Myc). The pluripotency factors of the first construct are flanked by pLox sites. The system has a second construct comprising a differentiation cassette providing tetracycline inducible expression of MyoD and Cre recombinase. The addition of an inducing agent such as tetracycline or doxycycline can induce expression of MyoD and Cre recombinase. MyoD expression can help cause the cell to undergo differentiation into a muscle cell. The Cre recombinase enzyme can catalyze the excision of the pluripotency factors flanked by the pLox sites. Then inducing agent can be removed to cease induction of MyoD and Cre recombinase expression. An advantage of this system is the low footprint left by the system following excision of the pluripotency factors and removal of the inducing agent.

FIG. 15 shows an exemplary construct that can be introduced into a cell to provide an inducible “off-switch”. This construct comprises one or more genes of interest and an expression cassette comprising TRE and Cre recombinase, which are flanked by pLox sites. Addition of an inducing agent can cause a cell line stably incorporating this construct to express Cre recombinase for catalyzing the excision of the intervening sequence flanked by the pLox sites. Thus, the one or more genes (e.g., one(s) that promote differentiation) and the TRE and Cre recombinase expression cassette are removed, resulting in footprint-free excision of the genes of interest.

FIG. 16A shows cultured duck hepatocytes. The top panel shows a negative control culture of duck hepatocytes untreated with linoleic acid. The close-up of the top panel shows the cell morphology of the hepatocytes. The bottom panel shows duck hepatocytes treated with 2 μM linoleic acid. The close-up of the bottom panel shows that the linoleic acid treated hepatocytes were successfully induced to undergo steatosis (an accumulation of lipid-containing vesicles—indicated by the arrowhead). FIG. 16B shows a dose response graph for linoleic acid treatment versus the percentage of hepatocytes having steatosis. As seen in the graph, increasing concentrations of linoleic acid (0, 0.1, 0.25, 0.5, 1, 2 μm) correlated with a corresponding increase in the percentage of hepatocytes with steatosis. At 2 μM linoleic acid, the percentage of steatotic hepatocytes was above 85%. Similar results were achieved with oleic acid and using alternative protocols with the following components: IBMX (a methyl xanthine), rosiglitazone (a thiazolidinedione), increased glucose concentration, other fatty acid species, and/or corticosteroids such as dexamethasone.

FIG. 17 shows a graph plotting the number of cells from an immortalized cell line derived from adult duck hepatocytes by selecting rapidly proliferating hepatocytes following serial passaging. These immortalized cells were cultured in progressively decreasing concentrations of fetal bovine serum (FBS) in the presence of soybean hydrolysate (10 g/L). The number of hepatocytes and the percentage of FBS are graphed over time with the hepatocytes starting at below 4 million cells and 10% serum and gradually increasing in number until just above ten million cells at below 2% serum (0.8%) over a period of 20 days. The media supplementation of soybean hydrolysate allowed the serum requirements of the cultured cells to be reduced by 92%.

FIG. 18 shows duck fibroblasts that have also been successfully grown in 10% shiitake mushroom extract after successive reduction of fetal bovine serum from the cell culture media.

FIG. 19A shows duck fibroblasts grown in serum-free media without additional supplementation; FIG. 19B shows a control culture grown in DMEM supplemented with 10% fetal bovine serum.

FIG. 20 shows one embodiment of a bioreactor system used for cell culture. The bioreactor system comprises a reactor chamber 2001 for culturing cells and a stirring element 2003 for agitating the contents of the reactor chamber 2001. Media is added into the reactor chamber via at least one input port 2002. The media is sometimes maintenance media, differentiation media, steatotic media, proliferation media, or any other media formulation disclosed herein. Media is removed from the reactor chamber via at least one output port 2007. In some cases, oxygen, carbon dioxide, and/or other gases are introduced through at least one input gas port 2006. The input gas port 2006 is optionally coupled to an aerator positioned inside the reactor chamber. Oftentimes, the bioreactor system comprises at least one sensor 2004 for monitoring the reactor chamber. The at least one sensor 2004 is usually in communication with a control unit 2008 (e.g. a computer). In many cases, the reactor chamber is seeded with a plurality of micro-scaffolds 2005. The micro-scaffolds 2005 enable adherence of certain adherent cells such as, for example, hepatocytes.

FIG. 21 shows an illustrative process by which a bioreactor system is used for meat production. Specialized cells are isolated from an egg and grown in the bioreactor. The cells are grown using media comprising water and nutrients created from plants (e.g. as a substitute for serum). The cells are grown in the sterile environment of the bioreactor for 4-6 weeks. Finally, the cells are harvested and/or processed into a meat product.

FIG. 22A and FIG. 22B show a spheroid formed from duck hepatocytes growing in a hanging drop and a spinner flask into which the spheroid can be transferred for 3-dimensional suspension culture. As shown in FIG. 22A, cells are grown in “hanging drops” of media and develop into spheroids and are then transferred to spinner flasks and grown in 3-dimensional suspension culture to allow scaling up of cell production.

FIG. 23A shows fish myosatellite cells grown on glucomannan microscaffolds (10% w/v) that have differentiated to form 3-dimensional myotubes. FIG. 23B shows a negative control of undifferentiated myosatellite cells from the same preparation grown in identical cell culture conditions.

FIG. 24A shows duck fibroblasts (arrowheads) successfully grown on glucomannan microscaffolds (arrows). FIG. 24B shows a representative glucomannan microscaffold.

FIG. 25 shows duck muscle tissue that was created by differentiation of myosatellite cells. This figure represents a still photo from a movie demonstrating spontaneous myocyte contraction.

FIG. 26 shows additional exemplary food products for human consumption generated according to the methods disclosed herein. The left panel shows a duck liver pâté made using duck steatotic liver cells. The right panel shows a fois gras butter made using duck steatotic liver cells.

FIG. 27 shows exemplary food products for human consumption generated according to the methods disclosed herein. The left panel shows a salmon pâté. The right panel shows a duck meat pâté. In addition, chicken meat pâtés have also been developed.

FIG. 28A shows an exemplary embodiment of a method of Cre delivery for the purpose of activating/silencing particular genes. FIG. 28B shows different methods of using Cre to induce a “switch” between activated gene sets relevant to meat creation (e.g., proliferation and differentiation).

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, “hepatocyte” refers to liver cells, hepatocytes, and hepatocyte-like cells. Hepatocytes are of animal origin and can be derived from various avian species including, as non-limiting examples, duck (e.g. Mulard duck, Barbary duck), goose (e.g. grey Landes goose), chicken, turkey, emu, Cornish chicken, Japanese quail, Plymouth Rock chicken, and ostrich.

As used herein, “high lipid accumulation” refers to the formation of lipid-containing vacuoles or vesicles inside of at least 10%, 20%, 30%, 40%, 50%, 60%, 70%, 80%, 90%, 95%, or 99% of the cells in a population of cells. In some cases, “high lipid accumulation” refers to the formation of lipid-containing vacuoles or vesicles inside of a majority (e.g. greater than half) of the cells in a population of cells.

As used herein, “self-renewal” or “self-renewing” refers to cell division or proliferation while maintaining a certain cell type (e.g. an undifferentiated state). Examples of self-renewing cells include embryonic stem cells, induced pluripotent stem cells, and multipotent stem cells (e.g. myosatellite cells, hepatoblasts, and other progenitor cells). In some cases, self-renewing cells include differentiated cells that have been immortalized (e.g. via spontaneous immortalization).

As used herein, “about” refers to a range of 10% around a particular quantity, unless stated otherwise. For example, about 10 liters (L) refers to 9 to 11 L.

In all cases where the term “about” is used in relation to a number or range, it is contemplated in some cases to mean about, or to optionally replace “about” with “exactly.”

### NUMBERED EMBODIMENTS

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A method of producing cultured cells having high lipid accumulation for human consumption, the method comprising: culturing a population of cells; inducing differentiation within the population of cells; inducing high lipid accumulation within the population of cells; and processing the population of cells for human consumption. 2. The method of embodiment 1, wherein the population of cells following differentiation comprises hepatocytes. 3. The method of embodiment 1, wherein processing comprises preparing the population of cells as foie gras. 4. The method of embodiment 1, wherein the population of cells is derived from duck or goose. 5. The method of embodiment 1, wherein the population of cells is derived from at least one of poultry and livestock. 6. The method of embodiment 1, wherein inducing high lipid accumulation comprises inducing steatosis. 7. The method of embodiment 1, wherein high lipid accumulation is characterized by excess accumulation of cytoplasmic lipid droplets. 8. The method of embodiment 1, wherein inducing high lipid accumulation comprises exposing the population of cells to an exogenous compounds that modulates at least one lipid metabolic pathway. 9. The method of embodiment 1, wherein inducing high lipid accumulation comprises exposing the population of cells to at least one of a toxin and a high lipid concentration. 10. The method of embodiment 1, wherein inducing high lipid accumulation comprises modulating at least one lipid metabolic pathway to enhance lipid retention within the population of cells. 11. The method of embodiment 1, wherein the population of cells is modified to express at least one gene for inducing differentiation into hepatocytes upon treatment with an induction agent. 12. The method of embodiment 11, wherein the at least one gene for inducing differentiation into hepatocytes comprises at least one of Hepatocyte Nuclear Factor 1 Alpha (HNF1A), Forkhead Box A2 (FOXA2), and Hepatocyte Nuclear Factor 4 Alpha (HNF4A). 13. The method of embodiment 1, wherein inducing high lipid accumulation comprises modifying the population of cells to generate a modified cell line configured to express at least one gene for enhancing steatosis. 14. The method of embodiment 13, wherein the modified cell line is configured to express at least one gene for enhancing steatosis upon treatment with an induction agent. 15. The method of embodiment 13, wherein the modified cell line is stably transformed with a construct comprising an open reading frame (ORF) encoding ATF4, ZFP423, LPIN1, PPAR, APOC3, APOE, ORL1, PEMT, MTTP, SREBP, STAT3, or KLF6. 16. The method of embodiment 13, wherein the modified cell line is stably transformed with a construct that facilitates expression of the at least one gene for enhancing steatosis when the modified cell line is exposed to tetracycline or a derivative thereof 17. The method of embodiment 1, wherein inducing high lipid accumulation comprises altering at least one gene in at least one cell within the population of cells to modulate lipid metabolism. 18. The method of embodiment 1, wherein the population of cells comprises liver, heart, kidney, stomach, intestine, lung, diaphragm, esophagus, thymus, pancreas, or tongue cells after differentiation. 19. The method of embodiment 1, wherein processing the population of cells for human consumption comprises blending the population of cells with cells having low lipid accumulation. 20. The method of embodiment 1, wherein the population of cells is isolated as embryonic stem cells. 21. The method of embodiment 1, wherein the population of cells has been modified to induce pluripotency. 22. The method of embodiment 1, wherein the population of cells is isolated as multipotent adult stem cells. 23. The method of embodiment 1, wherein culturing comprises growing and expanding the population of cells in cell culture. 24. The method of embodiment 1, wherein inducing differentiation comprises exposing the population of cells to culture conditions that stimulate differentiation. 25. The method of embodiment 1, wherein inducing differentiation comprises exposing the population of cells to at least one growth factor that stimulates differentiation. 26. The method of embodiment 1, wherein culturing comprises growing the population of cells on a two dimensional surface. 27. The method of embodiment 1, wherein culturing comprises growing the population of cells on a three-dimensional scaffold. 28. The method of embodiment 1, wherein culturing comprises growing the population of cells on micro-scaffolds within a bioreactor, wherein the micro-scaffolds enable cell adhesion. 29. The method of embodiment 28, wherein the micro-scaffolds comprise glucomannan or alginate. 30. The method of embodiment 1, wherein the population of cells does not require an adherence substrate for survival and proliferation. 31. The method of embodiment 1, wherein the population of cells is adapted to suspension culture. 32. The method of embodiment 1, wherein the population of cells forms non-textured tissue after differentiation. 33. The method of embodiment 1, wherein the population of cells forms non-muscle tissue after differentiation. 34. The method of embodiment 1, wherein culturing comprises growing the population of cells in a media formulation comprising at least one nutritional supplement. 35. The method of embodiment 34, wherein the at least one nutritional supplement comprises an omega-3 fatty acid. 36. The method of embodiment 34, wherein the at least one nutritional supplement comprises a polyunsaturated fatty acid. 37. The method of embodiment 34, wherein the at least one nutritional supplement comprises a monounsaturated fatty acid. 38. The method of embodiment 34, wherein the at least one nutritional supplement comprises linoleic acid, oleic acid, or a combination thereof. 39. The method of embodiment 1, wherein the population of cells is cultured using a non-serum media formulation. 40. The method of embodiment 1, wherein the population of cells is cultured using a mushroom-based media formulation. 41. The method of embodiment 1, wherein the population of cells is cultured using a media formulation comprising soybean hydrolysate. 42. A method of producing cultured non-textured tissue having high lipid content, the method comprising: obtaining a population of differentiated cells capable of self-renewal; culturing the population of differentiated cells; manipulating at least one lipid metabolic pathway to induce steatosis in the population of differentiated cells such that the cells accumulate high lipid content; and processing the population of differentiated cells into non-textured tissue. 43. The method of embodiment 42, wherein obtaining the population of differentiated cells capable of self-renewal comprises transforming differentiated cells into immortalized cells. 44. The method of embodiment 42, wherein obtaining the population of differentiated cells capable of self-renewal comprises culturing differentiated cells until spontaneous mutations give rise to immortalized cells. 45. The method of embodiment 42, wherein the population of differentiated cells comprises hepatocytes. 46. The method of embodiment 42, wherein processing comprises using the population of differentiated cells as an ingredient in foie gras. 47. The method of embodiment 42, wherein the population of differentiated cells is derived from duck or goose. 48. The method of embodiment 42, wherein the population of differentiated cells is derived from at least one of poultry and livestock. 49. The method of embodiment 42, wherein steatosis is characterized by excess accumulation of cytoplasmic lipid droplets. 50. The method of embodiment 42, wherein manipulating the at least one lipid metabolic pathway comprises exposing the population of cells to an exogenous compound. 51. The method of embodiment 42, wherein manipulating the at least one lipid metabolic pathway comprises exposing the population of differentiated cells to at least one of a toxin and a high lipid concentration. 52. The method of embodiment 42, wherein manipulating the at least one lipid metabolic pathway comprises altering at least one gene in within the population of differentiated cells to modulate lipid metabolism. 53. The method of embodiment 42, wherein manipulating the at least one lipid metabolic pathway comprises modifying the population of differentiated cells with a genetic construct to generate a modified cell line configured to express at least one gene for enhancing steatosis. 54. The method of embodiment 53, wherein the modified cell line is configured to express at least one gene for enhancing steatosis upon treatment with an induction agent. 55. The method of embodiment 53, wherein the modified cell line is stably transformed with a construct comprising an open reading frame (ORF) encoding ATF4, ZFP423, LPIN1, PPAR, APOC3, APOE, ORL1, PEMT, MTTP, SREBP, STAT3, or KLF6. 56. The method of embodiment 53, wherein the modified cell line is stably transformed with a construct that facilitates expression of the at least one gene for enhancing steatosis when the modified cell line is exposed to tetracycline or a derivative thereof. 57. The method of embodiment 42, wherein the population of differentiated cells comprises liver, heart, kidney, stomach, intestine, lung, diaphragm, esophagus, thymus, pancreas, or tongue cells. 58. The method of embodiment 42, wherein processing the population of differentiated cells comprises blending the population of cells with cells having low lipid accumulation. 59. The method of embodiment 42, wherein culturing comprises growing and expanding the population of cells in cell culture. 60. The method of embodiment 42, wherein culturing comprises growing the population of cells on a two dimensional surface. 61. The method of embodiment 42, wherein culturing comprises growing the population of cells on a three-dimensional scaffold. 62. The method of embodiment 42, wherein culturing comprises growing the population of cells on micro-scaffolds within a bioreactor, wherein the micro-scaffolds enable cell adhesion. 63. The method of embodiment 62, wherein the micro-scaffolds comprise glucomannan or alginate. 64. The method of embodiment 42, wherein the population of cells does not require an adherence substrate for survival and proliferation. 65. The method of embodiment 42, wherein the population of cells is adapted to suspension culture. 66. The method of embodiment 42, wherein the population of differentiated cells form non-textured tissue. 67. The method of embodiment 42, wherein the population of cells forms non-muscle tissue. 68. The method of embodiment 42, wherein culturing comprises growing the population of cells in a media formulation comprising at least one nutritional supplement. 69. The method of embodiment 68, wherein the at least one nutritional supplement comprises an omega-3 fatty acid. 70. The method of embodiment 68, wherein the at least one nutritional supplement comprises a polyunsaturated fatty acid. 71. The method of embodiment 68, wherein the at least one nutritional supplement comprises a monounsaturated fatty acid. 72. The method of embodiment 68, wherein the at least one nutritional supplement comprises linoleic acid, oleic acid, or a combination thereof. 73. The method of embodiment 42, wherein the population of cells is cultured using a non-serum media formulation. 74. The method of embodiment 42, wherein the population of cells is cultured using a mushroom-based media formulation. 75. The method of embodiment 42, wherein the population of cells is cultured using a media formulation comprising soybean hydrolysate. 76. A method of producing cultured non-muscle tissue for human consumption, the method comprising: obtaining a population of self-renewing cells; culturing the population of self-renewing cells; inducing differentiation in the population of cells to form non-muscle tissue; and processing the cultured non-muscle tissue for human consumption. 77. A method of producing cultured tissue for human consumption, the method comprising: obtaining a population of self-renewing cells; adapting the population of self-renewing cells to suspension culture; culturing the population of self-renewing cells; inducing differentiation in the population of cells to form cultured tissue; and processing the cultured tissue for human consumption. 78. A method of producing cultured non-textured muscle tissue for human consumption, the method comprising: obtaining a population of self-renewing cells; culturing the population of self-renewing cells; inducing differentiation in the population of cells to form non-textured muscle tissue; and processing the cultured non-textured muscle tissue for human consumption. 79. The method of embodiment 78, wherein the non-textured muscle tissue is octopus, squid, or cuttlefish muscle. 80. The method of embodiment 78, wherein inducing differentiation in the population of cells comprises generating myotubes. 81. The method of embodiment 80, wherein the population of cells following differentiation comprises myotubes that are at least 50 μm in length. 82. The method of embodiment 78, wherein the non-textured muscle tissue is fish muscle tissue. 83. The method of embodiment 82, wherein the fish muscle tissue comprises high glycolytic and anaerobic muscle fibers. 84. The method of embodiment 83, wherein the high glycolytic and anaerobic muscle fibers make up at least 80% of the fish muscle tissue. 85. The method of embodiment 82, wherein the population of cells is derived from sea bass, tuna, mackerel, blue marlin, swordfish, yellowtail, salmon, or trout. 86. The method of embodiment 82, wherein the non-textured muscle tissue is combined with fat tissue. 87. The method of embodiment 86, wherein the fish muscle and fat tissue is sushi-grade. 88. The method of embodiment 78, wherein the population of cells is isolated as embryonic stem cells. 89. The method of embodiment 78, wherein the population of cells has been modified to induce pluripotency. 90. The method of embodiment 78, wherein the population of cells has been modified to incorporate a genetic construct comprising an open reading frame (ORF) of at least one gene configured to induce differentiation in the population of cells into myocytes. 91. The method of embodiment 90, wherein the at least one gene configured to induce differentiation comprises Myogenin (MyoG), Myogenic Differentiation 1 (MyoD), Myogenic Factor 6 (MRF4), Myogenic Factor 5 (MYF5), or any combination thereof. 92. The method of embodiment 89, wherein the population of cells has been modified to incorporate: a first genetic construct comprising an open reading frame (ORF) of at least one pluripotency gene configured to promote cell division; and a second genetic construct comprising an open reading frame (ORF) of a regulatory factor configured to inactivate the at least one pluripotency gene. 93. The method of embodiment 92, wherein the at least one pluripotency gene is configured to promote at least 50 cell divisions. 94. The method of embodiment 92, wherein the regulatory factor is a recombinase, and the open reading frame (ORF) of at least one pluripotency gene is flanked by recombination sequences recognized by the recombinase such that expression of the recombinase catalyzes excision of the open reading frame (ORF) of at least one pluripotency gene. 95. The method of embodiment 92, wherein the second genetic construct comprises an open reading frame (ORF) of at least one cell lineage gene for differentiating the cell line and an inducible promoter controlling expression of the open reading frame (ORF) of at least one cell lineage gene and the open reading frame (ORF) of the regulatory factor. 96. The method of embodiment 95, wherein inducing differentiation comprises exposing the population of cells to an induction agent to induce expression of the open reading frame (ORF) of at least one cell lineage gene and the open reading frame (ORF) of the regulatory factor. 97. The method of embodiment 96, further comprising removing the induction agent after exposing the population of cells to the induction agent and before the processing the cultured non-textured muscle tissue for human consumption. 98. The method of embodiment 78, wherein the population of cells is isolated as multipotent adult stem cells. 99. The method of embodiment 78, wherein culturing comprises growing and expanding the population of cells in cell culture. 100. The method of embodiment 78, wherein inducing differentiation comprises exposing the population of cells to culture conditions that stimulate differentiation. 101. The method of embodiment 78, wherein inducing differentiation comprises exposing the population of cells to at least one growth factor that stimulates differentiation. 102. The method of embodiment 78, wherein culturing comprises growing the population of cells on a two dimensional surface. 103. The method of embodiment 78, wherein culturing comprises growing the population of cells on a three-dimensional scaffold. 104. The method of embodiment 78, wherein culturing comprises growing the population of cells on micro-scaffolds within a bioreactor, wherein the micro-scaffolds enable cell adhesion. 105. The method of embodiment 104, wherein the micro-scaffolds comprise glucomannan or alginate. 106. The method of embodiment 78, wherein the population of cells does not require an adherence substrate for survival and proliferation. 107. The method of embodiment 78, wherein the population of cells is adapted to suspension culture. 108. The method of embodiment 78, wherein the population of cells forms non-textured tissue after differentiation. 109. The method of embodiment 78, wherein the population of cells forms non-muscle tissue after differentiation. 110. The method of embodiment 78, wherein culturing comprises growing the population of cells in a media formulation comprising at least one nutritional supplement. 111. The method of embodiment 110, wherein the at least one nutritional supplement comprises an omega-3 fatty acid. 112. The method of embodiment 110, wherein the at least one nutritional supplement comprises a polyunsaturated fatty acid. 113. The method of 110, wherein the at least one nutritional supplement comprises a monounsaturated fatty acid. 114. The method of embodiment 110, wherein the at least one nutritional supplement comprises linoleic acid, oleic acid, or a combination thereof 115. The method of embodiment 78, wherein the population of cells is cultured using a non-serum media formulation. 116. The method of embodiment 78, wherein the population of cells is cultured using a mushroom-based media formulation. 117. The method of embodiment 78, wherein the population of cells is cultured using a media formulation comprising soybean hydrolysate. 118. A method of preparing foie gras comprising cultured avian liver tissue, the method comprising: obtaining a population of avian derived cells capable of self-renewal; differentiating the population of avian derived cells into hepatocytes; and inducing steatosis in the hepatocytes to generate cultured avian liver tissue having high lipid content; and preparing the cultured avian liver tissue as foie gras. 119. The method of embodiment 118, wherein the avian derived cells are duck cells. 120. The method of embodiment 118, wherein the avian derived cells are goose cells. 121. A culinary foie gras composition comprising tissue cultured hepatocytes having high lipid content and processed for human consumption. 122. The composition of embodiment 121, wherein the composition has been processed into a plurality of slices. 123. The composition of embodiment 122, wherein each slice weighs no more than about 5 ounces. 124. The composition of embodiment 122, wherein each slice is individually packaged. 125. The composition of embodiment 121, wherein the foie gras composition weighs at least about 1.5 pounds, is round and firm, and has no blemish. 126. The composition of embodiment 121, wherein the foie gras composition has a package label indicating an A grade rating for the foie gras composition. 127. The composition of embodiment 121, wherein the foie gras composition weighs between about 0.75 to about 1.5 pounds. 128. The composition of embodiment 121, wherein the foie gras composition has a package label indicating a B grade rating for the foie gras composition. 129. The composition of embodiment 121, wherein the foie gras composition weighs less than about 1 pound and has no more than three blemishes. 130. The composition of embodiment 121, wherein the foie gras composition has a package label indicating a C grade rating for the foie gras composition. 131. The composition of embodiment 121, wherein the tissue cultured hepatocytes are steatotic. 132. The composition of embodiment 121, wherein the tissue cultured hepatocytes are characterized by excess accumulation of cytoplasmic lipid droplets. 133. The composition of embodiment 121, wherein the high lipid content is obtained by exposure to an exogenous compound that modulates at least one lipid metabolic pathway. 134. The composition of embodiment 121, wherein the high lipid content is obtained by exposure to at least one of a toxin and a high lipid concentration. 135. The composition of embodiment 121, wherein the high lipid content is obtained by modulation of at least one lipid metabolic pathway to enhance lipid retention within the population of cells. 136. The composition of embodiment 121, wherein the high lipid content is obtained by alteration of at least one gene in the tissue cultured hepatocytes. 137. The composition of embodiment 121, wherein the foie gras composition further comprises cells having low lipid accumulation. 138. The composition of embodiment 121, wherein the tissue cultured hepatocytes are differentiated from isolated embryonic stem cells. 139. The composition of embodiment 121, wherein the tissue cultured hepatocytes are differentiated from induced pluripotent stem cells. 140. The composition of embodiment 121, wherein the tissue cultured hepatocytes are differentiated from isolated multipotent adult stem cells. 141. The composition of embodiment 121, wherein the tissue cultured hepatocytes are generated by differentiation in a population of cells capable of self-renewal. 142. The composition of embodiment 141, wherein differentiation comprises exposing the population of cells to culture conditions that stimulate differentiation. 143. The composition of embodiment 141, wherein differentiation comprises exposing the population of cells to at least one growth factor that stimulates differentiation. 144. The composition of embodiment 121, wherein the tissue cultured hepatocytes are grown on a two dimensional surface. 145. The composition of embodiment 121, wherein the tissue cultured hepatocytes are grown on a three-dimensional scaffold. 146. The composition of embodiment 121, wherein the tissue cultured hepatocytes are grown on micro-scaffolds within a bioreactor, wherein the micro-scaffolds enable cell adhesion. 147. The composition of embodiment 121, wherein the tissue cultured hepatocytes do not require an adherence substrate for survival and proliferation. 148. The composition of embodiment 121, wherein the tissue cultured hepatocytes are adapted to suspension culture. 149. The composition of embodiment 121, wherein the tissue cultured hepatocytes form non-textured tissue. 150. The composition of embodiment 121, wherein the tissue cultured hepatocytes form non-muscle tissue. 151. The composition of embodiment 121, wherein the tissue cultured hepatocytes are cultured in a media formulation comprising at least one nutritional supplement. 152. The composition of embodiment 151, wherein the at least one nutritional supplement comprises an omega-3 fatty acid. 153. The composition of embodiment 151, wherein the at least one nutritional supplement comprises a polyunsaturated fatty acid. 154. The composition of 151, wherein the at least one nutritional supplement comprises a monounsaturated fatty acid. 155. A composition comprising cultured organ cells processed into a non-textured non-muscle food product for human ingestion. 156. The composition of embodiment 155, wherein the cultured organ cells comprise hepatocytes. 157. The composition of embodiment 155, wherein the cultured organ cells comprise avian cells. 158. The composition of embodiment 155, wherein the food product is processed into a plurality of slices. 159. The composition of embodiment 158, wherein each slice weighs no more than about 5 ounces. 160. The composition of embodiment 158, wherein each slice is individually packaged. 161. The composition of embodiment 155, wherein the food product is foie gras. 162. The composition of embodiment 161, wherein the foie gras weighs at least about 1.5 pounds, is round and firm, and has no blemish. 163. The composition of embodiment 161, wherein the foie gras has a package label indicating an A grade rating. 164. The composition of embodiment 161, wherein the foie gras weighs between about 0.75 to about 1.5 pounds. 165. The composition of embodiment 161, wherein the foie gras has a package label indicating a B grade rating. 166. The composition of embodiment 161, wherein the foie gras weighs less than about 1 pound and has no more than three blemishes. 167. The composition of embodiment 161, wherein the foie gras has a package label indicating a C grade rating. 168. The composition of embodiment 161, wherein the tissue cultured hepatocytes are steatotic. 169. The composition of embodiment 161, wherein the foie gras is characterized by high lipid content. 170. The composition of embodiment 169, wherein the high lipid content is obtained by exposure to an exogenous compound that modulates at least one lipid metabolic pathway. 171. The composition of embodiment 169, wherein the high lipid content is obtained by exposure to at least one of a toxin and a high lipid concentration. 172. The composition of embodiment 169, wherein the high lipid content is obtained by modulation of at least one lipid metabolic pathway to enhance lipid retention within the population of cells. 173. The composition of embodiment 169, wherein the high lipid content is obtained by alteration of at least one gene in the tissue cultured hepatocytes. 174. The composition of embodiment 169, wherein the foie gras composition further comprises cells having low lipid accumulation. 175. The composition of embodiment 155, wherein the cultured organ cells are grown on a two dimensional surface. 176. The composition of embodiment 155, wherein the cultured organ cells are grown on a three-dimensional scaffold. 177. The composition of embodiment 155, wherein the cultured organ cells are grown on micro-scaffolds within a bioreactor, wherein the micro-scaffolds enable cell adhesion. 178. The composition of embodiment 155, wherein the cultured organ cells do not require an adherence substrate for survival and proliferation. 179. The composition of embodiment 155, wherein the cultured organ cells are adapted to suspension culture. 180. The composition of embodiment 155, wherein the cultured organ cells form non-textured tissue. 181. The composition of embodiment 155, wherein the cultured organ cells form non-muscle tissue. 182. The composition of embodiment 155, wherein the cultured organ cells are cultured in a media formulation comprising at least one nutritional supplement. 183. The composition of embodiment 182, wherein the at least one nutritional supplement comprises an omega-3 fatty acid. 184. The composition of embodiment 182, wherein the at least one nutritional supplement comprises a polyunsaturated fatty acid. 185. The composition of embodiment 182, wherein the at least one nutritional supplement comprises a monounsaturated fatty acid. 186. The composition of embodiment 182, wherein the cultured organ cells are cultured using a non-serum media formulation. 187. The composition of embodiment 182, wherein the cultured organ cells are cultured using a mushroom-based media formulation. 188. An edible foie gras composition comprising cultured steatotic avian liver cells and seasoning. 189. The composition of embodiment 188, wherein the seasoning includes at least one of salt, pepper, and sugar. 190. A foie gras composition comprising cultured liver cells having high lipid content and liver cells having low lipid content. 191. The composition of embodiment 190, wherein the cultured liver cells having high lipid content and the liver cells having low lipid content are blended together. 192. The composition of embodiment 190, wherein the foie gras composition is suitable as an ingredient for preparing one of a mousse, a parfait, and a pâté. 193. The composition of embodiment 190, wherein the liver cells having low lipid content are cultured cells. 194. The composition of embodiment 190, wherein the liver cells having low lipid content are un-cultured cells. 195. An edible composition comprising avian liver cells grown in cell culture and processed for human consumption. 196. A packaged foie gras composition comprising cultured liver cells and packaging having a label indicating the foie gras composition was not produced by forced feeding. 197. A packaged foie gras composition comprising cultured liver cells processed into foie gras and packaging having a label indicating the foie gras was produced in a pathogen-free environment. 198. The composition of embodiment 197, wherein the label indicates the composition was produced without exposure to avian bird flu virus. 199. A packaged edible composition comprising cultured cells processed into a food product and packaging having a label indicating the composition was produced without exposure to a toxin. 200. The composition of embodiment 199, wherein the toxin is one of an insecticide, herbicide, and fungicide. 201. A method of producing cultured cells for human consumption without using antibiotics, the method comprising: culturing a population of cells without using antibiotics; inducing differentiation within the population of cells; inducing high lipid accumulation within the population of cells; and processing the population of cells for human consumption. 202. A method of producing cultured cells for human consumption without exposure to pathogens, the method comprising: culturing a population of cells in a pathogen-free culture environment; inducing differentiation within the population of cells; inducing high lipid accumulation within the population of cells; and processing the population of cells for human consumption. 203. A method of producing cultured cells for human consumption without exposure to toxins, the method comprising: culturing a population of cells in a toxin-free culture environment; inducing differentiation within the population of cells; inducing high lipid accumulation within the population of cells; and processing the population of cells for human consumption. 204. A method of producing cultured non-textured tissue having high lipid content and no vasculature, the method comprising: culturing a population of cells; inducing differentiation in the population of cells; manipulating lipid metabolic pathways to induce steatosis in the population of cells such that the cells accumulate high lipid content; and processing the population of cells into non-textured tissue having no vasculature. 205. A method of producing cultured tissue having increased nutritional content for human consumption, the method comprising: culturing a population of cells in a culture medium having at least one nutritional supplement; manipulating lipid metabolic pathways to induce steatosis in the population of differentiated cells such that the cells accumulate high lipid content; and processing the population of differentiated cells into non-textured tissue having no vasculature for human consumption. 206. The method of embodiment 205, wherein the at least one nutritional supplement comprises an omega-3 fatty acid. 207. The method of embodiment 205, wherein the at least one nutritional supplement comprises a polyunsaturated fatty acid. 208. The method of embodiment 205, wherein the at least one nutritional supplement comprises a monounsaturated fatty acid. 209. A method of producing cultured organ tissue for human consumption, the method comprising: culturing a population of cells capable of self-renewal; inducing differentiation in the population of cells to generate organ tissue; and processing the organ tissue for human consumption. 210. The method of embodiment 209, wherein the organ tissue is liver, heart, kidney, stomach, intestine, lung, diaphragm, esophagus, thymus, pancreas, or tongue tissue. 211. The method of embodiment 210, wherein the organ tissue is liver tissue. 212. The method of embodiment 211, wherein processing comprises blending the organ tissue with additional cellular tissues. 213. The method of embodiment 212, wherein the additional cellular tissues comprise non-steatotic liver cells. 214. A method of producing cultured fish tissue having enhanced nutritional content for human consumption, the method comprising: culturing a population of fish myocytes in a culture media having at least one nutritional supplement; expanding the population of myocytes; and processing the population of myocytes into fish tissue for human consumption. 215. The method of embodiment 214, wherein the fish tissue comprises fast twitch muscle fibers. 216. The method of embodiment 214, further comprising combining the population of myocytes with a population of adipocytes. 217. The method of embodiment 214, wherein the fish myocytes are salmon myocytes. 218. The method of embodiment 214, wherein the fish myocytes are tuna myocytes. 219. The method of embodiment 214, wherein the fish myocytes are trout myocytes. 220. An edible composition comprising fish tissue produced from cultured myocytes and adipocytes. 221. A method of producing cultured fish meat for human consumption, the method comprising: obtaining a population of self-renewing cells derived from fish; culturing the population of self-renewing cells in culture media comprising micro-scaffolds; inducing differentiation in the population of cells to form at least one of myocytes and adipocytes; and processing the population of cells into fish meat for human consumption. 222. The method of embodiment 221, wherein the micro-scaffolds comprise glucomannan or alginate. 223. The method of embodiment 221, wherein at least a subset of the population of cells has been modified to incorporate a genetic construct comprising an open reading frame (ORF) of at least one gene configured to induce differentiation in the population of cells into myocytes. 224. The method of embodiment 223, wherein the at least one gene configured to induce differentiation comprises Myogenin (MyoG), Myogenic Differentiation 1 (MyoD), Myogenic Factor 6 (MRF4), Myogenic Factor 5 (MYF5), or any combination thereof. 225. The method of embodiment 221, wherein at least a subset of the population of cells has been modified to incorporate a genetic construct comprising an open reading frame (ORF) of at least one gene configured to induce differentiation in the population of cells into adipocytes. 226. The method of embodiment 221, wherein at least a subset of the population of cells has been modified to incorporate: a first genetic construct comprising an open reading frame (ORF) of at least one pluripotency gene configured to promote cell division; and a second genetic construct comprising an open reading frame (ORF) of a regulatory factor configured to inactivate the at least one pluripotency gene. 227. The method of embodiment 226, wherein the regulatory factor is a recombinase, and the open reading frame (ORF) of at least one pluripotency gene is flanked by recombination sequences recognized by the recombinase such that expression of the recombinase catalyzes excision of the open reading frame (ORF) of at least one pluripotency gene. 228. The method of embodiment 226, wherein the second genetic construct comprises an open reading frame (ORF) of at least one cell lineage gene for differentiating the cell line and an inducible promoter controlling expression of the open reading frame (ORF) of at least one cell lineage gene and the open reading frame (ORF) of the regulatory factor. 229. The method of embodiment 228, wherein inducing differentiation comprises exposing the population of cells to an induction agent to induce expression of the open reading frame (ORF) of at least one cell lineage gene and the open reading frame (ORF) of the regulatory factor. 230. The method of embodiment 229, further comprising removing the induction agent after exposing the population of cells to the induction agent and before the processing the population of cells into fish meat for human consumption. 231. The method of embodiment 221, wherein the fish meat is sushi. 232. The method of embodiment 221, wherein the fish meat is surimi. 233. The method of embodiment 221, wherein the fish meat is suitable for raw consumption. 234. The method of embodiment 221, wherein the fish meat is cooked. 235. The method of embodiment 221, wherein the fish meat is salmon meat. 236. The method of embodiment 221, wherein the fish meat is sushi-grade salmon meat. 237. The method of embodiment 221, wherein the fish meat is tuna meat. 238. The method of embodiment 221, wherein the fish meat is sushi-grade tuna meat. 239. The method of embodiment 221, wherein the fish meat is trout meat. 240. The method of embodiment 221, wherein inducing differentiation in (c) causes the population of cells to form myocytes and adipocytes. 241. The method of embodiment 240, wherein the fish meat is composed of at least 50% high glycolytic and anaerobic muscle fibers. 242. The method of embodiment 221, wherein the population of cells is derived from sea bass, tuna, mackerel, blue marlin, swordfish, yellowtail, salmon, or trout. 243. The method of embodiment 221, wherein processing in (d) comprises combining the population of cells with a second population of cells composed of myocytes or adipocytes. 244. The method of embodiment 221, wherein the population of cells is isolated as embryonic stem cells. 245. The method of embodiment 221, wherein the population of cells has been modified to induce pluripotency. 246. The method of embodiment 221, wherein the population of cells is isolated as multipotent adult stem cells. 247. The method of embodiment 221, wherein culturing comprises growing and expanding the population of cells in cell culture. 248. The method of embodiment 221, wherein inducing differentiation comprises exposing the population of cells to culture conditions that stimulate differentiation. 249. The method of embodiment 221, wherein inducing differentiation comprises exposing the population of cells to at least one growth factor that stimulates differentiation. 250. The method of embodiment 221, wherein culturing comprises growing the population of cells on a two dimensional surface. 251. The method of embodiment 221, wherein culturing comprises growing the population of cells on a three-dimensional scaffold. 252. The method of embodiment 221, wherein culturing comprises growing the population of cells on micro-scaffolds within a bioreactor, wherein the micro-scaffolds enable cell adhesion. 253. The method of embodiment 221, wherein the population of cells forms non-textured tissue after differentiation. 254. The method of embodiment 221, wherein culturing comprises growing the population of cells in a media formulation comprising at least one nutritional supplement. 255. The method of embodiment 254, wherein the at least one nutritional supplement comprises an omega-3 fatty acid. 256. The method of embodiment 254, wherein the at least one nutritional supplement comprises a polyunsaturated fatty acid. 257. The method of embodiment 254, wherein the at least one nutritional supplement comprises a monounsaturated fatty acid. 258. The method of embodiment 221, wherein the population of cells is cultured using a non-serum media formulation. 259. The method of embodiment 221, wherein the population of cells is cultured using a mushroom-based media formulation. 260. A method of producing cultured meat for human consumption, the method comprising: obtaining a population of self-renewing cells, said cells capable of growing in suspension culture; culturing the population of self-renewing cells in suspension; inducing differentiation in the population of cells to form at least one of myocytes and adipocytes; and processing the population of cells into meat for human consumption. 261. The method of embodiment 260, wherein the meat is fish meat. 262. The method of embodiment 261, wherein the fish meat is sushi. 263. The method of embodiment 261, wherein the fish meat is surimi. 264. The method of embodiment 261, wherein the fish meat is suitable for raw consumption. 265. The method of embodiment 221, wherein the fish meat is cooked. 266. The method of embodiment 261, wherein the fish meat is salmon meat. 267. The method of embodiment 221, wherein the fish meat is sushi-grade salmon meat. 268. The method of embodiment 261, wherein the fish meat is tuna meat. 269. The method of embodiment 268, wherein the population of self-renewing cells is derived from a tuna selected from yellowfin, southern Bluefin, northern Bluefin, Thunnus alalunga, Thunnus atlanticus, and Thunnus obesus. 270. The method of embodiment 268, wherein the population of self-renewing cells is derived from Bluefin tuna. 271. The method of embodiment 221, wherein the fish meat is sushi-grade tuna meat. 272. The method of embodiment 261, wherein the inducing differentiation in the population of cells causes the population of cells to form myocytes and adipocytes. 273. The method of embodiment 261, wherein the fish meat is composed of at least 50% high glycolytic and anaerobic muscle fibers. 274. The method of embodiment 261, wherein the population of cells is derived from sea bass, tuna, mackerel, blue marlin, swordfish, yellowtail, salmon, or trout. 275. The method of embodiment 261, wherein the processing the population of cells into meat for human consumption comprises combining the population of cells with a second population of cells composed of myocytes or adipocytes. 276. The method of embodiment 261, wherein the population of cells is isolated as embryonic stem cells. 277. The method of embodiment 261, wherein the population of cells has been modified to induce pluripotency. 278. The method of embodiment 261, wherein the population of cells is isolated as multipotent adult stem cells. 279. The method of embodiment 261, wherein culturing comprises growing and expanding the population of cells in cell culture. 280. The method of embodiment 261, wherein inducing differentiation comprises exposing the population of cells to culture conditions that stimulate differentiation. 281. The method of embodiment 261, wherein inducing differentiation comprises exposing the population of cells to at least one growth factor that stimulates differentiation. 282. The method of embodiment 261, wherein culturing comprises growing the population of cells on a two dimensional surface. 283. The method of embodiment 261, wherein the population of cells forms non-textured tissue after differentiation. 284. The method of embodiment 261, wherein culturing comprises growing the population of cells in a media formulation comprising at least one nutritional supplement. 285. The method of embodiment 284, wherein the at least one nutritional supplement comprises an omega-3 fatty acid. 286. The method of embodiment 284, wherein the at least one nutritional supplement comprises a polyunsaturated fatty acid. 287. The method of embodiment 284, wherein the at least one nutritional supplement comprises a monounsaturated fatty acid. 288. The method of embodiment 261, wherein the population of cells is cultured using a non-serum media formulation. 289. The method of embodiment 261, wherein the population of cells is cultured using a mushroom-based media formulation. 290. A system for producing cultured tissues suitable for human consumption comprising: a reactor chamber comprising a plurality of micro-scaffolds that provide adhesion surfaces for cellular attachment; a population of self-renewing cells cultivated within bioreactor; a first source providing at least one maintenance media comprising components for maintaining the population of self-renewing cells without spontaneous differentiation; and a second source providing at least one differentiation media comprising components for differentiating the population of self-renewing cells into a specific lineage; wherein the reactor chamber receives maintenance media from the first source to cultivate the population of cells and receives differentiation media from the second source to differentiate the population of cells, wherein the population of cells generated in a single batch comprises cultured tissues suitable for human consumption and having a dry weight of at least 1 kg. 291. The system of embodiment 290, further comprising at least one sensor for monitoring the reactor chamber. 292. The system of embodiment 290, wherein the at least one sensor is a biosensor, a chemosensor, or an optical sensor. 293. The system of embodiment 290, wherein the at least one sensor is configured to monitor at least one of pH, temperature, oxygen, carbon dioxide, glucose, lactate, ammonia, hypoxanthine, amino acid(s), dopamine, and lipid(s). 294. The system of embodiment 290, further comprising at least one additional reactor chamber. 295. The system of embodiment 290, wherein the single batch has a dry weight of at least 5 kg. 296. The system of embodiment 290, further comprising a plurality of micro-scaffolds. 297. The system of embodiment 296, wherein the plurality of micro-scaffolds comprise glucomannan or alginate. 298. The system of embodiment 290, further comprising at least one 3D scaffold. 299. The system of embodiment 290, further comprising a third source providing at least one steatotic media comprising components for inducing steatosis or lipid accumulation in the population of cells. 300. The system of embodiment 299, wherein the components for inducing steatosis or lipid accumulation comprises linoleic acid, oleic acid, or a combination thereof 301. The system of embodiment 290, wherein the population of cells is cultured in media comprising at least one nutritional supplement. 302. The system of embodiment 301, wherein the at least one nutritional supplement comprises mushroom extract, soybean hydrolysate, or a combination thereof 303. A method for producing cultured fish tissue, the method comprising: culturing a population of fish pre-adipocytes and a population of fish satellite cells; inducing differentiation in the population of fish pre-adipocytes to form adipocytes; inducing differentiation in the population of fish satellite cells to produce myocytes; co-culturing the adipocytes and myocytes; and processing the adipocytes and myocytes into fish tissue for human consumption. 304. The method of embodiment 303, wherein the fish tissue comprises fast twitch muscle fibers. 305. The method of embodiment 303, wherein the fish tissue is salmon tissue. 306. The method of embodiment 303, wherein the fish tissue is tuna tissue. 307. The method of embodiment 303, wherein the fish tissue is trout tissue. 308. The method of embodiment 303, wherein the fish tissue is surimi. 309. The method of embodiment 303, wherein the fish tissue is sushi. 310. The method of embodiment 303, wherein the fish tissue is made for raw human consumption 311. The method of embodiment 303, wherein the fish tissue is cooked for human consumption. 312. The method of embodiment 303, wherein the adipocytes and myocytes are co-cultured in a media formulation comprising at least one nutritional supplement. 313. The method of embodiment 312, wherein the at least one nutritional supplement comprises an omega-3 fatty acid. 314. The method of embodiment 312, wherein the at least one nutritional supplement comprises a polyunsaturated fatty acid. 315. The method of embodiment 312, wherein the at least one nutritional supplement comprises a monounsaturated fatty acid. 316. The method of embodiment 261, wherein a non-serum media formulation is used for cell culturing. 317. The method of embodiment 261, wherein a mushroom-based media formulation is used for cell culturing. 318. A method for producing cultured fish tissue, the method comprising: culturing a population of fish pre-adipocytes and a population of fish satellite cells, said populations adapted for suspension culture; inducing differentiation in the population of fish pre-adipocytes to form adipocytes; inducing differentiation in the population of fish satellite cells to form myocytes; co-culturing the adipocytes and myocytes; and processing the adipocytes and myocytes into fish tissue for human consumption. 319. An edible composition comprising fish tissue produced from co-cultured myocytes and adipocytes. 320. An edible composition comprising fish tissue produced from pre-adipocytes and satellite cells. 321. A method for producing cultured fish meat for human consumption, the method comprising: obtaining a population of pre-adipocytes and a population of satellite cells; adapting the population of pre-adipocytes and the population of satellite cells to suspension culture; inducing differentiation in the population of pre-adipocytes and the population of satellite cells; co-culturing the populations in suspension culture; and processing the populations into fish meat for human consumption. 322. The method of embodiment 321, wherein the fish meat is sushi. 323. The method of embodiment 321, wherein the fish meat is surimi. 324. The method of embodiment 321, wherein the fish meat is suitable for raw consumption. 325. The method of embodiment 321, wherein the fish meat is cooked. 326. The method of embodiment 321, wherein the fish meat is salmon meat. 327. The method of embodiment 321, wherein the fish meat is sushi-grade salmon meat. 328. The method of embodiment 321, wherein the fish meat is tuna meat. 329. The method of embodiment 321, wherein the fish meat is sushi-grade tuna meat. 330. The method of embodiment 321, wherein the fish meat is trout meat. 331. The method of embodiment 321, wherein the fish meat is composed of at least 50% high glycolytic and anaerobic muscle fibers. 332. The method of embodiment 321, wherein the population of pre-adipocytes is derived from sea bass, tuna, mackerel, blue marlin, swordfish, yellowtail, salmon, or trout. 333. The method of embodiment 321, wherein the population of satellite cells is derived from sea bass, tuna, mackerel, blue marlin, swordfish, yellowtail, salmon, or trout. 334. The method of embodiment 321, wherein co-culturing comprises growing and expanding the populations in cell culture. 335. The method of embodiment 321, wherein inducing differentiation comprises exposing the population of pre-adipocytes to at least one growth factor that stimulates differentiation into adipocytes. 336. The method of embodiment 321, wherein inducing differentiation comprises exposing the population of satellite cells to at least one growth factor that stimulates differentiation into myocytes. 337. The method of embodiment 321, wherein culturing comprises growing the population of cells within a bioreactor. 338. The method of embodiment 321, wherein the myocytes and adipocytes form non-textured tissue after differentiation. 339. The method of embodiment 321, wherein the myocytes and adipocytes are cultured in a media formulation comprising at least one nutritional supplement. 340. The method of embodiment 339, wherein the at least one nutritional supplement comprises an omega-3 fatty acid. 341. The method of embodiment 339, wherein the at least one nutritional supplement comprises a polyunsaturated fatty acid. 342. The method of embodiment 339, wherein the at least one nutritional supplement comprises a monounsaturated fatty acid. 343. The method of embodiment 321, wherein a non-serum media formulation is used for cell culturing. 344. The method of embodiment 321, wherein a mushroom-based media formulation is used for cell culturing. 345. A fish product suitable for human consumption comprising fish meat produced from cultured myocytes and adipocytes. 346. A fish product suitable for human consumption comprising fish meat derived from cultured satellite cells and pre-adipocytes. 347. A fish product suitable for human consumption comprising fish meat produced from myocytes and adipocytes grown in suspension culture. 348. A method for producing cultured fish meat for human consumption, the method comprising: obtaining a population fish pre-adipocytes capable of growing in suspension culture; obtaining a population of fish satellite cells capable of growing in suspension culture; inducing differentiation in the population of fish pre-adipocytes and the population of fish satellite cells to form adipocytes and myocytes; co-culturing the adipocytes and myocytes in suspension culture comprising at least one nutritional supplement; and processing the population of cells into fish meat for human consumption. 349. The method of embodiment 348, wherein the fish meat is sushi. 350. The method of embodiment 348, wherein the fish meat is surimi. 351. The method of embodiment 348, wherein the fish meat is suitable for raw consumption. 352. The method of embodiment 348, wherein the fish meat is cooked. 353. The method of embodiment 348, wherein the fish meat is salmon meat. 354. The method of embodiment 348, wherein the fish meat is sushi-grade salmon meat. 355. The method of embodiment 348, wherein the fish meat is tuna meat. 356. The method of embodiment 348, wherein the fish meat is sushi-grade tuna meat. 357. The method of embodiment 348, wherein the fish meat is composed of at least 50% high glycolytic and anaerobic muscle fibers. 358. The method of embodiment 348, wherein the population of cells is derived from sea bass, tuna, mackerel, blue marlin, swordfish, yellowtail, salmon, or trout. 359. The method of embodiment 348, wherein inducing differentiation in (c) comprises exposing the population of pre-adipocytes and the population of satellite cells to culture conditions that stimulate differentiation. 360. The method of embodiment 348, wherein inducing differentiation in (c) comprises exposing the population of pre-adipocytes to at least one growth factor that stimulates differentiation. 361. The method of embodiment 348, wherein inducing differentiation in (c) comprises exposing the population of satellite cells to at least one growth factor that stimulate differentiation. 362. The method of embodiment 348, wherein the adipocytes and myocytes form non-textured tissue. 363. The method of embodiment 348, wherein the at least one nutritional supplement comprises an omega-3 fatty acid. 364. The method of embodiment 348, wherein the at least one nutritional supplement comprises a polyunsaturated fatty acid. 365. The method of embodiment 348, wherein the at least one nutritional supplement comprises a monounsaturated fatty acid. 366. The method of embodiment 348, wherein a non-serum media formulation is used for cell culturing. 367. The method of embodiment 348, wherein a mushroom-based media formulation is used for cell culturing. 368. A method of producing cultured liver tissue for human consumption, the method comprising: obtaining a population of cells; modifying the population of cells to generate a modified cell line configured to express at least one hepatocyte differentiation factor upon treatment with an induction agent; culturing the modified cell line; and treating the modified cell line with the induction agent to produce cultured liver tissue; and processing the cultured liver tissue for human consumption. 369. A method of producing steatotic liver tissue for human consumption, the method comprising: obtaining a hepatocyte cell line modified to express at least one steatotic factor upon treatment with an induction agent; culturing the hepatocyte cell line; and treating the hepatocyte cell line with an induction agent to produce steatotic liver tissue; and processing the steatotic liver tissue into a food product for human consumption. 370. A genetically modified cell line adapted for meat production, comprising: a first genetic construct comprising at least one pluripotency gene for promoting cell cycle progression; and a second genetic construct comprising at least one cell lineage gene for promoting differentiation, a regulatory factor configured to inactivate the at least one pluripotency gene, and an inducible promoter controlling expression of the at least one cell lineage gene and the regulatory factor. 371. A method of producing cultured tissue for human consumption, the method comprising: obtaining a population of self-renewing cells; culturing the population of self-renewing cells; inducing differentiation in the population of cells to form cultured tissue; and processing the cultured tissue for human consumption. 372. The method of embodiment 371, wherein obtaining the population of self-renewing cells comprises transitioning a population of cells from 2-dimensional adherent culture into 3-dimensional culture in a bioreactor. 373. The method of embodiment 371, wherein culturing comprises seeding the population of self-renewing cells on 3-dimensional micro-scaffolds. 374. The method of embodiment 3, wherein the 3-dimensional micro-scaffolds promote cell growth, adhesion, differentiation, or a combination thereof. 375. The method of embodiment 3, wherein the 3-dimensional micro-scaffolds are conjugated to at least one factor promoting cell growth, adhesion, differentiation, or a combination thereof. 376. The method of embodiment 375, wherein the micro-scaffolds comprise glucomannan, alginate, collagen, elastin, heparan sulfate, chondroitin sulfate, keratan sulfate, hyaluronic acid, laminin, fibronectin, or a combination thereof 377. The method of any one of embodiments 371-376, wherein the population of self-renewing cells comprises at least one cell that has been modified to undergo inducible differentiation. 378. The method of embodiment 377, wherein the at least one cell has been modified to incorporate: a first genetic construct comprising an open reading frame (ORF) of at least one pluripotency gene; and a second genetic construct comprising an open reading frame (ORF) of a regulatory factor configured to inactivate the at least one pluripotency gene. 379. The method of embodiment 378, wherein the population of self-renewing cells comprises at least one cell that undergoes at least 50 cell divisions during culturing. 380. The method of embodiment 378, wherein the regulatory factor is a recombinase, and the open reading frame (ORF) of at least one pluripotency gene is flanked by recombination sequences recognized by the recombinase such that expression of the recombinase catalyzes excision of the open reading frame (ORF) of at least one pluripotency gene. 381. The method of embodiment 378, wherein the at least one pluripotency gene comprises at least one of Hepatocyte Nuclear Factor 1 Alpha (HNF1, Forkhead Box A2 (FOXA2), and Hepatocyte Nuclear Factor 4 Alpha (HNF4). 382. The method of embodiment 378, wherein the at least one pluripotency gene comprises Myogenin (MyoG), Myogenic Differentiation 1 (MyoD), Myogenic Factor 6 (MRF4), Myogenic Factor 5 (MYF5), or any combination thereof. 383. The method of embodiment 378, wherein the second genetic construct further comprises: an open reading frame (ORF) of at least one differentiation gene; and an inducible promoter controlling expression of: the open reading frame (ORF) of the at least one differentiation gene; and the open reading frame (ORF) of the regulatory factor. 384. The method of embodiment 383, wherein inducing differentiation comprises exposing the at least one cell to an induction agent to induce expression of the ORF of at least one cell lineage gene and the ORF of the regulatory factor. 385. The method of embodiment 384, further comprising removing the induction agent after the population of self-renewing cells has been treated with the induction agent and before being the processing the cultured tissue for human consumption. 386. The method of embodiment 371, wherein inducing differentiation comprises generating myotubes within the population of self-renewing cells. 387. The method of embodiment 386, wherein inducing differentiation further comprises generating adipocytes within the population of self-renewing cells. 388. The method of any of embodiments 371-387, wherein the population of self-renewing cells comprises a first subset of cells that differentiates into myocytes and a second subset of cells that differentiates into adipocytes during the inducing differentiation in the population of cells to form cultured tissue. 389. The method of embodiment 371, wherein inducing differentiation comprises generating hepatocytes within the population of self-renewing cells. 390. The method of embodiment 389, wherein the population of self-renewing cells is derived from an avian species selected from duck, goose, chicken, and turkey. 391. The method of embodiment 389, further comprising inducing steatosis within at least one of the hepatocytes. 392. The method of embodiment 391, wherein the population of self-renewing cells comprises at least one cell modified to express at least one gene for enhancing steatosis upon treatment with an induction agent. 393. The method of embodiment 392, wherein the at least one cell is stably transformed using a construct comprising at least one open reading frame (ORF) encoding ATF4, ZFP423, LPIN1, PPAR, APOC3, APOE, ORL1, PEMT, MTTP, SREBP, STAT3, KLF6, or any combination thereof. 394. The method of any of embodiments 389-393, wherein inducing steatosis comprises incubating the hepatocytes in a culture medium comprising at least nutritional supplement. 395. The method of embodiment 394, wherein the at least one nutritional supplement comprises a polyunsaturated fatty acid, a monounsaturated fatty acid, or a combination thereof 396. The method of embodiment 394, wherein the at least one nutritional supplement comprises palmitic acid, oleic acid, docosahexaenoic acid, stearic acid, linoleic acid, linolenic acid, arachidonic acid, eicosapentaenoic acid, or a combination thereof. 397. The method of any one of embodiments 371-376, wherein the cultured tissue comprises octopus, squid, or cuttlefish muscle cells. 398. The method of any one of embodiments 371-376, wherein the cultured tissue comprises fish muscle tissue. 399. The method of embodiment 398, wherein the population of self-renewing cells is derived from sea bass, tuna, mackerel, blue marlin, swordfish, yellowtail, salmon, or trout. 400. The method of embodiment 398, wherein the fish muscle tissue is combined with separately cultured fish fat tissue during the processing the cultured tissue for human consumption. 401. The method of embodiment 371, wherein the population of cells is cultured using a non-serum media formulation. 402. The method of embodiment 401, wherein non-serum media formulation comprises a mushroom extract or soybean hydrolysate. 403. A cultured food product for human consumption, comprising the cultured tissue produced according to the methods of any one of embodiments 371-402. 404. The cultured food product of embodiment 403, wherein the cultured food product comprises packaging having a label indicating the cultured tissue was produced in a pathogen-free environment, a toxin-free environment, without force-feeding an animal, or any combination thereof 405. The cultured food product of embodiment 403, wherein the cultured tissue is processed into a plurality of slices and packaged to form the cultured food product. 406. A method of producing cultured tissue for human consumption, the method comprising: a) obtaining a population of self-renewing cells; b) culturing the population of self-renewing cells; c) inducing differentiation in the population of self-renewing cells to form cultured tissue; and d) processing the cultured tissue for human consumption. 407. The method of embodiment 406, wherein obtaining the population of self-renewing cells comprises transitioning a population of cells from 2-dimensional adherent culture into 3-dimensional culture in a bioreactor. 408. The method of embodiment 406, wherein the population of self-renewing cells comprises differentiated cells that have become immortalized. 409. The method of any one of embodiments 406-408, wherein inducing differentiation in the population of self-renewing cells comprises inducing transdifferentiation of cells in the population into myocytes, adipocytes, or a combination thereof. 410. The method of embodiment 406, wherein culturing comprises seeding the population of self-renewing cells on 3-dimensional micro-scaffolds. 411. The method of embodiment 410, wherein the 3-dimensional micro-scaffolds promote cell growth, adhesion, differentiation, or a combination thereof 412. The method of embodiment 410, wherein the 3-dimensional micro-scaffolds are conjugated to at least one factor promoting cell growth, adhesion, differentiation, or a combination thereof 413. The method of any one of embodiments 410-412, wherein the micro-scaffolds comprise at least one of hydrogel, chitosan, polyethylene terephthalate, collagen, elastin, heparan sulfate, chondroitin sulfate, keratan sulfate, hyaluronic acid, laminin, fibronectin, cellulose, hemicellulose, pectin, lignin, alginate, glucomannan, polycaprolactone (PCL), textured vegetable protein (TVP), textured soy protein (TSP), and acrylates. 414. The method of any one of embodiments 406-413, wherein the population of self-renewing cells comprises at least one cell that has been modified to undergo inducible differentiation. 415. The method of embodiment 414, wherein the at least one cell has been modified to incorporate: a) a first genetic construct comprising an open reading frame (ORF) of at least one pluripotency gene; and b) a second genetic construct comprising an open reading frame (ORF) of a regulatory factor configured to inactivate the at least one pluripotency gene. 416. The method of embodiment 415, wherein the population of self-renewing cells comprises at least one cell that undergoes at least 50 cell divisions during culturing. 417. The method of embodiment 415, wherein the regulatory factor is a recombinase, and the open reading frame (ORF) of at least one pluripotency gene is flanked by recombination sequences recognized by the recombinase such that expression of the recombinase catalyzes excision of the open reading frame (ORF) of at least one pluripotency gene. 418. The method of embodiment 415, wherein the second genetic construct comprises an ORF of at least one hepatocyte differentiation factor selected from Hepatocyte Nuclear Factor 1 Alpha (HNF1A), Forkhead Box A2 (FOXA2), and Hepatocyte Nuclear Factor 4 Alpha (HNF4A). 419. The method of embodiment 415, wherein the second genetic construct comprises at least one myogenic factor selected from Myogenin (MyoG), Myogenic Differentiation 1 (MyoD), Myogenic Factor 6 (MRF4), and Myogenic Factor 5 (MYF5). 420. The method of embodiment 415, wherein the second genetic construct comprises at least one adipogenic factor selected from Fatty Acid Binding Protein 4 (FABP4), Insulin-Responsive Glucose Transporter Type 4 (GLUT4), Adiponectin, C1Q And Collagen Domain Containing (ADIPOQ), 1-Acylglycerol-3-Phosphate O-Acyltransferase 2 (AGPAT2), Perilipin 1 (PLIN1), Leptin (LEP), and Lipoprotein Lipase (LPL). 421. The method of embodiment 415, wherein the second genetic construct further comprises: a) an open reading frame (ORF) of at least one differentiation gene; and b) an inducible promoter controlling expression of: i) the open reading frame (ORF) of the at least one differentiation gene; and ii) the open reading frame (ORF) of the regulatory factor. 422. The method of embodiment 421, wherein inducing differentiation comprises exposing the at least one cell to an induction agent to induce expression of the ORF of at least one cell lineage gene and the ORF of the regulatory factor. 423. The method of embodiment 421, further comprising removing the induction agent after the population of self-renewing cells has been treated with the induction agent and before being processed for human consumption in step d). 424. The method of embodiment 406, wherein inducing differentiation comprises generating myotubes within the population of self-renewing cells. 425. The method of embodiment 424, wherein inducing differentiation further comprises generating adipocytes within the population of self-renewing cells. 426. The method of any of embodiments 406-425, wherein the population of self-renewing cells comprises multipotent cells that are induced to differentiate into myocytes and adipocytes during step c). 427. The method of embodiment 426, wherein the multipotent cells comprise a first subpopulation of myosatellite cells and a second subpopulation of pre-adipocytes. 428. The method of embodiment 406, wherein inducing differentiation comprises generating hepatocytes within the population of self-renewing cells. 429. The method of embodiment 428, wherein the population of self-renewing cells is derived from an avian species selected from duck, goose, chicken, and turkey. 430. The method of embodiment 429, further comprising inducing steatosis within at least one of the hepatocytes. 431. The method of embodiment 430, wherein the population of self-renewing cells comprises at least one cell modified to express at least one gene for enhancing steatosis upon treatment with an induction agent. 432. The method of embodiment 431, wherein the at least one cell is stably transformed using a construct comprising an open reading frame (ORF) encoding ATF4, ZFP423, LPIN1, PPAR, APOC3, APOE, ORL1, PEMT, MTTP, SREBP, STAT3, or KLF6. 433. The method of any one of embodiments 430-432, wherein inducing steatosis comprises incubating the hepatocytes in a culture medium comprising at least nutritional supplement. 434. The method of embodiment 433, wherein the at least one nutritional supplement comprises a polyunsaturated fatty acid, a monounsaturated fatty acid, or a combination thereof. 435. The method of embodiment 433 or 434, wherein the at least one nutritional supplement comprises palmitic acid, oleic acid, docosahexaenoic acid, stearic acid, linoleic acid, linolenic acid, arachidonic acid, eicosapentaenoic acid, or a combination thereof 436. The method of any one of embodiments 406-427, wherein the cultured tissue comprises octopus, squid, or cuttlefish muscle cells. 437. The method of any one of embodiments 406-427, wherein the cultured tissue comprises fish muscle tissue. 438. The method of any one of embodiments 406-427, wherein the population of self-renewing cells is derived from sea bass, tuna, mackerel, blue marlin, swordfish, yellowtail, salmon, or trout. 439. The method of embodiment 437, wherein the fish muscle tissue is combined with separately cultured fish fat tissue during step d). 440. The method of any one of embodiments 406-439, wherein the population of cells is cultured using a non-serum media formulation. 441. The method of any one of embodiments 406-440, wherein non-serum media formulation comprises a mushroom extract or soybean hydrolysate. 442. A cultured food product for human consumption, comprising the cultured tissue produced according to the methods of any one of embodiments 406-441. 443. The cultured food product of claim 442, wherein the cultured food product comprises packaging having a label indicating the cultured tissue was produced in a pathogen-free environment, a toxin-free environment, without force-feeding an animal, or any combination thereof 444. The cultured food product of embodiment 442 or 443, wherein the cultured tissue is processed into a plurality of slices and packaged to form the cultured food product.

### EXAMPLES

The following illustrative examples are representative of embodiments of the systems, methods, and compositions described herein and are not meant to be limiting in any way.

### Example 1—Cultured Fish Meat Produced Using Embryonic Stem Cells

Embryonic stem cells are isolated from salmon embryos. The embryonic stem cells are first cultured using optimized media substrates and media formulations to achieve persistent cellular proliferation and maintenance of the de-differentiated state. The media formulations utilize synthetic serum-free media. The cells are cultured in a pathogen-free cell culture system. Next, the embryonic stem cells are induced to differentiate into myosatellite cells and pre-adipocytes. The myosatellite cells and pre-adipocytes are cultured and expanded to a desired quantity of cells. Next, the myosatellite cells and pre-adipocytes are differentiated into myocytes and adipocytes, which are then harvested and processed by centrifugation and compaction to generate the texture and consistency of fish meat.

### Example 2—Cultured Fish Meat Produced Using Induced Pluripotent Stem Cells

Fish fibroblasts are isolated from salmon. An episomal reprogramming strategy is employed to create induced pluripotent stem cells from the isolated fish fibroblasts without the use of classic viral reprogramming techniques. The induced pluripotent stem cells are cultured using optimized media substrates and media formulations to achieve persistent cellular proliferation and maintenance of the de-differentiated state. The media formulations utilize synthetic serum-free media. The cells are cultured in a pathogen-free cell culture system. Next, the iPS cells are expanded to a desired quantity of cells and then induced to differentiate into myocytes and adipocytes. Finally, the myocytes and adipocytes are harvested and processed by centrifugation and compaction to generate the texture and consistency of fish meat.

### Example 3—Cultured Fish Meat Produced Using Direct Cell Reprogramming

Differentiated fish fibroblasts are isolated from salmon. The fibroblasts are serially passaged until a cell line is selected that has capacity for continuous self-renewal (e.g., immortalized). The immortalized fibroblasts are grown in culture to a desired quantity, and then transdifferentiated. A reprogramming strategy using the overexpression of select genes is employed to directly reprogram the fibroblasts into myocytes and adipocytes without creating an intermediate pluripotent cell type. Accordingly, transdifferentiation allows for the immortalized fibroblasts to be converted into the desired cell type without requiring the use of stem cells.

### Example 4—Micro-Scaffolding System for Culturing Synthetic Foods

Cells are cultured using any of the techniques described in examples 1-6 using a bioreactor containing micro-scaffolds that allow for the attachment and growth of the adherent hepatocytes to generate small cellular structures capable of being grown in suspension. The micro-scaffolds are composed of a biocompatible material that biodegrades over time such that the cultured hepatocytes structures eventually no longer have any scaffolding material remaining. The hepatocyte structures are subsequently processed to produce foie gras.

### Example 5—3D Scaffolding System for Culturing Synthetic Foods

Cells are cultured using any of the techniques described in examples 1-6 using 3D scaffolds that guide the growth of the adherent hepatocytes to generate cellular structures approximating the size and shape of conventional avian livers. The 3D scaffolds are composed of a biocompatible material such as alginate that biodegrades over time such that the finished foie gras product no longer has any scaffolding material remaining. As a result, the hepatocytes are grown to approximate the conventional avian liver without requiring centrifugation and processing to generate foie gras having the desired texture and consistency.

### Example 6—Foie Gras Produced Using Conventional Techniques

Baby geese are raised and spend the first four weeks of their lives eating and growing. They are then transferred to cages and fed a high protein, high starch diet for another four weeks. At about eight to ten weeks, the birds are force fed by gavage, wherein two to four pounds of grain and fat are forced down the birds' throats using a feeding tube on a daily basis. The excess consumption of food causes the birds' livers to undergo steatosis in which the livers enlarge up to ten times or more than their normal size. During this process, the birds are exposed to various pathogens in the crowded and unsanitary conditions. Finally, the geese are slaughtered, and their livers harvested and sold as foie gras.

### Example 7—Sushi-Grade Salmon Produced Using Embryonic Stem Cells

Embryonic stem cells are isolated from salmon embryos. The embryonic stem cells are first cultured using optimized media substrates and media formulations to achieve persistent cellular proliferation and maintenance of the de-differentiated state. The media formulations utilize synthetic serum-free media. The cells are cultured in a pathogen-free cell culture system without exposure to toxins or heavy metals such as mercury that is often found in fish meat. Next, separate populations of embryonic stem cells are induced to differentiate into myocytes and adipocytes, respectively. In this case, the myocytes are differentiated to produce a desired ratio of around 80% fast twitch and around 20% slow twitch muscle fibers. The myocytes and adipocytes are cultured and expanded to a desired quantity of cells. Afterwards, the myocytes and adipocytes are harvested and processed by centrifugation and compaction to generate the texture and consistency of conventional sushi-grade salmon, or alternatively, surimi-style salmon.

### Example 8—Sushi-Grade Salmon

Pre-adipocytes and satellite cells are isolated from salmon fingerlings, and subsequently characterized and cultivated in cell culture as separate cell lines. Each cell line is cultured using optimized media formulations to adapt the cell lines to suspension culture. Afterwards, adipocyte and myocyte differentiation is induced in the pre-adipocyte and satellite cell lines, respectively. Next, the cell lines are co-cultured at an optimized ratio to produce a desired final ratio of myocytes to adipocytes. The media formulations utilize synthetic serum-free media comprising mushroom-derived extracts that replace fetal bovine serum. The cells are cultured in a pathogen-free cell culture system without exposure to toxins or heavy metals such as mercury that is often found in fish meat. The culture media is supplemented with high concentrations of free fatty acids such as oleic acid, thereby inducing the adipocytes to take in and storing an excess amount of extracellular fatty acids. The co-cultured myocytes and adipocytes are then harvested and processed by centrifugation and compaction to generate the texture and consistency of salmon surimi.

### Example 9—Isolation and Cultivation of Salmonid Stem Cells (Pre-Adipocyte, and Myosatellite)

Fish myocytes and adipocytes were targeted for development of fish-related foods based on their intrinsic regenerative capacity during early developmental stages. The cultivation of relevant salmon tissues ex vivo, including myocytes, adipocytes, hepatocytes, fibroblasts, and undifferentiated multipotent cell lineages is characterized and optimized. First, trout pre-adipocytes and myosatellite cells (capable of differentiating into myocytes) are isolated, cultured, and characterized. Trout myosatellite cells were isolated and then characterized as shown in FIGS. 5A-5D. Where present, insets magnify image details, and the scale bar is equal to 10 μm in all micrographs. Substantially pure populations of piscine myosatellite cells were successfully isolated and are shown in FIG. 5A with the myosatellite cells making up about 80% of the isolated cells. Next, these cells were characterized with relevant transcriptional markers. FIG. 5B shows RT-PCR results confirming the presence of hallmark genes (Mstn1a, Myf5) expressed in these isolated cells. Next, culture conditions were optimized for these cell lines. Culture media protocols were used to successfully differentiate myosatellite cells into mature myocytes (FIG. 5C). The sheets of myotubes differentiated from the myosatellite cells are shown in FIG. 5D. Pre-adipocytes were also differentiated into adipocytes.

In addition, salmon myosatellite cells (arrowheads) were co-cultured with salmon pre-adipocytes (arrows) for producing a food product comprising both muscle and fat cells or tissue as shown in FIG. 6A (scale bar is 100 μm). The pre-adipocytes were differentiated into adipocytes, and the myosatellite cells differentiated into myocytes (arrowhead) as shown in FIG. 6B (scale bar is 10 μm).

### Example 10—De Novo Creation of Induced Pluripotent Stem Cells Using Episomal Reprogramming

Pluripotent stem cells were targeted for development of cultured foods based on their lack of commitment to a cell lineage and having great versatility with respect to inducible differentiation. For example, myocytes, adipocytes, fibroblasts and other tissue components can be created from a single pool of pluripotent stem cells.

Induced pluripotent stem cells (iPSCs) are generated using episomal (non-integrating) reprogramming. The iPSCs form colonies growing on a monolayer of mouse embryonic fibroblast (MEF) feeder cells. These cells are then characterized for pluripotency markers, proliferative capacity, and efficiency of differentiation. Furthermore, culture conditions are optimized for the generated cell line with regards to cost, large scale synthesis, and maintenance of genomic/phenotypic stability.

### Example 11—Stem Cell Adaptation to Suspension Culture

Stem cell-based meat production represents an approach with multiple advantages over alternative cellular agriculture methodologies. First, the use of pluripotent stem cells is preferable to conventional myosatellite culture because the former possess indefinite replicative potential; this property obviates the need to repeatedly acquire muscle biopsies from animals during the process of production. Second, pluripotent stem cells are generally preferred over characterized immortal cell lines because they do not contain perturbations in cell cycle genetic networks or other related mutations. Finally, while the genetic and phenotypic characteristics of cell lines change in culture over time, the use of pluripotent stem cells with established differentiation protocols ensures the reproducibility of the final product. One challenge of stem cell-based meat production, however, is that stem cells generally are grown in 2-dimensional culture on a feeder cell line. Successfully adapting stem cells to 3-dimensional suspension culture for meat production would dramatically decrease the resource costs for cell culture.

Transitioning cells from 2-dimensional (e.g., cell culture dishes) to 3-dimensional suspension cultures is carried out and optimized to validate a method of scaling up cultured food production. Three dimensional suspension culture represents the first stage of scaling for food production because it offers the opportunity to grow significantly larger quantities of biological material with more efficient growth media utilization.

Growth of induced pluripotent stem cells (iPSCs) in suspension culture is facilitated by the creation of embryoid bodies (EBs), which are collections of stem cells that adhere to each other in lieu of an attachment surface on a plate. Like most mammalian cells, embryonic stem cells require signals from extracellular attachment points, and the process of acclimatizing them to the EB mode of growth often results in a high rate of cellular mortality and experimental variability. Protocols are refined to reliably acclimatize stem cells to growth in suspension culture, and to establish optimal culture conditions for growth and maintenance of pluripotency. One such method is the “hanging drop” technique, whereby cells are grown within a droplet of media, resulting in spontaneous formation of spheroids that are then acclimated to 3-dimensional culture conditions.

Cells are grown in media as “hanging drops” for 72 hours to form embryoid bodies. The embryoid bodies are then transferred to spinner flasks and grown in 3-dimensional suspension culture to allow scaling up of cell production.

Next, the proliferation kinetics under differing conditions of shear stress/laminar flow are assessed.

Finally, the induced pluripotent stem cells are differentiated in 3-dimensional cell culture.

### Example 12—Immortalized Cell Line Adaptation to Suspension Culture

An immortalized cell line derived from adult duck hepatocytes was generated by serial passaging the cultured hepatocytes and selecting colonies that proliferated at the highest rates. The immortalized cell line was then grown in media as “hanging drops” for 72 hours (FIG. 22A), after which the formation of spheroids became apparent. As shown in FIGS. 22A and 22B, the spheroids were then transferred to spinner flasks and grown in 3-dimensional suspension culture to allow scaling up of cell production.

Next, the proliferation kinetics under differing conditions of shear stress/laminar flow are assessed.

Finally, the immortalized duck hepatocytes are differentiated in 3-dimensional cell culture.

### Example 13—Cell Adaptation to Cell Suspension Culture and Differentiation Using Microscaffold Technology

In the case of multipotent stem cells (e.g., myosatellite cells or pre-adipocytes), the transition to 3-dimensional suspension culture was facilitated by the development of microscaffolds (also known as microcarriers). These molecules offer foci of attachment, promote survival in the context of 3-dimensional shear forces, and stimulate both growth and differentiation.

Novel microscaffolds were developed to overcome a fundamental challenge of 3-dimensional cell culture of ensuring that nutrients and other protective factors are accessible to cells deep within a growing tissue when these cells do not directly contact the cell culture medium. The microscaffolds provided an added benefit of enhancing cellular proliferative capacity by engaging integrins and other extracellular-sensing transmembrane proteins found in the extracellular environment. Finally, these microscaffolds are engineered to contribute gustatory and structural properties that determine the product's final taste and texture.

Glucomannan (a water-soluble polysaccharide derived from konjac) was identified after an initial screen for inexpensive, abundant, neutral-tasting, and partially-soluble polysaccharides. Glucomannan has a relatively neutral taste profile, while the concentration used could be used to influence the tensile elasticity of the final product. Next, microscaffolds were generated using glucomannan and tested in various formulations to promote differentiated duck and piscine cell growth. FIG. 23A shows fish myosatellite cells grown on glucomannan microscaffolds (10% w/v). These myosatellite cells were assessed for their ability to differentiate into myocytes on the microscaffolds. Five days after isolation and plating, myosatellite cells were able to differentiate into myocytes and form 3-dimensional myotubes more readily than with standard 2-dimensional conditions (e.g., plastic culture dishes without glucomannan microscaffolds). Both 2-dimensional and 3-dimensional cultures demonstrated improved cell proliferation, with enhanced 3-dimensional myotube formation observed in the case of myosatellite cells. FIG. 23B shows a negative control of myosatellite cells from the same preparation grown in identical cell culture conditions prior to differentiation.

Next, glucomannan-based gels used for meat production are characterized for thermostability and tensile elasticity. FIG. 24A shows duck fibroblasts (arrowheads) grown on glucomannan microscaffolds (arrows). FIG. 24B shows a representative glucomannan microscaffold. Thus, duck fibroblasts can successfully attach to and grow on glucomannan microscaffolds, demonstrating the potential to generate larger 3-D structures such as, for example, a liver (e.g. following differentiation of the fibroblasts into hepatocytes).

### Example 14—Cell Culture Media Optimization

Cell culture media (e.g., differentiated cell culture media) was optimized for reduced serum concentrations that continue to support cell viability and proliferative capacity. FIG. 17 shows the number of immortalized hepatocytes cultured in progressively decreasing concentrations of fetal bovine serum (FBS) in the presence of soybean hydrolysate (10 g/L). The number of hepatocytes and the percentage of FBS are graphed over time with the hepatocytes continuing to increase while serum concentrations gradually dropped from 10% to 0.8% by 20 days. The media supplementation of soybean hydrolysate allowed the serum requirements of the cultured cells to be reduced by 92%.

The cell culture media is improved and optimized through sourcing of animal-free alternatives to serum to reduce and/or eliminate animal-derived components from production. Duck fibroblasts were successfully grown in 10% shiitake mushroom extract after successive reduction of fetal bovine serum from the cell culture media (FIG. 18). Duck fibroblasts grown in serum-free Essential 8 media (FIG. 19A) were compared to control cultures grown in DMEM supplemented with 10% fetal bovine serum (FIG. 19B).

### Example 15—Salmonid Meat Product

The following components were used to create 1 gram of cell mass for a salmonid meat product prototype: 50 ml fetal bovine serum (FBS), 500 ml Dulbecco's Modified Eagle Media (DMEM), additional supplements such as pyruvate and non-essential amino acids, pipettes and culture dishes, and approximately 4 hours of labor. The successful transition to 3-dimensional culture reduced the labor time by half through elimination of the need for daily cell culture media changes and cell culture dishes, and reduction of pipette use, resulting in a relative decrease in resources needed to grow the cell mass. This corresponded to a roughly 50% reduction in price per pound. In addition, the transition to a plant-based media (e.g., soybean and/or cottonseed hydrolysate supplements) as described in example 17 further reduced the resource cost and price per pound by another 20%.

### Example 16—Microscaffold (Microcarrier) Optimization

Microscaffolds are generated using various naturally-occurring substrates such as agar, alginate, and long-chain neutral charge polysaccharides derivatives for more extensive testing with respect to promotion of cell growth, maintenance of phenotype, and the preservation of flavor and fundamental culinary properties. For example, glucomannan and other sugars can either be dissolved or polymerized by a chemical reaction (e.g., heating and cooling at a defined pH). Other microscaffolds can be generated by either dissolved in solution or polymerized and then broken into small pieces. Microscaffolds are generally irregularly shaped, and serve as points of attachment for small numbers of cells (as opposed to macroscaffolds, which are porous and enable cells to grow within them).

In addition to serving as components of cellular adhesion, certain extracellular matrix proteins (e.g., laminin, vitronectin, and others) also inhibit the differentiation of stem cells. Accordingly, microscaffold structures are developed that include recombinant extracellular matrix proteins. Such microscaffold hybrids (polysaccharide+matrix protein) can serve the dual purpose of promoting cell attachment and proliferation, while simultaneously inhibiting spontaneous and premature differentiation. Preliminary studies indicate that these engineered microscaffolds offer significant benefits in terms of cellular proliferation and maintenance of genotypic stability. These microscaffolds can be generated using materials that allow bioresorbtion during cell propagation, obviating the need to extract these materials prior to harvest of the resultant food products.

The microscaffolds are also engineered to incorporate protein growth factors, proteoglycans, and lipids for enhancement of microscaffold function. These hybrid particles/microscaffolds can modulate intracellular signaling toward enhanced proliferation and extended maintenance of the desired cellular phenotype. Various expression systems are tested for optimal yield and cost-effectiveness.

Accordingly, hybrid microscaffolds composed of polysaccharide/protein growth factor/extracellular matrix protein are generated and characterized. In addition, encapsulated lipids are incorporated within 3-dimensional cell culture. Some of these microscaffolds use neutral charge, long-chain polysaccharides as described above with extracellular matrix components, protein growth factors, and encapsulated fatty acids. Specific metrics for these engineering optimizations include cellular proliferative capacity and rate, genomic stability (particularly as it applies to telomerase expression/cellular senescence), efficiency of differentiation, and morphologic consistency during culture.

Finally, the microscaffolds are optimized to refine taste and texture. Similar to conventional meat, much of the structural/textural properties of the final product can result from the composition and physical chemistry of extracellular components. Fibrous and connective tissue, adhesive proteins, and the underlying architectural arrangement of all these components can collectively determine properties such as elasticity, fracture characteristics, thermostability, and taste. The engineered tissue generated using the methods described herein can incorporate one or more of these features and components to yield meats that are structurally indistinguishable from their conventional counterparts. Moreover, chemical profiling of conventional and ex vivo meat by mass spectrometry is carried out to further refine the composition and taste of its products. Additional development is carried out according to the following:

i. Characterization of Microscaffold Hybrids Consisting of Polysaccharides and Extracellular Matrix Proteins

Refine chemical synthesis methodology for polysaccharide/protein immobilization.

Characterize protein bioactivity and stability in 3-dimensional culture.

ii. Establish Functional Cellular Assays Pertaining to Microscaffold Growth, Namely: Proliferative Capacity, Proliferative Rate, Morphologic Metrics, and Efficiency of Differentiation

Synthesize polysaccharide/protein growth factor/extracellular matrix protein hybrid microscaffolds.

Efficiently immobilize recombinant protein growth factors onto described neutral-charge polysaccharide backbones.

Quantify cellular responses to growth on these hybrid microscaffolds, including analysis of relevant intracellular signaling pathways, proliferative capacity and rate, cell morphology, and genetic profile (transcriptome analysis).

iii. Incorporate Encapsulated Lipids within 3-Dimensional Cell Culture

Efficient production of encapsulated lipid microparticles (lipid species can include: unsaturated, polyunsaturated, saturated, omega-3 fatty acids, etc.).

Incorporation into the described polysaccharide/protein hybrid microscaffolds, with quantification of resultant cellular response (functional assays including relevant intracellular signaling, proliferative capacity and rate, cell morphology, and transcriptome analysis).

iv. Refine Taste/Texture

Refine final product preparation to develop a minimal viable product for piscine cell lines.

Optimize protocols for controlling lipid oxidation for piscine adipocytes.

Develop controlled taste tests to isolate the impact of each upstream product development decision on taste, texture, and appearance.

Optimize growth media formulation, scaffold choice, and lipid additions for each final product.

### Example 17—Characterization and Optimization of Low-Cost, Animal Component-Free, Culture Media

Fetal bovine serum (FBS) is the largest initial contributor to production costs in cellular agriculture. However, FBS is poorly-defined with respect to its components, inconsistent from lot to lot, and relies upon animal sources for harvest. Accordingly, described herein are novel species- and cell-specific media formulations that are 1) free of animal serum, 2) completely defined, 3) cost-effective for large scale production, and 4) appropriate for food production.

### Media for Stem Cells

Serum-free formulations that promote stem cell proliferation and inhibit differentiation of stem cells are generated. Such defined media formulations can be optimized for fish stem cells and use animal-free components such as plant-derived supplements. These formulations are optimized with respect to various growth factors to both promote stem cell growth and inhibit spontaneous differentiation in culture.

One approach is the in-house reconstitution of defined media formulations. An example of this approach is the published formulation of Essential 8™ medium, for which the eight base components (including recombinant proteins) may be separately sourced to support large-scale stem cell growth. Of these eight components, the price of four (recombinant transferrin, TGF-beta, insulin, and FGF2) by far outweigh the others (basal media, ascorbic acid, selenium and sodium bicarbonate) because of the costs associated with recombinant protein expression and purification. An additional requisite component in existing media for stem cell cultures is an extracellular matrix protein (such as laminin) that must similarly be expressed and purified, adding to the cost of these culture systems.

Various approaches to the production of cultured meat products can include recombinant expression of components, with in-house media reconstitution (detailed below) and development of a conditioned media system (detailed below).

### Recombinant Protein Expression

Recombinant proteins are expressed, purified, and then incorporated into media formulations which are assessed for ability to support and promote cell growth and proliferation. Expression systems include algae, bacteria, yeast, insect, and mammalian cell cultures. Although the expression and purification of individual proteins carries an initially high cost, this process permits a higher level of precision for the titration of protein concentrations in cell culture. Such precision aids in the process of defining requisite cell culture components. A peptide and protein screen is developed to aid in the optimization of cell culture components in each established cell line.

### Conditioned Media

The ability of cell lines to secrete growth factors that promote the viability and proliferation of other cells is the central principle underlying conditioned media or co-culture systems. Certain cell lines are evaluated for their ability to condition media with particular growth factors necessary for large-scale production of meat. In particular, cell lines that overexpress factors such as hepatocyte growth factor (HGF), fibroblast growth factor-2 (FGF2), and leukemia inhibitory factor (LIF) can improve cost-efficiency over expression and purification of the recombinant forms of these proteins.

Various development paths are pursued in parallel towards the creation of a cultured meat product. In the case of minced salmon, precursor cells (myosatellite cells and pre-adipocytes) are terminally differentiated into myocytes and adipocytes both together and separately, in order to establish an optimal method for salmon meat production. In addition, studies are carried out assessing the role of fibroblasts in promoting stable cell culture and in defining the textural qualities of the finished product.

### Characterization and Optimization of Low-Cost, Animal Component-Free, Culture Media Formulations

The various approaches can be refined and optimized to increase production efficiency and decrease costs.

Plant-based media optimization. FBS is reduced or eliminated from cell culture growth media by gradually transitioning cell lines to plant-based culture media supplemented with a plant-derived extract such as soybean hydrolysate or mushroom extract (see FIGS. 17-19). In addition, studies are carried out identifying lower cost, plant-based alternatives for supplements such as pyruvate and non-essential amino acids either through custom formulations or off-the-shelf products available through industrial-scale suppliers.

Proprietary low-cost media formulation. Further refine the plant-based media formulation, reducing pre-mixed DMEM requirements by moving to lower cost formulations.

Proprietary low-cost media is further optimized. Refine the plant-based media formulation by moving from higher-cost sources of animal-free recombinant proteins (e.g., albumin, transferrin, insulin) to industrial quantities and pricing for plant-derived protein components. In addition, continue improving the workflow and process automation, reducing labor requirements.

Final optimization to media components. Move from small scale spinning flasks and mechanical rocker systems to industrial grade bioreactors permitting reuse of media, larger scale tissue culture, and significant reductions in labor costs.

Approaches to the development of low-cost stem-cell growth media include:

Create conditioned media system for the expression of growth factors that support both stem cell and differentiated cell proliferation.

Optimize recombinant protein expression for cell culture components.

Conduct peptide/protein screen to improve cell proliferation rate, maintain de-differentiated state of stem cells, and increase efficiency of terminal differentiation.

Continue to improve workflow and process automation to reduce the labor required.

### Example 18—Transition to Medium-Scale Production Using Bioreactors

Bioreactors are adapted for the efficient growth of 3-dimensional cultures. This process is achieved by satisfying 3 sub-objectives: i) refining small-scale bioreactors for efficient tissue growth, ii) procuring and adapting medium-scale bioreactors to supply a minimum viable product to two restaurants, and iii) developing large-scale bioreactors under an economic model for large-scale meat production.

i. Refine Current Small-Scale Bioreactors

Efficient bioreactor design can minimize waste in culture media (with the recycling of components such as buffers) and provide the seamless transition from one culture media to another (e.g. stem cell growth media versus myocyte differentiation media). Preliminary studies entail optimizing laminar and shear flow in small-scale 3-dimensional cultures to optimize long-term cellular spheroid growth. These initial studies assess growth rates among different differentiated piscine cell lines (with or without microscaffolds).

ii. Procure and Adapt Medium-Scale Bioreactors

Approximately 100 liters of bioreactor volume is used to produce about two pounds of cell mass per week. This volume is supplied through five 20 L rocker bioreactors, which can supply the desired cell mass at a steady quantity per week based on a 4-6 week cell growth time. Alternatively, this volume is supplied through twenty 5 L bioreactors. In some cases, the bioreactors (or other containers used for cell culturing, growth, and/or differentiation) have a volume no larger than 5 liters.

Commercially-available bioreactors are configured for pharmaceutical applications and require the use of costly disposable bags for each tissue harvest. These bioreactors are down-scaled to remove unnecessary features to enable increased longer-term resource efficiency in cultured tissue production.

iii. Develop Model for Large-Scale Meat Production

Large-scale bioreactors (e.g., disposable bag and stainless steel designs) are assessed for suitability for large scale ex vivo meat production.

The transition to medium-scale production using bioreactors can be accomplished according to the methods described below.

a. Refine Current Small-Scale Bioreactors

Evaluate optimal media change frequency.

Assess opportunity for media reuse/recycling.

Identify optimal density at the time of harvest.

Identify methods for tracking cell growth (spheroids versus individual cells).

b. Procure and Adapt Medium-Scale Bioreactors

Determine optimal media volume per kilogram produced (validate 1:50 ratio assumption).

Optimize media changing regimen for each cell line.

Evaluate optimal fluid dynamics (shear stress, impeller type, speed) for various cell cultures and temperatures.

Integrate a conditioned media model of cell growth within the bioreactor system.

### Example 19—Cultured Food Product Optimization and Refinement

Further work is performed to refine product texture, taste, and nutritional composition. Increases to production efficiency are obtained by continued optimization of growth media formulations, engineering media recycling fluidics, minimizing sterile production waste (e.g., disposable plastics), and incorporating automation in the production pipeline.

### Example 20—Cultured Foie Gras Produced Using Embryonic Stem Cells

Embryonic stem cells are isolated from Eyal-Giladi and Kochav Stage 10 (EGK-X) avian embryos. The embryonic stem cells are first cultured using optimized media substrates and media formulations to achieve persistent cellular proliferation and maintenance of the de-differentiated state. The media formulations utilize synthetic serum-free media. The cells are cultured in a pathogen-free cell culture system. Next, the embryonic stem cells are induced to differentiate into hepatocytes. The hepatocytes are cultured and expanded to a desired quantity of cells. The culture media is supplemented with high concentrations of free fatty acids such as oleic acid, thereby inducing the hepatocytes to undergo steatosis by taking in and storing an excess amount of extracellular fatty acids. The steatotic hepatocytes are then harvested and processed by centrifugation and compaction to generate the texture and consistency of conventional foie gras. This foie gras product lacks texture of skeletal muscle meats. Moreover, the foie gras is substantially composed of only hepatocytes unlike skeletal muscle meats that include myocytes, endothelial cells, and adipose cells. The finished foie gras is firm, light-colored, and lacks any of the veins or blemishes that are often found in conventional foie gras. Accordingly, the foie gras is packaged for sale with a label indicating grade A quality and that it was made without force feeding.

### Example 21—Cultured Foie Gras Produced Using Induced Pluripotent Stem Cells

Avian dermal fibroblasts are isolated from a goose. An episomal reprogramming strategy is employed to create induced pluripotent stem cells from the isolated dermal fibroblasts without the use of classic viral reprogramming techniques. The induced pluripotent stem cells are cultured using optimized media substrates and media formulations to achieve persistent cellular proliferation and maintenance of the de-differentiated state. The media formulations utilize synthetic serum-free media. The cells are cultured in a pathogen-free cell culture system. Next, the iPS cells are induced to differentiate into hepatocytes and expanded to a desired quantity of cells. The culture media is supplemented with high concentrations of free fatty acids such as oleic acid, thereby inducing the hepatocytes to undergo steatosis by taking in and storing an excess amount of extracellular fatty acids. The steatotic hepatocytes are then harvested and processed by centrifugation and compaction to generate the texture and consistency of conventional foie gras.

### Example 22—Cultured Foie Gras Produced Using Direct Cell Reprogramming

Avian dermal fibroblasts are isolated from a duck. A reprogramming strategy using the overexpression of transcription factors is employed to directly reprogram the dermal fibroblasts into hepatocytes without creating an intermediate pluripotent cell type. The hepatocytes are expanded to a desired quantity of cells and grown in culture media supplemented with high concentrations of free fatty acids such as oleic acid, which cause the hepatocytes to undergo steatosis by taking in and storing an excess amount of extracellular fatty acids. The steatotic hepatocytes are then harvested and processed by centrifugation and compaction to generate the texture and consistency of conventional foie gras.

### Example 23—Cultured Foie Gras Produced Using Immortalized Mature Hepatocytes

Mature avian hepatocytes are isolated from duck liver. The hepatocytes are immortalized using classical techniques such as transformation with SV40 Large T Antigen or spontaneous hepatocyte immortalization by sequentially passaging the hepatocytes until spontaneous mutations arise that result in immortalization. The immortalized hepatocytes are expanded to a desired quantity of cells and grown in culture media supplemented with high concentrations of free fatty acids such as oleic acid, which cause the hepatocytes to undergo steatosis by taking in and storing an excess amount of extracellular fatty acids. The steatotic hepatocytes are then harvested and processed by centrifugation and compaction to generate the texture and consistency of conventional foie gras.

### Example 24—Cultured Foie Gras Produced Using Nascent Hepatic Stem Cells

Hepatic stem or progenitor cells are isolated from duck liver. The hepatic stem cells are cultured using optimized media substrates and media formulations to achieve persistent cellular proliferation and maintenance of the pluripotent state. The media formulations utilize synthetic serum-free media. The cells are cultured in a pathogen-free cell culture system. Next, the hepatic stem cells are induced to differentiate into mature hepatocytes and expanded to a desired quantity of cells. The culture media is supplemented with high concentrations of free fatty acids such as oleic acid, thereby inducing the hepatocytes to undergo steatosis by taking in and storing an excess amount of extracellular fatty acids. The steatotic hepatocytes are then harvested and processed by centrifugation and compaction to generate the texture and consistency of conventional foie gras.

### Example 25—Genetic Modulation of Cultured Hepatocytes to Induce Steatosis for Producing Foie Gras

Hepatocytes are obtained using any of the procedures described in examples 1-5, except the step of culturing the hepatocytes in a lipid enriched culture medium has been replaced with a step that genetically manipulates the metabolic pathway(s) responsible for lipid metabolism. In this case, the genetic manipulation results in the accumulation of lipid droplets within the cytoplasm of the hepatocytes, thereby resulting in steatosis.

The core technologies described herein is applied to the production of various meats such as trout, tuna, other seafood products, and avian meats based on similar goals of environmental, ethical, and nutritional benefits.

What is claimed is:

1. A method of producing cultured tissue for human consumption, the method comprising:
a) obtaining a population of self-renewing cells;
b) culturing the population of self-renewing cells;
c) inducing differentiation in the population of self-renewing cells to form cultured tissue; and
d) processing the cultured tissue for human consumption.

2. The method of claim 1, wherein obtaining the population of self-renewing cells comprises transitioning a population of cells from 2-dimensional adherent culture into 3-dimensional culture in a bioreactor.

3. The method of claim 1, wherein the population of self-renewing cells comprises differentiated cells that have become immortalized.

4. The method of any one of claims 1-3, wherein inducing differentiation in the population of self-renewing cells comprises inducing transdifferentiation of cells in the population into myocytes, adipocytes, or a combination thereof.

5. The method of claim 1, wherein culturing comprises seeding the population of self-renewing cells on 3-dimensional micro-scaffolds.

6. The method of claim 3, wherein the 3-dimensional micro-scaffolds promote cell growth, adhesion, differentiation, or a combination thereof.

7. The method of claim 3, wherein the 3-dimensional micro-scaffolds are conjugated to at least one factor promoting cell growth, adhesion, differentiation, or a combination thereof.

8. The method of claim 7, wherein the micro-scaffolds comprise at least one of hydrogel, chitosan, polyethylene terephthalate, collagen, elastin, heparan sulfate, chondroitin sulfate, keratan sulfate, hyaluronic acid, laminin, fibronectin, cellulose, hemicellulose, pectin, lignin, alginate, glucomannan, polycaprolactone (PCL), textured vegetable protein (TVP), textured soy protein (TSP), and acrylates.

9. The method of claim 8, wherein the population of self-renewing cells comprises at least one cell that has been modified to undergo inducible differentiation.

10. The method of claim 9, wherein the at least one cell has been modified to incorporate:
a) a first genetic construct comprising an open reading frame (ORF) of at least one pluripotency gene; and
b) a second genetic construct comprising an open reading frame (ORF) of a regulatory factor configured to inactivate the at least one pluripotency gene.

11. The method of claim 10, wherein the population of self-renewing cells comprises at least one cell that undergoes at least 50 cell divisions during culturing.

12. The method of claim 10, wherein the regulatory factor is a recombinase, and the open reading frame (ORF) of at least one pluripotency gene is flanked by recombination sequences recognized by the recombinase such that expression of the recombinase catalyzes excision of the open reading frame (ORF) of at least one pluripotency gene.

13. The method of claim 10, wherein the second genetic construct comprises an ORF of at least one hepatocyte differentiation factor selected from Hepatocyte Nuclear Factor 1 Alpha (HNF1A), Forkhead Box A2 (FOXA2), and Hepatocyte Nuclear Factor 4 Alpha (HNF4A).

14. The method of claim 10, wherein the second genetic construct comprises at least one myogenic factor selected from Myogenin (MyoG), Myogenic Differentiation 1 (MyoD), Myogenic Factor 6 (MRF4), and Myogenic Factor 5 (MYF5).

15. The method of claim 10, wherein the second genetic construct comprises at least one adipogenic factor selected from Fatty Acid Binding Protein 4 (FABP4), Insulin-Responsive Glucose Transporter Type 4 (GLUT4), Adiponectin, C1Q And Collagen Domain Containing (ADIPOQ), 1-Acylglycerol-3-Phosphate O-Acyltransferase 2 (AGPAT2), Perilipin 1 (PLIN1), Leptin (LEP), and Lipoprotein Lipase (LPL).

16. The method of claim 10, wherein the second genetic construct further comprises:
a) an open reading frame (ORF) of at least one differentiation gene; and
b) an inducible promoter controlling expression of:
i. the open reading frame (ORF) of the at least one differentiation gene; and
ii. the open reading frame (ORF) of the regulatory factor.

17. The method of claim 16, wherein inducing differentiation comprises exposing the at least one cell to an induction agent to induce expression of the ORF of at least one cell lineage gene and the ORF of the regulatory factor.

18. The method of claim 17, further comprising removing the induction agent after the population of self-renewing cells has been treated with the induction agent and before being processed for human consumption in step d).

19. The method of claim 1, wherein inducing differentiation comprises generating myotubes within the population of self-renewing cells.

20. The method of claim 19, wherein inducing differentiation further comprises generating adipocytes within the population of self-renewing cells.

21. The method of any of claims 1-20, wherein the population of self-renewing cells comprises multipotent cells that are induced to differentiate into myocytes and adipocytes during step c).

22. The method of claim 20, wherein the multipotent cells comprise a first subpopulation of myosatellite cells and a second subpopulation of pre-adipocytes.

23. The method of claim 1, wherein inducing differentiation comprises generating hepatocytes within the population of self-renewing cells.

24. The method of claim 23, wherein the population of self-renewing cells is derived from an avian species selected from duck, goose, chicken, and turkey.

25. The method of claim 23, further comprising inducing steatosis within at least one of the hepatocytes.

26. The method of claim 25, wherein the population of self-renewing cells comprises at least one cell modified to express at least one gene for enhancing steatosis upon treatment with an induction agent.

27. The method of claim 26, wherein the at least one cell is stably transformed using a construct comprising an open reading frame (ORF) encoding ATF4, ZFP423, LPIN1, PPAR, APOC3, APOE, ORL1, PEMT, MTTP, SREBP, STAT3, or KLF6.

28. The method of claim 27, wherein inducing steatosis comprises incubating the hepatocytes in a culture medium comprising at least nutritional supplement.

29. The method of claim 28, wherein the at least one nutritional supplement comprises a polyunsaturated fatty acid, a monounsaturated fatty acid, or a combination thereof.

30. The method of claim 28, wherein the at least one nutritional supplement comprises palmitic acid, oleic acid, docosahexaenoic acid, stearic acid, linoleic acid, linolenic acid, arachidonic acid, eicosapentaenoic acid, or a combination thereof.

31. The method of claim 16, wherein the cultured tissue comprises octopus, squid, or cuttlefish muscle cells.

32. The method of claim 16, wherein the cultured tissue comprises fish muscle tissue.

33. The method of claim 32, wherein the population of self-renewing cells is derived from sea bass, tuna, mackerel, blue marlin, swordfish, yellowtail, salmon, or trout.

34. The method of claim 32, wherein the fish muscle tissue is combined with separately cultured fish fat tissue during step d).

35. The method of claim 1, wherein the population of cells is cultured using a non-serum media formulation.

36. The method of claim 35, wherein non-serum media formulation comprises a mushroom extract or soybean hydrolysate.

37. A cultured food product for human consumption, comprising the cultured tissue produced according to the methods of any one of claims 1-36.

38. The cultured food product of claim 37, wherein the cultured food product comprises packaging having a label indicating the cultured tissue was produced in a pathogen-free environment, a toxin-free environment, without force-feeding an animal, or any combination thereof.

39. The cultured food product of claim 37, wherein the cultured tissue is processed into a plurality of slices and packaged to form the cultured food product.
